(19) United States
(12) Patent Application Publication
(10) Pub. No.: US 2007/0092942 A1
(43) Pub. Date: Apr. 26, 2007

(54) Ubiquitin specific enzymes of family herpesviridae

(76) Inventors: Gregory Korbel, New Haven, CT (US); Hidde L. Ploegh, Brookline, MA (US); Lisa M. Kattenhorn, Cambridge, MA (US); Christian Schlieker, Cambridge, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/506,450

(22) Filed: Aug. 18, 2006

Related U.S. Application Data
(60) Provisional application No. 60/709,360, filed on Aug. 18, 2005.

(51) Int. Cl. C07H 21/04; C12P 21/06; C12N 9/64

(52) U.S. Cl. 435/69.1; 435/226; 435/320.1; 435/325; 536/23.2

(57) ABSTRACT

Disclosed is a new class of deubiquitinating enzymes that are conserved across the family Herpesviridae.

### RELATED APPLICATIONS

This application claims priority to U.S. Ser. No. 60/709,360, filed Aug. 18, 2005. The contents of this application are incorporated by reference in their entirety.

### STATEMENT AS TO FEDERALLY SPONSORED RESEARCH

This invention was made with U.S. government support under United States NIH Grant No. AI057182. The United States Government has certain rights in the invention.

### TECHNICAL FIELD

This invention relates to post-translational modification of proteins.

### BACKGROUND OF THE INVENTION

Post-translational modification of proteins by ubiquitin (Ub) plays a central role in protein turnover, but the importance of this modification extends to processes as diverse as membrane trafficking and transcription. Ub can be appended post-translationally to the N-terminus or, more commonly, the lysine side chain of target proteins. Ubiquitination in mammalian cells is achieved through a cysteine-based enzymatic cascade comprised of a single ATP-dependent Ub-activating enzyme (E1), several dozen Ub-carrier proteins (E2s) that acquire Ub in thiolester linkage from the E1-Ub intermediate, and hundreds of Ub ligases (E3s) that participate in the transfer of Ub to substrate proteins. A subset of the E3s attach Ub to pre-existing ubiquitinated proteins in a process of chain extension polymerization, resulting in polyubiquitinated substrates.

The modification of proteins with Ub is reversed by deubiquitinating enzymes (DUBs), nearly 100 of which are predicted in the human genome. These enzymes catalyze the hydrolysis of the peptide and isopeptide bonds that link the C-terminal glycine of Ub to the amino groups of substrate proteins. Most of the known DUBs are cysteine proteases, characterized by a Cys-His-Asp catalytic triad. They have been further classified as either Ub C-terminal hydrolases (UCHs) or as ubiquitin specific proteases (USPs). UCHs are known to hydrolyze Ub electrophiles with small C-terminal leaving groups, but can also catalyze the removal of Ub from isopeptide-linked peptides of at least 13 residues in length. Members of the USP family are characterized by conserved Cys- and His-box motifs, which are flanked by N- and C-terminal extensions that differ in length and sequence between family members. These extensions are believed to modulate interactions with other proteins, including putative substrates. Members of the ovarian tumor (OTU)/Cezanne families, each distinct from the UCHs and USPs, are more recent additions to the list of DUBs. Finally, Rpn11/POH1, a metalloprotease whose activity is required for the orderly degradation of Ub-conjugated proteins, has been identified in association with the proteasome and is included within the DUB family.

### SUMMARY

The invention features a new class of deubiqutinating enzymes that are conserved across the family Herpesviridae. These enzymes bear little or no homology to cellular deubiqutinating enzymes and are therefore useful to selectively inhibit viral activity and/or replication.

In one aspect, the invention provides a Herpes virus ubiquitin specific protease containing a Cys box, the Cys box being selected from the group consisting of SEQ ID NO:1 or SEQ ID NO:2 and a His box being selected from SEQ ID NO:3 and SEQ ID NO:4. The protease is encoded by a naturally-occurring Herpes virus gene sequence. Preferably, the protease is not encoded by a naturally-occurring eukaryotic cell, e.g., a mammalian cell, gene sequence. The location of the Cys box is N-terminal to the location of the His box, and the Cys box and His box are separated by 75 to 90 amino acids. For example, the Cys box and His box are separated by 75-85 amino acids. In specific Herpes enzymes, the boxes are separated by 78, 79, 80, 81, 82, 83, 84, or 85 amino acids. Exemplary Cys box sequences include SEQ ID NO:5, 6, 7, 8, 9, 10, 11, and 12 (see FIG. 3B), and exemplary His box sequences include SEQ ID NO: 13, 14, 15, 16, 17, 18, 19, and 20 (see FIG. 3B).

Also within the invention is an inhibitor of a deubiquitinating enzyme that preferentially inhibits a Herpes virus enzyme compared to a cellular (e.g., mammalian) enzyme. For example, in one embodiment of the invention, the inhibitor is a cysteine protease inhibitor. The inhibitor preferentially or specifically binds to a Herpes virus DUB compared to other, e.g., cellular, DUBs. The inhibitor binds to a DUB encoded by a Herpes virus at least 10% 25%, 50%, 2-fold, 5-fold, 10-fold or more compared to the level of binding of the compound to a cellular DUB. Preferably, the inhibitor is a small molecule rather than a peptide. In one embodiment, the inhibitor is a methyl ketone compound such as an alpha-halogenated methyl ketone compound. For example, the halogen is chlorine or fluorine, bromine, iodine, or astatine. Exemplary compounds include Z-Z-VA-L-A-cmk, Z-VA-L-A-fmk, Z-VAG-cmk, and Z-AA-L-A-fmk. In another embodiment, the inhibitor is an inhibitor that inhibits expression of the DUB by an RNA interference mechanism. For example, the inhibitor can be a short interfering RNA (siRNA) comprising a strand that corresponds to the sequence of the gene that encodes the DUB, e.g., is at least partly complementary to the mRNA that encodes the DUB. See, e.g., U.S. Pat. Nos. 7,078,196 and 7,056,704 for nonlimiting descriptions of siRNA.

Given the high conservation among members of the family Herpesviridae and the lack of homology to cellular deubiqutinating enzymes, the invention also provides a method of selectively inhibiting Herpes virus replication by contacting a virally-infected cell with the inhibitor(s) described above as well as a method of inhibiting a Herpes virus infection in a subject by administering the same inhibitor(s) to the subject. These methods are useful to treat human clinical disease associated with infection by this class of virus as well as to treat numerous veterinary pathologies in pets such as dogs, cats, and the like as well as livestock such as cows, horses, pigs, poultry and the like. For example, equine and bovine Herpes viruses are known to cause wasting and abortions in pregnant animals; such infections are inhibited and symptoms of the infection reduced by inhibiting the activity of this class of deubiquinating enzymes. Also, poultry are subject to Marek's Disease, which is caused by an infection with Marek's Disease Virus (MDV), a member of Herpesviridae.

The inhibitors and methods described herein are useful to selectively inhibit enzyme activity and MDV virus replication in poultry as well as other animals such as rodents, other birds (e.g., turkeys, parrots), and monkeys. In certain embodiments of the invention the inhibitor is administered to a human subject infected by a herpesvirus selected from HHV-1, HHV-2, HHV-3, HHV-4, HHV-5, HHV-6, HHV-7, and HHV-8. In certain embodiments of the invention the inhibitor is administered to a subject suffering from or at risk of a disease associated with infection by a human herpesvirus. Examples of human diseases associated with herpesvirus infection include chicken pox, shingles, rosacea, and a variety of tumors such as nasopharyngeal cancer, Kaposi's sarcoma, primary effusion lymphoma, multicentric Castleman's disease. Other conditions include oral herpes and genital herpes. Inhibiting the activity of an HSV DUB identified herein is useful treating these and other conditions. The inhibitors could be administered either prophylatically, i.e., prior to the onset of symptoms (e.g., in an individual exposed or suspected of being exposed to a herpesvirus), or after the onset of symptoms. In one embodiment the individual is immunocompromised. For example, the individual may suffer from HIV infection.

### BRIEF DESCRIPTION OF THE DRAWINGS

FIGS. 1A-C are a series of photographs showing a new DUB is labeled in HSV-1 infected cell lysates. (A) HFFs, either uninfected or infected with HSV-1, were harvested at the indicated times post infection, lysed and incubated with HAUbVME. Proteins were separated by SDS-PAGE and immunoblotted with anti-HA antibody. (B) Western blot analysis of HAUbVME-labeled HFF cell lysates, obtained from either uninfected or HSV-1-infected cells. Where indicated, 5 mM NEM was included prior to labeling. (C) HAUbVME-labeled HFF cell lysates, obtained from either uninfected or HSV-1-infected cells, preparatively immunopurified with anti-HA antibody, separated by SDS-PAGE and visualized by Coomassie staining.

FIGS. 2A-B are a diagram and graph, respectively, showing identification of Cys⁶⁵ as the putative active site cysteine residue of UL36^{USP}. (A) Following in situ trypsinolysis, peptide fragments were analyzed by MALDI-MS. Non-Ub-derived peptides were mapped to the HSV-1 tegument protein UL36 (gray). Peptides were identified from three main regions of the N-terminus (red, green, blue), and are indicated with black bars. Red sphere indicates the location of the active site cysteine (Cys⁶⁵) found to be modified by HAUbVME. (B) MS/MS spectrum of precursor ion m/z 1965.03 (M+H)¹⁺ identified by de novo sequencing methods to span residues 51-67 of UL36. Ions of the a, b, c, x, y and z series are indicated. Red sphere indicates Gly-GABA modification.

FIGS. 3A-C are diagrammatic representations of sequence comparisons of the UL36^{USP} sequence showing that the sequence is unique (distinguished from cellular enzymes). The UL36^{USP} sequence has key catalytic residues conserved across herpesviruses. (A) Amino acid sequences of the conserved motifs surrounding catalytically active amino acid residues (marked by black boxes) in members of 5 known families of DUBs in comparison with UL36^{USP}. (B) Amino acid alignment of the conserved region surrounding Cys⁶⁵ and His¹⁹⁹ (numbering based on HSV-1) in the UL36 homologues of the eight sequenced human α-, β- and γ-herpesvirus genomes. Black boxes indicate conserved residues and unfilled boxes indicate similar residues. Residues that may directly contribute to enzymatic proteolysis are indicated with arrows. (C) Plot of sequence similarity amongst the UL36 homologues of the eight sequenced human α-, β- and γ-herpesvirus genomes. The region of catalytic activity, UL36^{USP} is indicated with a bracket. The x-axis represents each position, N, of HSV-1 UL36. The y-axis represents sequence similarity calculated on a per-residue basis. Values at each residue are assigned a value for identity (1), similarity (0.5), weak similarity (0.2) or no similarity (0) to calculate a mean similarity for each amino acid position. For each position, N, the calculated mean similarity of all positions from position N−15 to position N+15 is used to determine an average mean similarity for each position that is plotted.

FIGS. 4A-B are photographs showing that UL36^{USP} is targeted only by Ub-derived probes. (A) HFFs were harvested and lysed 24 hours after infection with HSV-1. Lysates were incubated with the Ub-derived probes, HAUbVME and HAUbVS. Total lysates were separated by SDS-PAGE and immunoblotting was performed using anti-HA antibody. (B) HFFs were harvested and lysed 24 hours after infection of HSV-1, and total lysates were incubated with HAUbVME, HA-SUMO1-VME; HA-ISG15-VME, or FLAG-Nedd8-VS. Lysates were separated by SDS-PAGE and immunoblotting was performed using either anti-HA or anti-FLAG antibodies. The band marked with an asterisk (*) in the anti-FLAG immunoblot is UCH-L3, a known Nedd8 protease.

FIGS. 5A-B are line graphs, and FIGS. 5C-D are photographs showing that recombinant UL36^{USP} is an isopeptidase capable of cleaving branched Ub derivatives and Lys⁴⁸-linked polyUb chains. (A) Sub-picomole quantities (˜0.1 pmol) of His₆UL36^{USP} (blue) and His₆UL36^{USP/C65A} (red) were incubated with Ub-AMC, and hydrolysis was measured over time by the increase in AMC fluorescence. Ordinate: picomoles of Ub-AMC hydrolyzed; abscissa: elapsed time in seconds after Ub-AMC addition. (B) His₆UL36^{USP} (˜0.1 pmol) was pre-incubated for 30 minutes with either UbVME (red), Nedd8-VS (green), ISG15-VME (blue) or SUMO-1-VME (black) inhibitors. Following subsequent addition of UbAMC, activity was determined by measuring AMC fluorescence. Ordinate: picomoles of Ub-AMC hydrolyzed; abscissa: elapsed time in seconds after Ub-AMC addition. Data in (A) and (B) represent the mean of three independent experiments. (C) Biotinylated branched peptide conjugates of Ub, Nedd8, ISG15 and SUMO-1 were incubated with His₆UL36^{USP} and deconjugation of Ub/UbL proteins was determined by tris-tricine SDS-PAGE and immunoblotting with streptavidin-HRP. Loss of the biotin signal indicates hydrolysis of the isopeptide bond with concomitant release of the biotinylated peptide. UCH-L3 was used as a positive control for Ub and Nedd8 conjugates; USP5 was used as a positive control for Ub and ISG15 conjugates; and the catalytic core of Senp2 was used as a positive control for the SUMO-1 conjugate. (D) His₆UL36^{USP} was incubated with His-tagged Lys⁴⁸- or Lys⁶³-linked polyubiquitin, and chain depolymerization was evaluated at the indicated time points. USP5 was used as a positive control for polyubiquitin chain cleavage. Samples were separated by tris-tricine SDS page and immunoblotting was performed using anti-His antibody.

FIGS. 6-7 is a series photographs showing the results of a screen for inhibitors of the Herpes deubiquitinating enzyme. The photographs show the results of Western blot assays: anti-HA blots for detecting UL36 bound to the HA tagged Ub-VME probe. When the HA band decreases or disappears, the data indicates that the enzyme has bound to the inhibitor instead and can no longer be tagged. Inhibitors (Z-Z-VA-L-A-cmk, Z-VA-L-A-fink, Z-VAG-cmk, and Z-AA-L-A-fmk) specifically inhibited UL36 and did not inhibit other enzymes such as UCH-1, UCH-3, USP7, Cezanne, CYLD, and Ataxin-3.

FIG. 8 is a series of chemical structures of exemplary UL36 enzyme inhibitors.

FIG. 9 is a series of photographs showing a titration of inhibitory binding of Z-VAA-fmk and Z-AAA-fmk inhibitors to UL36 enzyme.

FIG. 10 is a diagram of a sequence alignment showing key catalytic residues and secondary structure of a Ubiquitin specific protease are conserved in three herpesvirus subfamilies. Sequence alignment of UL36 of HSV-1 (a-herpesvirus subfamily) and its MCMV (b) and hEBV (g) homologues. Only the N-terminal portion of UL36 is shown for clarity. Identical residues are white with black background, conserved residues are framed. Putative active site residues are marked by vertical arrows. The predicted secondary structure is depicted in alignment with the primary sequence. Tubes: helix; horizontal arrow: strand; line: coil.

FIGS. 11A-B are a series of photographs showing Mouse Cytomegalovirus and Epstein-Barr virus encoded deubiquitinating enzymes.

FIGS. 12A-C are a series of bar graphs showing UbAMC hydrolysis by hEBV and mCMV. UL36 homologues of EBV and mCMV are Ubiquitin-specific cysteine proteases.

FIG. 13 is a schematic drawing showing MCMV M48 mutants.

FIG. 14 is a graph showing a comparison of a multistep growth curve in an MCMV M48 mutant as compared to a wild-type strain.

FIG. 15 is a histogram showing in vivo organ titers of MCMV mutants.

FIG. 16 is a western blot analysis showing cleavage of Ub conjugates by a DUB produced by a recombinant MCMV M48 strain.

### DETAILED DESCRIPTION

The involvement of the ubiquitin-proteasome system in the life cycle of viruses includes a role in budding, cell cycle regulation and viral gene transcription. This is exemplified by herpes simplex virus 1 (HSV-1) and the discovery of USP7, also known as herpesvirus-associated USP. USP7 is a host-derived USP that binds to the ICP0 protein of HSV-1 and the EBNA1 protein of Epstein-Barr virus. USP7 associates with Mdm2 to regulate the turnover of p53, and plays a role in viral gene expression.

Based on the precedent that herpesviruses interact with at least one mammalian host DUB, modulations in DUB activity caused by infection with HSV-1 were investigated. To do so, we made use of electrophilic derivatives of Ub that were developed as probes to detect these proteases. These probes consist of epitope-tagged, electrophilic Ub derivatives that allow covalent adduct formation with DUBs. The C-terminal electrophiles specifically target the active site cysteine residue of a large number of DUBs, resulting in a covalent thioether linkage to the protease active site. An N-terminal hemagglutinin (HA) epitope tag facilitates retrieval and detection of proteases modified in this way. These probes act in a mechanism-based manner and can, therefore, react with DUBs that would escape identification by homology-based sequence alignments. Using this approach, a new family of Ub-specific cysteine proteases unique to herpesviruses was discovered in HSV-1.

An HSV-1 member of this ubiquitin-specific cysteine protease family is encoded within the N-terminal ˜500 residues of the UL36 gene product, the largest (3164 aa) tegument protein of herpes simplex virus 1 (HSV-1). Enzymatic activity of this fragment, UL36^{USP}, is detectable only after cleavage of UL36^{USP} from full-length UL36 and occurs late during viral replication. UL36^{USP} bears no homology to known deubiquitinating enzymes (DUBs) or ubiquitin binding proteins. Sequence alignment of the large tegument proteins across the family Herpesviridae indicates conservation of key catalytic residues amongst these viruses. Recombinant UL36^{USP} exhibits hydrolytic activity toward Ub-AMC, and ubiquitinated branched peptides in vitro. In addition, recombinant UL36^{USP} can cleave polyubiquitin chains, and appears to be specific for Lys⁴⁸ linkages. Mutation of the active site cysteine residue (Cys⁶⁵) to alanine abolishes this enzymatic activity. The lack of homology between UL36^{USP} and eukaryotic DUBs makes this new family of herpesvirus ubiquitin-specific proteases attractive targets for selective inhibition.

HSV DUB Polypeptides

As used herein, an HSV DUB polypeptide or protein includes a protein or peptide derived from the UL36 gene product of the human HSV-1 protein having ubiquitin-specific cysteine protease family, or derived from the gene product corresponding to the HHSV-1 UL36 gene product in another member of the herpesviridae family (such as the M48 homologue in MMCV or the BPLF1 homolog in EBV). Examples of HSV DUB polypeptides or proteins include polypeptides or proteins include amino acids 1 to 533 of the UL36 gene product from HHV HSV1, amino acids 1 to 185 of the MMCV M48 homolog, and amino acids 1-205 of the EBV BPLF1 homolog, or fragments of these proteins that retain ubiquitin-specific protease activity. In some embodiments, the HSV DUB polypeptide includes amino acids 51 to 67 of the UL36 gene product from the HSV1 polypeptide.

In one aspect, the invention provides an isolated polypeptide having deubiquitinating activity, wherein the polypeptide comprises between 200 and 1000 amino acids of the N terminal portion of a herpes virus UL36 polypeptide. In one embodiment the polypeptide is between 200 and 300 amino acids long. In one embodiment the polypeptide is between 200 and 500 amino acids long. The sequences of representative UL36 polypeptides from a number of different herpesviruses are provided in the Figures. Other UL36 sequences are known in the art and are readily obtained from public databases such as GenBank. “Isolated”, as used herein, is understood to mean that the material referred to is separated from one or more substances with which it exists in nature (e.g., is separated from cellular material, separated from other nucleic acids or polypeptides), is otherwise removed from its natural environment, and/or is produced by a process that involves the hand of man such as in vitro transcription or translation, recombinant DNA technology, chemical synthesis, etc. An isolated polypeptide may have undergone a single purification step or multiple purification steps. In certain embodiments of the invention, an isolated polypeptide is purified to greater than 90% by weight of polypeptide in a composition.

Also provided are isolated polypeptides at least 50%, at least 60%, at least 70%, at least 80%, at least 90%, at least 95%, e.g., at least 97-98% identical to a naturally occurring polypeptide having deubiquitinating activity, wherein the naturally occurring polypeptide comprises between 200 and 1000 amino acids of the N terminal portion of a herpes virus UL36 polypeptide. In one embodiment the isolated polypeptide is between 200 and 300 amino acids long. In one embodiment the isolated polypeptide is between 200 and 500 amino acids long. In certain embodiments the isolated polypeptide is identical to the naturally occurring polypeptide at the residues important for catalytic activity. In one embodiment the isolated polypeptide comprises a Cys box having a sequence selected from SEQ ID NOs: 1, 2, 5, 6, 7, 8, 9, 10, 11, and 12. In one embodiment the isolated polypeptide comprises a Cys box having a sequence at least 80% identical to SEQ ID NO: 1, 2, 5, 6, 7, 8, 9, 10, 11, or 12. In one embodiment, the isolated polypeptide comprises a His box having a sequence selected from SEQ ID NO: 3, 4, 13, 14, 15, 16, 17, 18, 19, and 20. In one embodiment, the isolated polypeptide comprises a His box having a sequence at least 80% identical SEQ ID NO: 3, 4, 13, 14, 15, 16, 17, 18, 19, and 20. In one embodiment the isolated polypeptide comprises a Cys box having a sequence at least 90% identical to SEQ ID NO: 1, 2, 5, 6, 7, 8, 9, 10, 11, or 12. In one embodiment, the isolated polypeptide comprises a His box having a sequence selected from SEQ ID NO: 3, 4, 13, 14, 15, 16, 17, 18, 19, and 20. In one embodiment, the isolated polypeptide comprises a His box having a sequence at least 90% identical SEQ ID NO: 3, 4, 13, 14, 15, 16, 17, 18, 19, and 20.

In certain embodiments of any of the inventive polypeptides, the Cys box and the His box are derived from the same herpesvirus. In other embodiments of any of the inventive polypeptides, the Cys box and the His box are derived from different herpesviruses. In certain embodiments of the invention the herpesvirus is one capable of infecting human cells. In certain embodiments of the invention the herpesvirus is selected from HHV-1, HHV-2, HHV-3, HHV-4, HHV-5, HHV-6, HHV-7, and HHV-8.

Any of the variants of an HSV DUB can be tested to determine whether they exhibit ubiquitin specific protease activity as described in the Examples.

The HSV DUB polypeptide can be obtained from any member of the Herpesvirdae family. In some embodiments, the virus is, e.g., herpes simplex virus type-1 (HSV-1), herpes simplex virus type-2 (HSV-2), cytomegalovirus (CMV), varicella-zoster virus (VSV), Epstein-Barr virus, herpesvirus-6 (HHV-6), herpesvirus-7), herpesvirus-8 (HHV-8), pseudorabies virus (PRV) and rhinotracheitis.

HSV DUB Polynucleotides

In another aspect, the invention provides an isolated nucleic acid sequence encoding a polypeptide having deubiquitinating activity, wherein the polypeptide comprises between 200 and 1000 amino acids of the N terminal portion of a herpesvirus UL36 polypeptide. In one embodiment the encoded polypeptide is between 200 and 300 amino acids long. In one embodiment the encoded polypeptide is between 200 and 500 amino acids long. In certain embodiments the encoded polypeptide is a portion of a UL36 polypeptide that lacks between 1 and 50 of the N-terminal amino acids of the UL36 polypeptide. For example, the polypeptide may lack the first 1-5, 5-10, 10-15, 15-20, or 20-25 amino acids of the UL36 polypeptide. In certain embodiments the encoded polypeptide is between 40 and 60 kD in molecular weight, e.g., between 45-50 kD in molecular weight. In certain embodiments of the invention the nucleic acid sequence is a portion of a naturally occurring herpes virus nucleic acid sequence. In other embodiments of the invention the nucleic acid sequence is one that differs from the naturally occurring sequence as a result of the degeneracy of the genetic code.

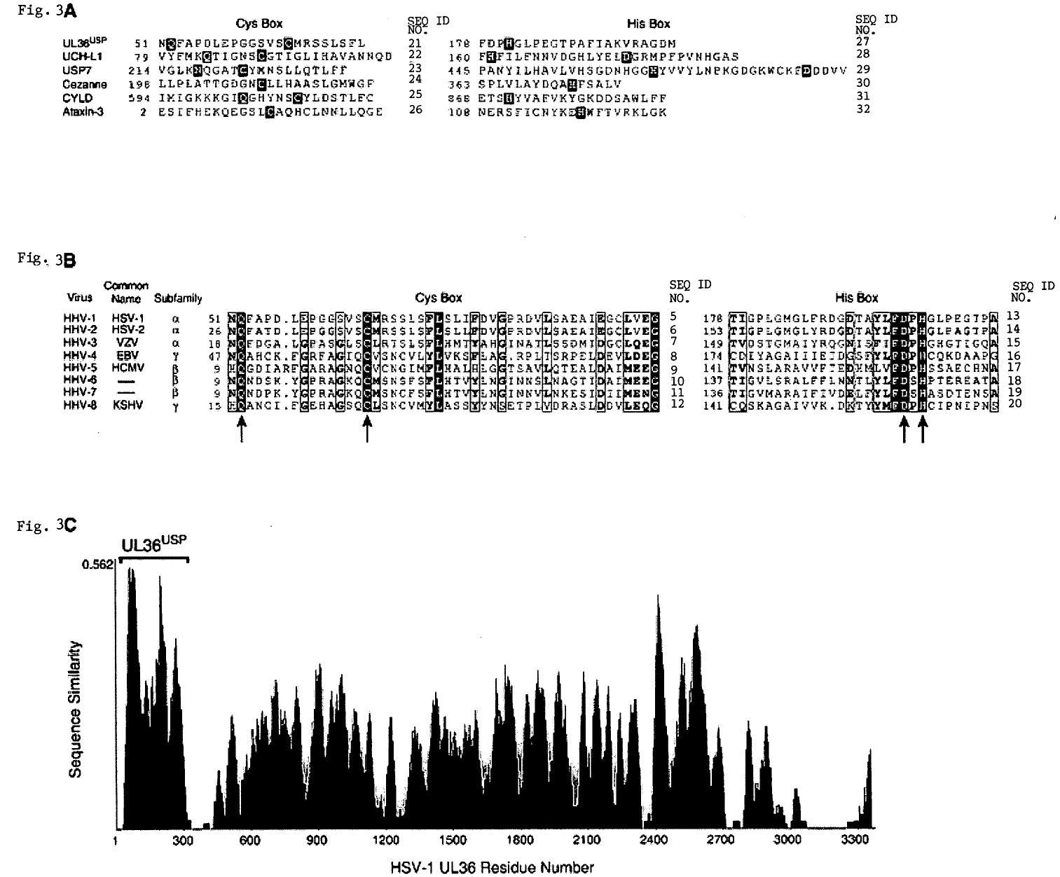

Nucleic acid sequence information is available for multiple members of the herpesviridae family, including eight members of the Herpesviridae family that have been isolated from humans (Weir, Virus Genes 16:85-93, 1998). This information, along with the amino acid sequence information disclosed herein (see, e.g., Table 1, Table 2, and FIGS. 3A-3B) and/or the known degeneracy of the genetic code, allow the artisan to make and use the HSV DUB nucleic acids discussed herein.

In another aspect, the invention provides an expression vector comprising any of the afore-mentioned nucleic acid sequences operably linked to regulatory signals suitable to direct expression in prokaryotic cells. In another aspect, the invention provides an expression vector comprising any of the afore-mentioned nucleic acid sequences operably linked to transcriptional regulatory sequences (e.g., promoters, terminators, etc.) suitable to direct expression in eukaryotic cells. Also provided are methods of producing the polypeptide comprising maintaining the cells under conditions in which the nucleic acid is expressed. A large number of suitable promoters, enhancers, terminators, and expression vectors are known in the art. Further provided are prokaryotic and eukaryotic host cells comprising the expression vector. Further provided are prokaryotic and eukaryotic host cells that express the polypeptide, wherein the cells are not infected with a herpesvirus.

Anti-Sense RNA

Another aspect of the invention pertains to isolated antisense nucleic acid molecules that are hybridizable to or complementary to nucleic acid molecule encoding an HSV DUB polypeptide, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire HSV DUB coding strand, or to only a portion thereof. Nucleic acid molecules encoding fragments, homologs, derivatives and analogs of an HSV DUB protein, or antisense nucleic acids complementary to an HSV DUB nucleic acid sequence are additionally provided.

In one embodiment, an antisense nucleic acid molecule is antisense to a “coding region” of the coding strand of a nucleotide sequence encoding HSV DUB. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding HSV DUB. The term “noncoding region” refers to 5′ and 3′ sequences which flank the coding region that are not translated into amino acids (i.e., also referred to as 5′ and 3′ untranslated regions).

Antisense nucleic acids for an HSV DUB protein can be designed according to the rules of Watson and Crick or Hoogsteen base pairing. The antisense nucleic acid molecule can be complementary to the entire coding region of HSV DUB mRNA, but more preferably is an oligonucleotide that is antisense to only a portion of the coding or noncoding region of HSV DUB mRNA. For example, the antisense oligonucleotide can be complementary to the region surrounding the translation start site of HSV DUB mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a HSV DUB protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual-.beta.-units, the strands run parallel to each other (Gaultier et al. (1987) Nucleic Acids Res 15:6625 6641). The antisense nucleic acid molecule can also comprise a 2′-o-methylribonucleotide (Inoue et al. (1987) Nucleic Acids Res 15: 6131 6148) or a chimeric RNA-DNA analogue (Inoue et al. (1987) FEBS Lett 215: 327 330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585 591)) can be used to catalytically cleave HSV DUB mRNA transcripts to thereby inhibit translation of HSV DUB mRNA. A ribozyme having specificity for an HSV DUB-encoding nucleic acid can be designed based upon the nucleotide sequence of an HSV DUB cDNA. For example, a derivative of a Tetrahymena L-19 IVS RNA can be constructed in which the nucleotide sequence of the active site is complementary to the nucleotide sequence to be cleaved in a HSV DUB-encoding mRNA. See, e.g., Cech et al. U.S. Pat. No. 4,987,071; and Cech et al. U.S. Pat. No. 5,116,742. Alternatively, HSV DUB mRNA can be used to select a catalytic RNA having a specific ribonuclease activity from a pool of RNA molecules. See, e.g., Bartel et al., (1993) Science 261:1411 1418.

Alternatively, HSV DUB gene expression can be inhibited by targeting nucleotide sequences complementary to the regulatory region of the HSV DUB (e.g., the HSV DUB promoter and/or enhancers) to form triple helical structures that prevent transcription of the HSV DUB gene in target cells. See generally, Helene, (1991) Anticancer Drug Des. 6: 569 84; Helene, et al. (1992) Ann. N.Y. Acad. Sci. 660:27 36; and Maher (1992) Bioassays 14: 807 15.

In various embodiments, the HSV DUB nucleic acids of can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5 23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670 675.

PNAs of HSV DUB can be used in therapeutic and diagnostic applications. For example, PNAs can be used as antisense or antigene agents for sequence-specific modulation of gene expression by, e.g., inducing transcription or translation arrest or inhibiting replication. PNAs of HSV DUB can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of HSV DUB can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of HSV DUB can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357 63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973 88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119 11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553 6556; Lemaitre et al., 1987, Proc. Natl. Acad. Sci. 84:648 652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958 976) or intercalating agents. (See, e.g., Zon, 1988, Pharm. Res. 5: 539 549). To this end, the oligonucleotide may be conjugated to another molecule, e.g., a peptide, a hybridization triggered cross-linking agent, a transport agent, a hybridization-triggered cleavage agent, etc.

HSV DUB RNAi

Also provided by the invention are oligonucleotide agents that interfere with translation of HSV DUB mRNA. RNA interference (RNAi) is one type of gene silencing in which duplex RNA, either endogenous to cells or delivered exogenous to the cells, interferes with the function of an exogenous or an endogenous gene through a complex form of hybridization to and cleavage of a target mRNA transcript. Preferably the HSV DUB interfering RNA do not induce interferon. An example of such an RNAi is a double-stranded nucleic acids less than 25 nucleotides (nt). In some embodiments, the HSV DUB RUNAi are 21-23 nt dsRNA (siRNAs) having overhanging 3′ ends and which mediate sequence-specific mRNA degradation.

In one aspect, the invention provides methods of treating a subject suffering from or susceptible to a HSV-related disease by administering to a subject in need thereof an RNAi inducing entity that induces an RNA that antagonizes expression or function of an HSV DUB RNA.

In one embodiment, the RNAi inducing entity is an RNAi construct. In another embodiment, the RNAi inducing entity is a small-interfering RNA (siRNA), wherein the siRNA is 19-30 base pairs long. In a related embodiment, the RNAi construct is an expression vector having a coding sequence that is transcribed to produce one or more transcriptional products that produce siRNA in the treated cells. In another related embodiment, the RNAi construct is a hairpin RNA which is processed to an siRNA in the treated cells, wherein the RNAi construct attenuates one or more HSV DUB genes.

According to certain embodiments, the RNAi construct attenuates expression of a gene resulting in reducing proliferation. In one embodiment, the RNAi construct is an expression vector having a coding sequence that is transcribed to produce one or more transcriptional products that produce siRNA in the treated cells.

HSV DUB Antibodies

The invention also includes antibodies specifically reactive with an HSV DUB antibody. Anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal such as a mouse, a hamster or rabbit can be immunized with an immunogenic form of the peptide. Techniques for conferring immunogenicity on a protein or peptide include conjugation to carriers, or other techniques, well known in the art.

An immunogenic portion of HSV DUB can be administered in the presence of an adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of HSV DUB. In yet a further preferred embodiment of the present invention, the anti-HSV DUB antibodies do not substantially cross react (i.e. react specifically) with a protein which is e.g., less than 80 percent homologous to a protein with one or more of the amino acid sequence SEQ ID NOs. 5-20, preferably less than 90 percent homologous with a protein with one or more of the amino acid sequence SEQ ID NOs. 5-20; and, most preferably less than 95 percent homologous with a protein with one or more of the amino acid sequence SEQ ID NOs. 5-20. By “not substantially cross react”, it is meant that the antibody has a binding affinity for a non-homologous protein which is less than 10 percent, more preferably less than 5 percent, and even more preferably less than 1 percent, of the binding affinity for a protein of a protein with one or more of the amino acid sequence SEQ ID NOs. 5-20.

The term antibody as used herein is intended to include fragments thereof which are also specifically reactive with HSV DUB. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibodies of the present invention are further intended to include biospecific and chimeric molecules having anti-HSV DUB. Thus, both monoclonal and polyclonal antibodies (Ab) directed against HSV DUB, and antibody fragments such as Fab′ and F(ab′)₂, can be used to block the action of HSV DUB.

Various forms of antibodies can also be made using standard recombinant DNA techniques. (Winter and Milstein, Nature 349: 293 299 (1991) specifically incorporated by reference herein.) For example, chimeric antibodies can be constructed in which the antigen binding domain from an animal antibody is linked to a human constant domain (e.g. Cabilly et al., U.S. Pat. No. 4,816,567, incorporated herein by reference). Chimeric antibodies may reduce the observed immunogenic responses elicited by animal antibodies when used in human clinical treatments.

In addition, recombinant “humanized antibodies” which recognize HSV DUB can be synthesized. Humanized antibodies are chimeras comprising mostly human IgG sequences into which the regions responsible for specific antigen-binding have been inserted. Animals are immunized with the desired antigen, the corresponding antibodies are isolated, and the portion of the variable region sequences responsible for specific antigen binding are removed. The animal-derived antigen binding regions are then cloned into the appropriate position of human antibody genes in which the antigen binding regions have been deleted. Humanized antibodies minimize the use of heterologous (i.e. inter species) sequences in human antibodies, and thus are less likely to elicit immune responses in the treated subject.

Construction of different classes of recombinant antibodies can also be accomplished by making chimeric or humanized antibodies comprising variable domains and human constant domains (CH1, CH2, CH3) isolated from different classes of immunoglobulins. For example, antibodies with increased antigen binding site valencies can be recombinantly produced by cloning the antigen binding site into vectors carrying the human: chain constant regions. (Arulanandam et al., J. Exp. Med., 177: 1439 1450 (1993), incorporated herein by reference.)

In addition, standard recombinant DNA techniques can be used to alter the binding affinities of recombinant antibodies with their antigens by altering amino acid residues in the vicinity of the antigen binding sites. The antigen binding affinity of a humanized antibody can be increased by mutageneesis based on molecular modeling. (Queen et al., Proc. Natl. Acad. Sci. 86: 10029 33 (1989) incorporated herein by reference.

Identifying Modulators of HSV DUB Polypeptides

The invention provides a method of identifying a compound that modulates activity of an HSV DUB polypeptide comprising: (a) contacting the HSV DUB polypeptide according with a test compound; and (b) monitoring for modulation of HSV DUB activity, wherein a compound which modulates HSV DUB activity is thereby identified. In one embodiment the activity that is monitored is protease activity. In one embodiment the activity that is monitored is dubiquitinating activity. In one embodiment the monitoring comprises detecting cleavage of a synthetic ubiquitinated substrate. In certain embodiments the method is used to identify a compound that inhibits HSV DUB activity.

In another aspect, the invention provides a method of identifying an agent which inhibits activity of a DUB enzyme. The method includes introducing into a host cell a first nucleic acid construct encoding a fusion protein comprising the DUB enzyme and a second protein which is an enzyme; and a second nucleic acid construct encoding a ubiquitin conjugated substrate of the second protein, thereby producing a host cell containing the first and second constructs. The host cell so produced is maintained under conditions appropriate for expression of the fusion protein and the ubiquitin-conjugated substrate, thereby producing a host cell expressing the fusion protein and the ubiquitin-conjugated substrate. The host cell is then combined with an agent to be assessed and the extent to which the enzyme in the fusion protein acts upon the ubiquitin-conjugated substrate is determined. The agent is an inhibitor of the DUB enzyme if the DUB enzyme acts to a lesser extent on the ubiquitin-conjugated substrate in the presence of the agent than in the absence of the agent. In some embodiments, the DUB enzyme in the fusion protein cleaves the ubiquitin-conjugated substrate and the extent to which the DUB enzyme acts is assessed by determining the extent to which cleavage of the ubiquitin-conjugated substrate occurs.

The test compounds can be, e.g., small molecules, polypeptides, nucleic acids, etc. They can be natural products, members of a combinatorial library, etc. In one embodiment the method comprises contacting the polypeptide with a plurality of test compounds, e.g., in individual vessels or wells. In one embodiment the test compounds are alpha-halogenated methyl ketones, and the method comprises selecting an alpha-halogenated methyl ketone that has greater ability to inhibit activity than most (e.g., more than 80%) or all other alpha-halogenated methyl ketones being tested. In one embodiment the test compounds are cysteine protease inhibitors, and the method comprises selecting a cysteine protease inhibitor that has greater ability to inhibit activity than most (e.g., more than 80%) or all other cysteine protease inhibitors being tested. One of skill in the art will be aware of many cysteine protease inhibitors that can be tested according to the methods. The HSV DUB polypeptide may be from any herpesvirus, or can be a variant of a naturally occurring HSV DUB polypeptide such as those described above, having DUB activity.

Further provided are a machine readable storage medium which comprises the results obtained from such a method.

In one embodiment, the method comprises generating a predicted dimensional structure for the HSV DUB polypeptide and using a computer based system to identify candidate compounds predicted to bind to the HSV DUB polypeptide. A number of computer-based docking programs useful for the practice of the method are known to those of skill in the art. Also available are structures of numerous candidate compounds suitable for docking. Further provided is a machine readable storage medium encoded with data providing coordinates of a predicted three dimensional structure of the HSV DUB or equivalent information. The storage medium is capable of displaying on a computer screen or similar viewing device, a three-dimensional graphical representation of a molecule or molecular complex which comprises the HSV DUB and, optionally, a candidate compound docked thereto.

Pharmaceutical Compositions

The invention provides a pharmaceutical composition comprising a compound identified according to the methods of the invention. One of skill in the art would recognize that a compound identified using the methods described herein can be formulated as a pharmaceutical composition together with any appropriate pharmaceutically acceptable carrier and administered by a variety of routes. Pharmaceutically acceptable carriers are known in the art and include, e.g., aqueous solutions such as water, physiologically buffered saline or other solvents or vehicles such as glycols, glycerol, oils, etc. Additional pharmaceutically acceptable compounds such as excipients, stabilizers, preservatives, absorption enhancers, etc., and formulated for delivery by any available route including, but not limited to parenteral, oral, by inhalation to the lungs, nasal, bronchial, ophthalmic, transdermal (topical), transmucosal, buccal, rectal, and vaginal routes. Supplementary active agents, e.g., agents independently active against the disease or clinical condition to be treated, or agents that enhance activity of a compound, can also be incorporated into the compositions.

One of skill in the art would recognize that an effective amount of the pharmaceutical composition is administered to a subject by any suitable route of administration including, but not limited to, intravenous, intramuscular, by inhalation, by catheter, intraocularly, orally, rectally, intradermally, intra-articularly, intrathecally, by application to the skin, etc.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are typically preferred. The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Exemplary doses include milligram or microgram amounts of the inventive compounds per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses depend upon the potency of the agent, and may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, the severity of the disease, disorder, or condition, etc. While the medical treatment and medicine is particularly useful for inhibiting herpes and other infectious diseases in persons (human beings) (homo sapiens), they can also be useful for veterinary purposes for treating viral and bacterial infections and infectious diseases in animals, such as: dogs, cats, birds, horses, cows, sheep, swine (pigs and hogs), and other farm animals, as well as rodents and other animals seen in zoos.

Manipulating Ubiquinated Proteins Using HSV DUB Proteins

Isolated HSV DUB, or a cellular extract containing HSV DUB produced from a recombinant DNA expression vector, can be used to cleave ubiquitin from ubiquitin-containing proteins in vitro or in vivo. The HSV DUB is contacted with a ubiquitin-containing protein under conditions that result in removal of the ubiquitin from the protein. If desired, the ubiquitin-containing proteins can be concentrated on beads, which facilitates subsequent handling and purification of the ubiquitin-containing proteins, prior to contacting the ubiquitin-containing proteins with the HSV DUB-containing protease.

For example, a cellular extract known to or suspected of containing a ubiquitinated protein or proteins can be prepared from a culture of cells (including host cells expressing a recombinant DNA expression vector (by simply concentrating and lysing the cell culture. The lysis can be followed, optionally, by various degrees of purification. The range of conditions appropriate for in vitro cleavage can be determined empirically by one skilled in the art.

In addition, HSV DUB proteases can be used to deubiquitinate fusion proteins in vivo. For example, prokaryotic cells harboring an expression vector encoding an HSV DUB protease can be transformed with an expression vector encoding a ubiquitin fusion protein. Such cells will produce a deubiquitinated product having a predetermined N-terminal amino acid residue.

In some fusions of ubiquitin to a non-ubiquitin protein or peptide, the presence of the ubiquitin moiety may inhibit or modify the functional activity of the non-ubiquitin protein or peptide. In this case, ubiquitin can be used as a temporary inhibitor (or modifier) of the functional activity of the non-ubiquitin protein or peptide, with the ability to restore the original functional activity at any desired time, either in vitro or in vivo, by contacting the corresponding ubiquitin fusion with the ubiquitin-specific protease to remove the ubiquitin moiety.

HSV DUB proteases can be provided in a kit that is used for removing ubiquitin from a desired population of molecules. The HSV proteases can be packaged in a suitable container. The kit can further comprise instructions for using the kit to remove ubiquitin from a desired population of proteins.

Other features, objects, and advantages of the invention will be apparent from the description and drawings. The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Such techniques are explained fully in the literature. Non-limiting descriptions of certain of these techniques are found in the following references: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005.

Additional embodiments are within the examples below and in the claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. Certain claims are presented in dependent form for the sake of convenience, but Applicant reserves the right to rewrite any dependent claim in independent format to include the limitations of the independent claim and any other claim(s) on which such claim depends, and such rewritten claim is to be considered equivalent in all respects to the dependent claim in whatever form it is in (either amended or unamended) prior to being rewritten in independent format.

### EXAMPLE 1

### A Deubiguitinating Enzyme Encoded by HSV-1 Belongs to a New Family of Cysteine Proteases that is Conserved Across the Family Herpesviridae

The data described in Example 1 were generated using the following materials and methods.

Cells and Viruses

Primary human foreskin fibroblasts (HFFs) and Vero cells were cultured in Dulbecco's modified essential medium (DMEM) supplemented with 10% fetal calf serum, penicillin (100 U/ml), streptomycin (100 μg/ml) and 2 mM glutamine in a humidified atmosphere at 37° C. with 5% CO₂. Virus stocks of HSV-1 KOS were prepared as described (Desai, 2000).

Electrophilic Labeling and Detection

HFFs were infected with HSV-1 KOS at an MOI of 10 and harvested by trypsinolysis at the indicated times post infection. Cells were washed 1× in PBS and pelleted by centrifugation in a microcentrifuge at 3,000×g for 20 minutes at 4° C. Cells were lysed by adding 2× the pellet volume of NP40 lysis buffer (150 mM NaCl, 50 mM Tris pH 7.4, 5 mM MgCl₂, 0.5 mM EDTA, 2 mM DTT, 2 mM ATP, 0.5% NP40) and rocking at 4° C. for 1 hour. Cell lysates were centrifuged at 12,000×g for 20 minutes at 4° C. Protein concentration of supernatants was established by Bradford assay and lysates were standardized to 5 μg/μl by the addition of the required volume of NP40 lysis buffer. For each labeling reaction, 50 μg of lysate was incubated with 0.8 μg of probe in a total volume of 60 μL of homogenization buffer (50 mM Tris pH 7.5, 5 mM MgCl₂, 0.5 mM EDTA, 2 mM DTT, 2 mM ATP, 250 mM sucrose). For reactions involving NEM, lysates were incubated at room temperature for 30 minutes with 5 mM NEM prior to addition of probe. Reactions were incubated for 30 minutes at R.T. Samples were boiled in reducing sample buffer and separated by SDS-PAGE. Proteins were transferred to PVDF membranes and immunoblotted with mouse monoclonal anti-HA antibody (12CA5) at 1:4000 dilution followed by goat anti-mouse IgG-HRP (Southern Biotech) at 1: 10,000 dilution. Blots were developed using Western Lightning chemiluminescence reagent (Perkin Elmer).

Immunopurification

HFFs were grown to 70% confluency and infected with HSV-1 KOS at an MOI of 10. Approximately 7.5×10⁷ cells were used for each immunoprecipitation. Cells were harvested at 20 hours post infection, and lysed using NP40 lysis buffer (as described above). Supernatants were diluted 1:2 in homogenization buffer and incubated with 15 μg of probe for 2 hr at R.T. SDS was added to 0.4% followed by vigorous vortexing. Samples were diluted to 0.1% SDS with homogenization buffer. Samples were then incubated with 100 μL packed volume of anti-HA (12CA5) beads and incubated overnight at 4° C. on a rocking platform. Beads were washed with NET buffer (50 mM Tris pH 7.4, 0.5% NP40, 5 mM EDTA, 150 mM NaCl) and bound protein was eluted with 100 mM glycine pH 2.5. Samples were concentrated and subjected to SDS-PAGE followed by Coomassie staining.

MALDI-TOF Analysis and Tandem Mass Spectrometry

Identification of isolated polypeptides (Coomassie stained bands) was performed as described (Kinter and Sherman, 2000). Trypsinized proteins were analyzed using a MALDI-TOF/TOF mass spectrometer (Bruker Daltonics). Proteins were identified by peptide mass fingerprinting using Mascot (Matrixscience) and Biotools version 2.1 (Bruker Daltonics). Individual peptides were sequenced by laser induced fragmentation (LIFT) methodology as described (Suckau et al., 2003).

Plasmid Construction and Purification of Proteins

The first 1599 nucleotides of UL36 were cloned by PCR and ligated into the Nhe I and Not I sites of pET-28a(+), resulting in a construct with both N and C-terminal 6-His tag fusions. Protein was expressed in either E. coli BL21 cells, or BL21 RIPL cells (Stratagene). Cultures were grown to an O.D. of 600 and induced with 1 mM IPTG (final concentration). Cells were harvested at 3 hours post-induction and lysed by the addition of 0.2 mg/ml of lysozyme in PBS. Pellets were lysed for 1 hour at 4° C. on a rocking platform. Lysates were centrifuged at 11,000 rpm for 15 minutes at 4° C. in an SA-600 rotor. Supernatants were filtered and recombinant protein purified using Ni-NTA resin. The resin was washed with a step gradient of imidazole, and purified protein was released with elution buffer (200 mM NaCl, 20 mM Tris pH 8.0, 270 mM imidazole).

Cleavage of Ub-AMC

Recombinant UL36^{USP} and UL36^{USP/C65A} (˜0.1 pmol) were incubated in 125 μl kinetics buffer (50 mM HEPES, pH 7.5, 0.5 mM EDTA) supplemented with 1 mM DTT at room temperature for 10 minutes. To this was added 75 μl of Ub-AMC (250 nM in kinetics buffer) and AMC liberation was measured at 25° C. For inhibition experiments, one of UbVME, SUMO-VME, ISG15-VS, or Nedd8-VS (10 pmol) was incubated with recombinant UL36^{USP} (˜0.1 pmol) for 30 minutes at room temperature. To this was added 75 μl of Ub-AMC (250 nM in kinetics buffer) and AMC liberation was measured. Cleavage experiments were performed in triplicate. All fluorescence measurements were made using a Wallac Victor³ 1420 fluorescence/luminescence plate reader (Perkin Elmer) using 380 nm excitation and 460 nm emission wavelengths, with 5 second intervals between measurements. Data were exported as an Excel file and plotted as mean values of replicate experiments using Kaleidagraph v3.6 (Synergy Software).

Synthesis of Branched Peptides

Branched peptide synthesis is analogous to that reported (Misaghi et al., 2005). His-Ub, ISG15, GST-SUMO-1, His-Nedd8, their respective E1 enzymes, and the E2 carriers, UbCH8, UbCH9, and UbcH12 were obtained from Boston Biochem. The general protocol for synthesis of Ub/UbL-peptide conjugates linked by isopeptide bonds is as follows: To 50 μl of conjugation buffer (100 mM Tris, pH 7.4, 5 mM MgCl₂, 20 mM DTT, 40 mM ATP) was added Ub/UbL (20 μl, ˜10 μM final) and biotinylated peptide 7-mer (biotin-VKAKIQD-OH, 20 μl of 1 mM stock, 250 μM final). The solution was mixed thoroughly and to this was added the appropriate E1 activating enzyme (50 nM final) and Ub/UbL carrier protein (250 nM final). The solution was mixed and incubated for 15 hours at 37° C. The reaction mixture was transferred to a microcentrifuge membrane filter (Vivascience, 5000 Da MWCO), diluted to 600 μl total volume with 50 mM Tris, pH 7.4 and concentrated at 4° C. to 50 μl. This dilution/concentration procedure was repeated six times. The products were transferred to a clean tube and diluted with 50 mM Tris, pH 7.4 to a final volume of 100 μl.

Isopeptidase Assays

Isopeptidase assays of Ub and UbL peptide conjugates were conducted using aliquots of conjugates synthesized as above. To 6 μl of reaction buffer (50 mM Tris, pH 7.4, 2 mM DTT) was added Ub-peptide (3 μl), Nedd8-peptide (5 μl), ISG15-peptide (3 μl) or SUMO-1-peptide (2 μl) conjugate. Additional 50 mM Tris, pH 7.4 was added to a final volume of 11 μl. Reactions with UL36^{USP} (1 μl, ˜10 nM final) were incubated at 37° C. for 12 hours. Reactions with UCH-L3 (1 μl, 70 nM final) were incubated at 37° C. for 2 hours. Reactions with USP5 (1 μl, 60 nM final) were incubated at 37° C. for 12 hours. Reactions with Senp2 (1 μl, 100 nM final) were incubated at 37° C. for 2 hours. Polyubiquitin chain assays were performed using identical enzyme quantities as described for Ub/UbL peptide conjugate assays. Reactions with UL36^{USP} were incubated at 37° C. for 12 hours. Reactions with USP5 were incubated at 37° C. for 2 hours. All reactions were terminated by addition of reducing sample buffer and separated by tris-tricine SDS-PAGE. Proteins were transferred to PVDF membrane and immunoblotting was performed using Streptavidin-HRP (Pharmacia) at 1:1000 dilution for Ub/UbL peptide conjugate assays or anti-penta-His antibody (Qiagen) at 1:3000 dilution. UCH-L3 was prepared as described (Misaghi et al., 2005); USP5, Lys⁴⁸-linked polyUb and Lys⁶³-linked polyUb were obtained from Boston Biochem.

Labeling and Identification of UL36

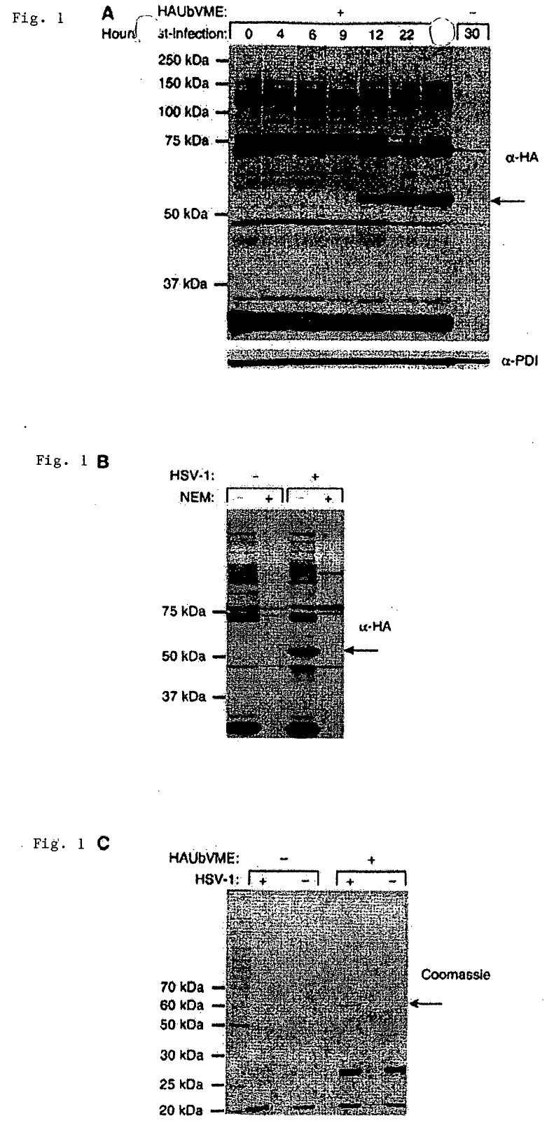

To profile DUB activity during HSV-1 infection, we used HA-tagged Ub-vinylmethyl ester (HAUbVME) (Borodovsky et al., 2002). Human foreskin fibroblasts (HFFs) were infected with HSV-1 at a multiplicity of infection of 10, harvested at 4, 6, 9, 12, 22 and 30 hours post-infection (p.i.), and lysed. Immunoblot analysis of lysates incubated with HAUbVME revealed a prominent 57 kDa polypeptide, unique to HSV-1 infected lysates (FIG. 1A, Lane 5). The labeled species is first detectable at 12 hours, and peaks at 22 hours p.i (FIG. 1A, Lane 6). Labeling with HAUbVME was inhibited by inclusion of N-ethylmaleimide (NEM), consistent with the involvement of a cysteine residue in the target enzyme (FIG. 1B, Lane 4).

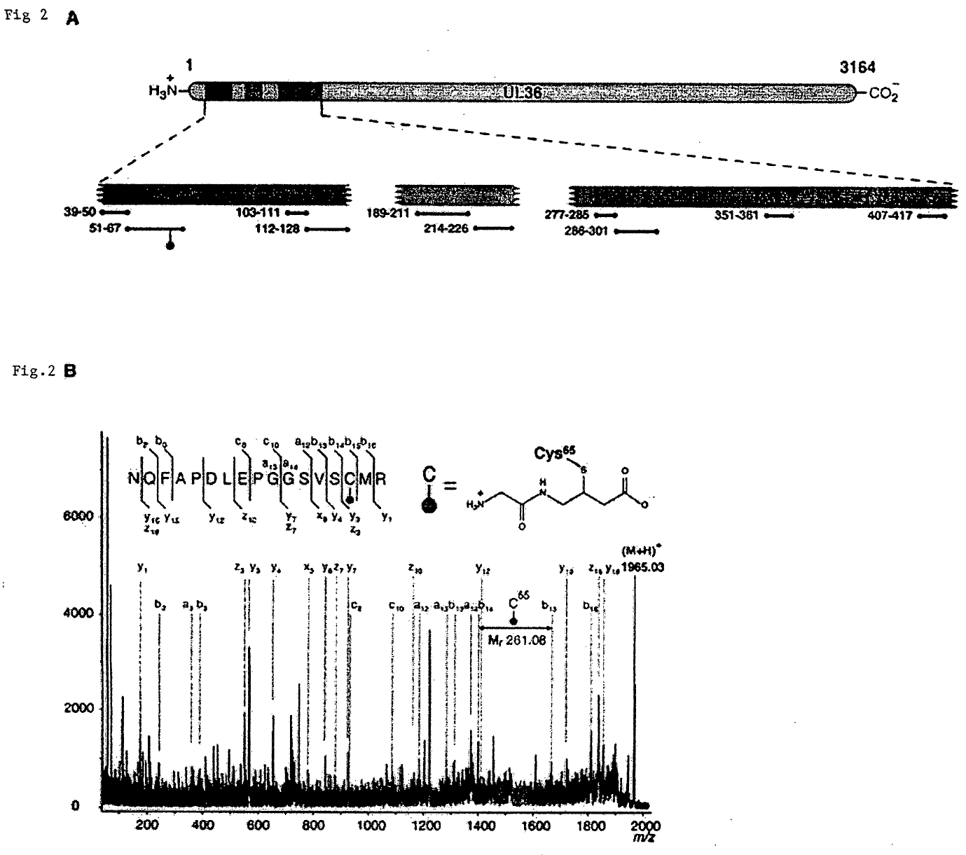

To identify the reactive protease, we performed a preparative anti-HA immunopurification on HAUbVME-treated HSV-1-infected cell lysates. Denaturing gel electrophoresis (SDS-PAGE) followed by Coomassie staining of the eluted material revealed a protein migrating at the expected relative molecular mass and present only in virus-infected cells (FIG. 1C, lane 3). After in situ trypsinolysis and tandem mass spectrometric (MS/MS) analysis, we identified five peptides derived from the Ub moiety of the probe and ten peptides from a protein that did not match any known DUB. All ten of these peptides mapped to the N-terminal 417 residues of the 3164-amino acid tegument protein, UL36, the gene product of the largest open reading frame in the α-herpesvirus, HSV-1 (FIG. 2A). Tegument proteins are part of an amorphous region between the icosahedral capsid and lipid envelope in virion particles. While the role of many herpesvirus tegument proteins remains largely unknown, the tegument layer contains a number of proteins necessary for modification of the host response to viral infection.

The relative molecular mass of the UL36 adduct with HAUbVME is 57 kDa, corresponding to the 10.3 kDa probe in covalent linkage to a ˜47 kDa fragment of UL36. As the full length UL36 open reading frame encodes a polypeptide of 3164 amino acids, the 47 kDa fragment requires either an alternative stop codon within the UL36 gene, or post-translational cleavage of full-length UL36 protein to account for its size. With the HAUbVME probe we have observed labeling of the 47 kDa UL36 fragment but never of the full-length UL36 gene product. We have been unable to confidently identify the C-terminus of this fragment from our mass spectrometry data, but our size estimates based on SDS-PAGE and the MS/MS data place the C-terminus beyond position 417, likely in the range of residues 450-500. The characterization of UL36, as reported in the literature, has shown the existence of a large polypeptide only (McNabb and Courtney, 1992). Viruses are genetically economical: post-translational cleavage of gene products to generate smaller functional proteins is common. For herpesviruses, the UL26 derived serine protease is an example of such a proteolytic conversion (Liu and Roizman, 1993). Our data do not distinguish between proteolysis, an unusual mode of termination of translation, or the presence of an unconventional UL36 derived transcript as the source of the active UL36 species. Nonetheless, its identity is securely established by mass spectrometry and by its relative molecular mass. We use the name UL36^{USP} to refer to the N-terminal region of UL36 that displays USP-type activity, as will be further demonstrated below.

Identification of the Nucleophilic Cysteine Residue by MS/MS

We used mass spectrometry to identify the site of modification of UL36^{USP} by the HAUbVME probe. Following trypsinolysis of the adduct between a DUB and HAUbVME, peptides that contain the active site cysteine are modified with a glycine-γ-aminobutyrate (Gly-GABA) group in a β-thioether linkage. A single peptide, mapping to residues 51-67, matched the mass of an expected tryptic fragment of UL36 with the calculated 159 Da increase attributable to Gly-GABA addition. Tandem MS (MS/MS) sequencing of this peptide confirmed its identity and established the site of modification as Cys⁶⁵ (FIG. 2B).

Homologs of UL36

A BLAST search (Altschul et al., 1990) of the N-terminal region of HSV-1 UL36 yields only homologous tegument proteins from other herpesviruses and no matches to known DUBs (FIG. 3A). All members of the herpesvirus family possess a UL36 homolog with sequence similarity to HSV-1 UL36. Alignment of UL36 homologs from the eight sequenced human α-, β- and γ-herpesvirus genomes shows absolute conservation of Gln⁵², Cys⁶⁵, Leu⁷⁰, Gly⁹⁷, Phe¹⁹⁶, Asp¹⁹⁷ and His¹⁹⁹ (numbering based on HSV-1 UL36) (FIG. 3B). Of these amino acids, Cys⁶⁵ and His¹⁹⁹ represent the minimal residues for catalysis by a cysteine protease, with Gln⁵² and Asp¹⁹⁷ as likely contributors to the proteolytic mechanism. The strict conservation of Cys⁶⁵ is striking in light of the observation that this residue is the site of modification by HAUbVME, identifying it as the active site nucleophile. Although the sequences of full-length HSV-1 UL36 and its homologs are quite diverse across herpesviruses, the N-terminus contains a number of residues conserved or similar among them (FIG. 3C). The fact that absolute conservation is seen for residues that could directly contribute to catalytic proteolysis supports the notion that the N-terminal region of UL36 plays a key role in herpesvirus biology. No obvious similarity is evident when comparing the sequence motifs surrounding Cys⁶⁵ and His¹⁹⁹ of UL36 with known mammalian DUBs or known cysteine protease families in general. We propose that the protease encoded by the UL36 gene of HSV-1, homologous to the large tegument proteins of other herpesviruses, is the founding member of a new family of cysteine proteases.

Substrate Specificity of UL36

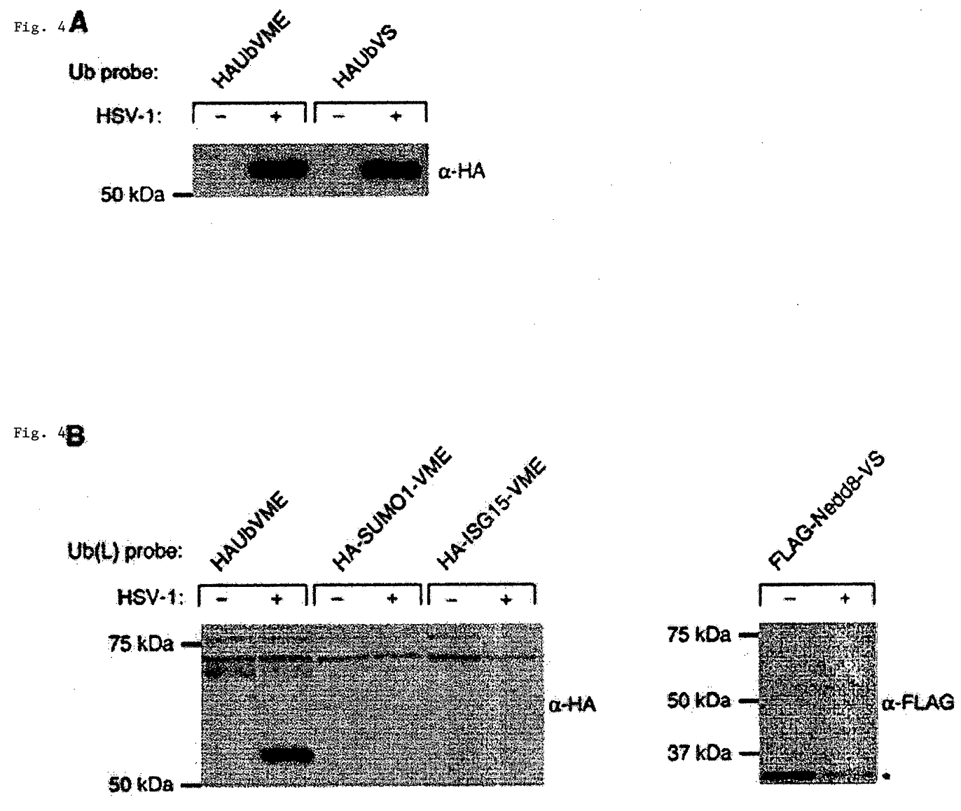

To address whether UL36^{USP} labeling by HAUbVME was a consequence of the Ub or the VME moiety, we incubated lysates of HSV-infected HFFs at 24 hours p.i. with related electrophilic derivatives (Borodovsky et al., 2002). We observed comparably robust labeling of UL36^{USP} by HAUb-vinylsulfone (HAUbVS) (FIG. 4A), as well as a number of other probes (data not shown). Reaction between these Ub-derived inhibitors and UL36^{USP} is therefore controlled by the Ub moiety rather than by the identity of the electrophile. There are a number of Ub-like (UbL) proteins in eukaryotes (Larsen and Wang, 2002). This family of UbL proteins has diversified functionally to cover many cellular pathways: SUMO proteins function in nuclear signaling and transcription (Hay, 2005); ISG15 is an interferon-inducible gene product (Ritchie and Zhang, 2004); and the closest homolog of Ub, Nedd8, is an activator of the cullin family of E3 Ub ligases (Petroski and Deshaies, 2005; Willems et al., 2004). Probes derived from Ub and UbL modifiers target proteases that are either specific for a single Ub/UbL or selective for a limited number of these proteases (Borodovsky et al., 2002; Hemelaar et al., 2004). We explored the reactivity of UL36^{USP} with Ub and UbL-derived probes to determine its specificity. We incubated lysates of mock-infected and HSV-infected HFFs at 24 hours p.i. with either HAUbVME or with one of three UbL probes, HA-SUMO-1-VME, HA-ISG15-VME or FLAG-Nedd8-VS (FIG. 4B). We did not observe reaction of UL36^{USP} with the HA-SUMO-1-VME (12.8 kDa), HA-ISG15-VME (18.8 kDa) or FLAG-Nedd8-VS (9.7 kDa) probes by immunoblot, nor did we observe any other labeled polypeptides specific to infected lysates. Each of these UbL probes labels high molecular weight species that are known UbL proteases (Hemelaar et al., 2004). These results establish the existence of an HSV-1 protease encoded by the N-terminal region of the UL36 gene product that is capable of recognizing, binding, and reacting with electrophilic Ub, but not UbL, derivatives.

Cloning and Expression of Active UL36^{USP}

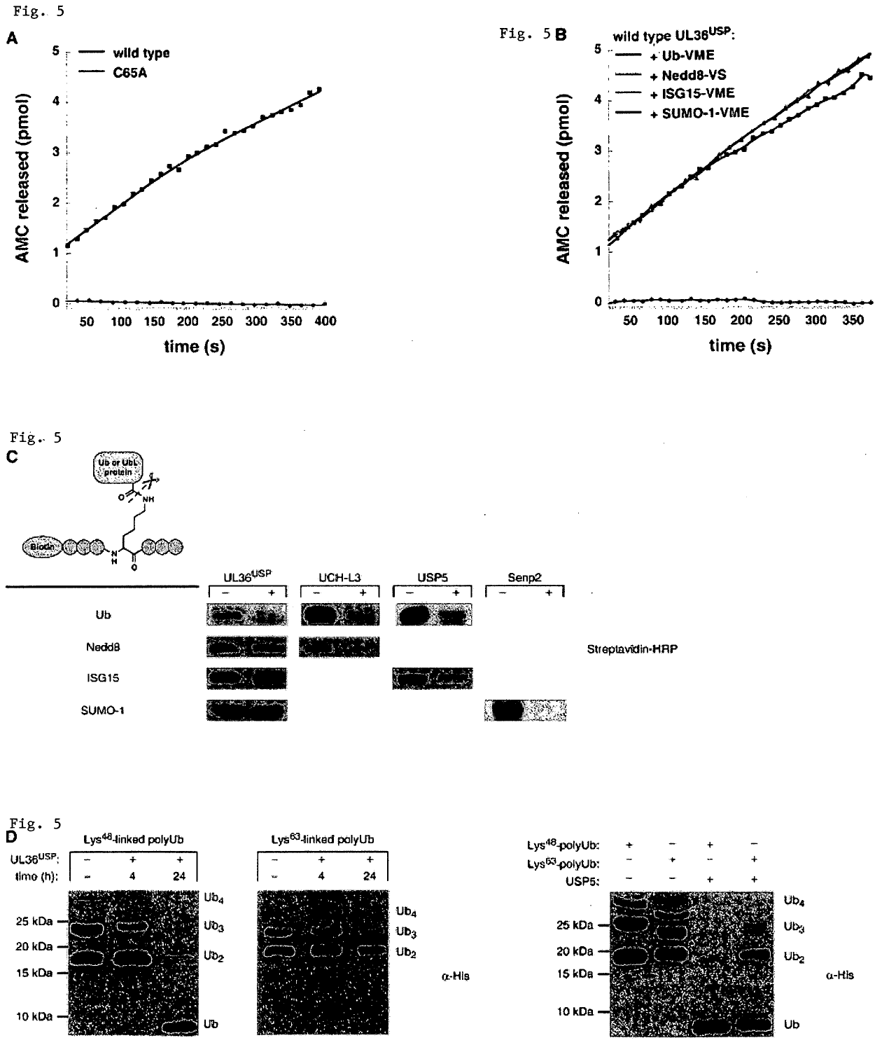

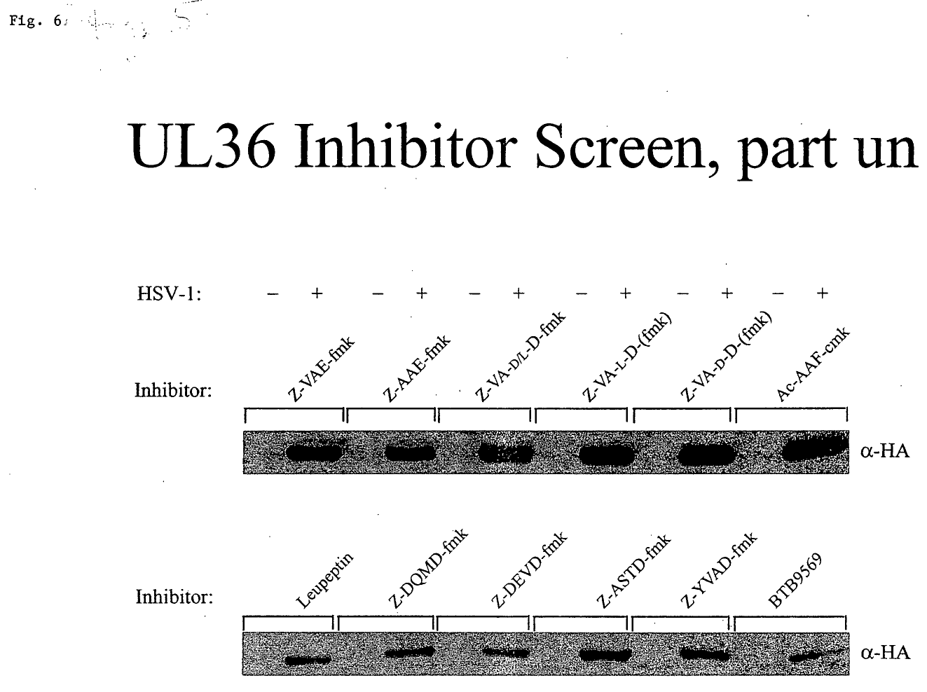

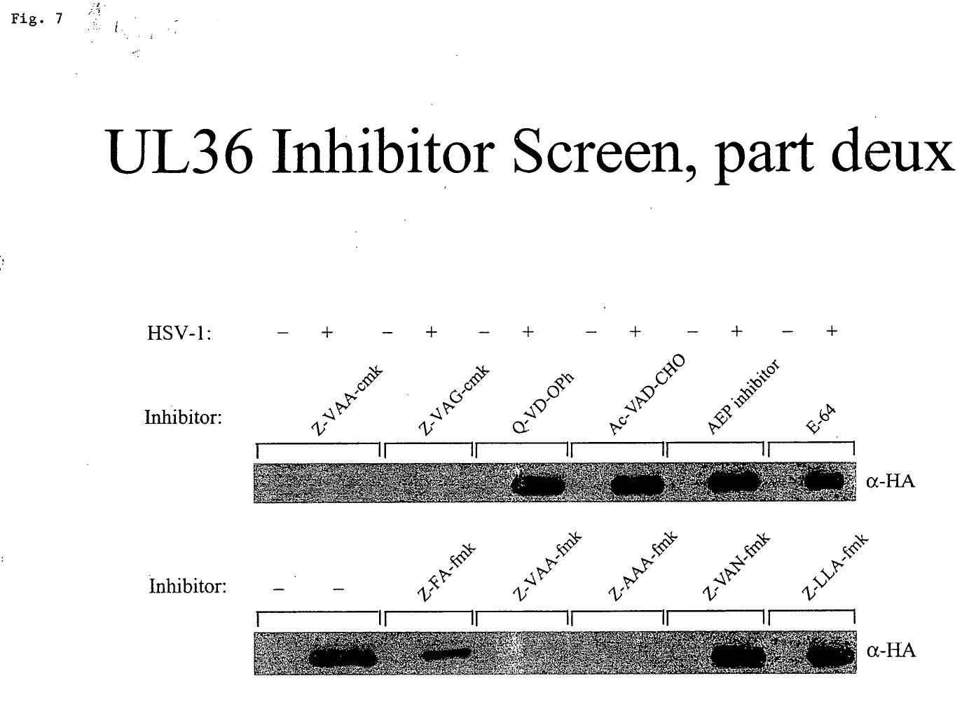

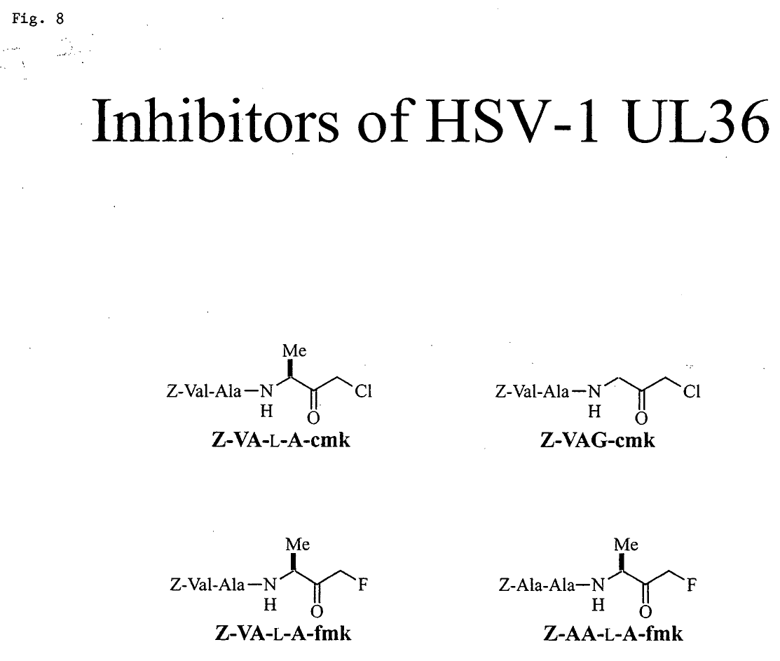

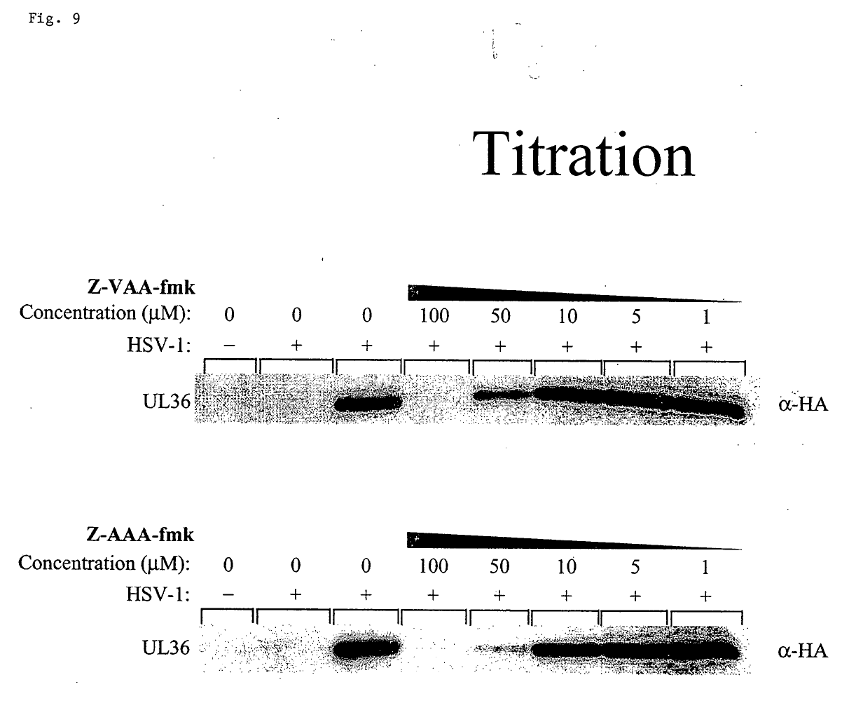

In a typical eukaryotic cell, the presence of multiple DUBs, whose substrate pool and catalytic activities may overlap, confounds straightforward analysis of the activity of any one particular DUB. Although we have found excellent correlation between labeling of polypeptides with the Ub/UbL probes and their identification as active DUBs, the probes are covalent inactivators and cannot be used to assess enzymatic (i.e. catalytic) peptidase activity. To validate the activity of UL36^{USP} as a protease with specificity for Ub-modified substrates, we cloned a sequence spanning the 533 N-terminal amino acids of HSV-1 UL36^{USP} into a His-tagged expression vector (His₆-UL36^{USP}). We also generated a point mutant of this construct in which the active site cysteine residue, Cys⁶⁵, was replaced by alanine (His₆UL36^{USP/C65A}). The proteins were expressed, purified over Ni-NTA resin and evaluated for their ability to cleave Ub C-terminal 7-amido-4-methylcoumarin (Ub-AMC) (Dang et al., 1998). Whereas His₆UL36^{USP} efficiently cleaved Ub-AMC with a turnover number of ˜6 min⁻¹ no cleavage was observed for the C65A mutant, even upon prolonged incubation with the substrate (FIG. 5A). Pre-incubation of His₆UL36^{USP} with UbVME results in complete inhibition of Ub-AMC cleavage by His₆UL36^{USP} (FIG. 5B, red line). In contrast, pre-incubation of His₆UL36^{USP} with SUMO-1-VS, ISG15-VS or Nedd8-VS results in Ub-AMC hydrolysis at rates nearly identical to hydrolysis in the absence of these inhibitors. These data suggest little or no binding of UbLs by UL36^{USP}. The results are consistent with the labeling experiments using UbL probes with HSV-1-infected lysates (FIG. 4B).

Although we observed moderate catalytic efficiency of recombinant UL36^{USP}, this fragment was generated to include residues beyond the estimated processing site of this fragment in vivo. This extension of primary sequence in the recombinant protein may have unrecognized effects on measured UL36^{USP} activity. In addition, the fact that recombinant UL36^{USP} is capable of hydrolyzing Ub-AMC in the absence of additional viral or host cellular proteins does not preclude the possiblity that it may be positively or negatively regulated by other factors or adapters in vivo. Thus, the actual efficiency of UL36^{USP} enzyme during viral replication may differ from that observed for the recombinant fragment.

UL36^{USP} is an Isopeptidase Specific for Ub

Although useful for demonstration of hydrolytic capability and kinetic analysis (Dang et al., 1998), cleavage of Ub-AMC is not directly comparable to cleavage of peptide or isopeptide bonds. To evaluate proteolytic capability, we synthesized substrates of Ub(L) proteins in isopeptide linkage with the lysine residue of a biotinylated 7-mer peptide (FIG. 5C). Incubation of these isopeptide-linked Ub-peptide and UbL-peptide conjugates with HiS₆UL36^{USP} results in cleavage of the Ub-peptide conjugate only (FIG. 5C). Isopeptide-linked conjugates of Nedd8, ISG15 or SUMO-1 are not substrates for UL36^{USP}, again consistent with the data from experiments using the active site-directed UbL probes. No cleavage of Ub/UbL substrates was observed for the active site mutant, His₆UL36^{USP/C65A} (data not shown). These data establish that His₆UL36^{USP} not only reacts with Ub-derived covalent inhibitors such as HAUbVME, but also that this fragment behaves as a protease for the isopeptide-linked ubiquitinated substrates in a manner dependent on the conserved cysteine residue.

UL36^{USP} Cleaves Lys⁴⁸-Linked Polyubiquitin Chains

We further investigated the ability of His₆UL36^{USP} to cleave polyubiquitin chains (polyUb). Chains of Ub linked via Lys⁴⁸ are the canonical signals for targeting ubiquitinated proteins to the proteasome (Chau et al., 1989) although degradation is not the obligatory outcome (Flick et al., 2004). PolyUb linkages are quite diverse, however, as nearly all of the seven lysines of Ub can participate in chain formation (Glickman and Ciechanover, 2002; Kirkpatrick et al., 2005). In addition to Lys⁴⁸-linkages, chains built via Lys⁶³ have been characterized structurally (Tenno et al., 2004; Varadan et al., 2004; Varadan et al., 2002) and functionally. These Lys⁶³-linked polyUb chains serve as signals in DNA damage repair (Ulrich, 2002), inflammatory responses (Sun and Chen, 2004), ribosomal protein synthesis (Spence et al., 2000), and protein trafficking (Hicke and Dunn, 2003). Linkage-selective depolymerization of polyUb in vitro can provide insight into the pool of ubiquitinated substrates on which a given DUB may act. When Lys⁴⁸- and Lys⁶³-linked polyUb was incubated with His₆UL36^{USP}, we observed deubiquitination of Lys⁴⁸-linked polyUb only (FIG. 5D). The positive control, USP5, cleaves both linkages. The failure of UL36^{USP} to cleave Lys⁶³-linked polyUb suggests a possible preference for substrates in which the hydrophobic patch created by Leu⁸, Ile⁴⁴ and Val⁷⁰ of Ub is buried. These residues, important contacts for NZF, UIM, UBA, CUE, and UEV domains (Pickart and Fushman, 2004), are surface-exposed in Lys⁶³-linked chains and buried in Lys⁴⁸-linked polyUb (Tenno et al., 2004; Varadan et al., 2002). Monoubiquitinated substrates or less common polyUb linkages, in which the hydrophobic patch is buried, may also be part of the UL36^{USP} substrate pool.

Discovery of a New Family of Conserved DUBs

The discovery of a new family of conserved deubiquitinating enzymes encoded within the family Herpesviridae indicates that Ub deconjugation plays an important role in the herpesvirus life cycle. The sequences of several examples of DUBs are shown in Table 1. The simplest role for UL36^{USP} is the deubiquitination of other viral proteins in order to prevent their degradation by the proteasome and to increase the amount of protein available for virion assembly. The preference of UL36^{USP} for cleavage of Lys⁴⁸-linked polyubiquitin chains in vitro supports this notion, although we note that activation of UL36^{USP} during infection occurs at a time at which structural protein synthesis and assembly of the capsid have largely been completed.

A null mutation of UL36, containing an internal deletion of 3,600 nucleotides corresponding to amino acids 362 to 1555, has been described. This mutation, ΔUL36, also creates a frame shift and premature stop codon 42 amino acids after the junction of the deletion. The ΔUL36 deletion does not eliminate the active site residues of UL36^{USP}, but it truncates by an estimated ˜110 residues the C-terminus of this fragment and may interfere with proper fragment processing, folding and activity. Although ΔUL36 is a lethal deletion for the virus, large numbers of capsids are produced, which accumulate in the cytoplasm of infected Vero cells. This suggests that supplies of most viral proteins required for capsid assembly are not limiting in the absence of UL36.

A temperature sensitive mutant in UL36, the precise alteration of which was not identified by sequence analysis, also suggests that UL36 is essential (Knipe et al., 1981). At the nonpermissive temperature, the mutation shows defects at a number of stages in the viral life cycle, including release of viral DNA from incoming nucleocapsids, viral DNA synthesis, and late gene expression (Batterson et al., 1983). Previous work also implicates UL36 in sequence-specific interaction with the viral genome (Chou and Roizman, 1989). UL36 recognizes the DR1-U_{c} region of the a sequence of HSV, required for cleavage and packaging of viral DNA. These studies indicate that the UL36 polypeptide is multifunctional, with the exact role of its DUB activity awaiting elucidation.

UL36^{USP} might also play a role in the interactions between virus particles and membrane trafficking components during viral budding. The ubiquitination machinery is part of an existing membrane protein trafficking system involved in the formation of multivesicular bodies (MVBs). This machinery is required for the exit of some viruses, including retroviruses, from infected cells. In mammalian cells, the role of deubiquitination in this process has not been addressed, but a function for deubiquitination during the formation of multivesicular bodies (MVBs) has been described in yeast. Perhaps herpesviruses manipulate ubiquitin modifications during viral egress via the deubiquitinating activity encoded by UL36. This activity may involve the dismantling of Lys⁴⁸-linked polyUb chains to increase the pool of Ub available for conjugation to trafficking virions, or via the direct deubiquitination of mono-ubiquitinated trafficking proteins.

The discovery of a new family of cysteine proteases encoded within the HSV-1 genome suggests that there may be even more roles for controlled deubiquitination—and more DUBs—than previously thought. DUBs are central to many cellular functions, including proteolysis, cell cycle control, receptor internalization, vesicular budding, and sorting within the endo/lysosomal system, but the determinants of substrate recognition and proteolytic specificity among DUBs remain to be fully characterized. This lack of information hinders a thorough understanding of ubiquitination modification as a means of post-translational regulation. Viruses have proven to be useful tools for elucidating similarly complex systems in the past. Studies on viral inhibition of MHC class I trafficking, for example, have greatly increased our understanding of cellular antigen presentation pathways, turnover of N-linked glycoproteins (Misaghi et al., 2004) and ER dislocation mechanisms. Likewise, uncovering the role of UL36^{USP} during herpesvirus infection may uncover new pathways in which DUBs play a role.

Although many DUBs contain conserved motifs that identify them as structurally and, perhaps, functionally related, new classes of DUBs with unique motifs continue to be identified. Proteases with catalytic core motifs similar to those of known specificity can be cross-reactive toward a variety of Ub/UbL proteins, as is the case for UCH-L3 and USP5. Occasionally, these proteases display unanticipated specificity, leading to confusion in nomenclature. Such is the case for Senp8, implicated initially as a SUMO-specific protease based on sequence alignments of the catalytic core domain but, in fact, specific for Nedd8-modified substrates. Likewise, USP18 was suggested to be Ub-specific based on homology to other USPs, but instead appears to be specific for ISG15. This confusion is compounded by the fact that nearly all DUBs have been annotated through the use of bioinformatics approaches—functional data are lacking for the majority of them. Bioinformatic algorithms are not yet capable of accurately predicting specificity/selectivity for Ub or UbL modifiers, leading to discrepancies between nomenclature and functional data. Characterization of any of these proteases, therefore, is not complete until its specificity is assayed, as has been done here, against both Ub and UbL substrates.

The experiments reported here further validate the use of mechanism-based probes for the screening of biological activities. From the observation that UL36^{USP} reacts with a Ub-based electrophilic probe, we inferred that HSV-1 encodes a DUB. This conclusion would have found no support in a bioinformatics-based appraisal, because HSV-1 UL36 does not share homology in sequence or conserved domains with known eukaryotic DUBs or Ub-binding proteins. It is this same lack of homology with cellular counterparts that makes the identification and functional characterization of virus-encoded DUBs so attractive from the perspective of enzymatic inhibitor design. In the absence of similarity between the viral enzyme and cellular counterparts, the likelihood of off-target inhibition by small molecule inhibitors is reduced. The discovery of UL36^{USP} opens up an area of experimentation that should allow functional characterization of components of the Ub pathway in the context of Herpesvirus infection as well as provide new approaches to the treatment and prophylaxis of diseases in both human and non-humans caused by herpesviruses.

### EXAMPLE 2

### A Ubiguitin Deconjugating Activity is Conserved in the Large Tegument Protein of Herpesviridae

The largest tegument protein of human herpes simplex virus, UL36, contains a novel deubiquitinating activity. All members of the herpesviridae contain a homologue of UL36, the N-terminal segment of which shows perfect conservation of those residues implicated in catalysis in the UL36 of HSV-1. For cytomegalovirus and epstein-barr virus, chosen as representatives of the beta- and gamma-herpesvirus subfamilies, we show that the homologous modules display deubiquitination activity in vitro. This enzyme and its deubiquitinating activity are conserved throughout all subfamilies of Herpesviridae.

Example 1 describes the identification of a novel viral ubiquitin specific protease (USP), UL36USP, encoded by the herpes simplex virus-1 (HSV-1) genome. UL36USP is polypeptide of approximately 420 amino acids (AA) encoded within the N-terminal portion of UL36, the largest tegument protein (3164 AA) of HSV-1. This activity was detected through the use of mechanism based, active site directed probes, and confirmed by expression in Escherichia coli of a corresponding fragment that cleaves ubiquitin-based substrates. UL36USP activity peaks at late stages of viral replication and appears to require the proteolytic processing from full length UL36. Although the N-terminal UL36 fragment is well conserved in α-herpesviruses, a low but significant homology to corresponding genes of the β and γ subfamilies was apparent in sequence alignments, with strict conservation of the proposed catalytic residues.

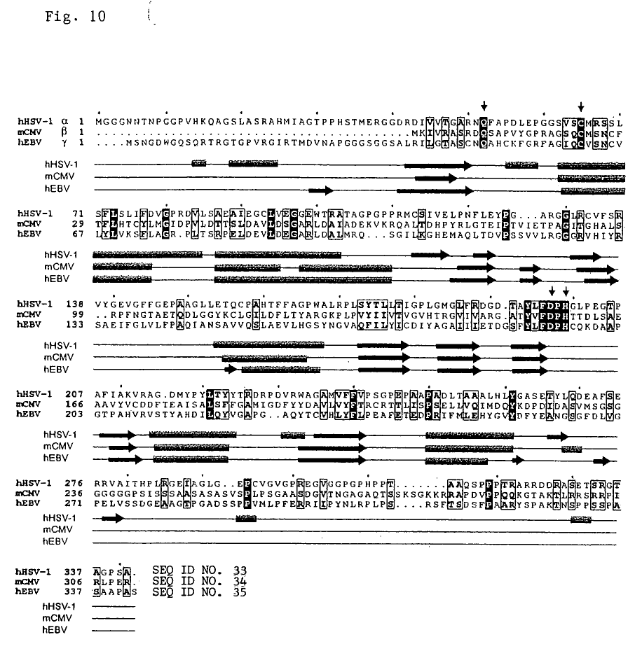

We therefore set out to investigate the possible DUB activity of two phylogenetically distant homologues of HSV-1 UL36USP, each representing a different subfamily of herpesviridae. We chose UL36 homologues encoded by mouse cytomegalovirus (mCMV, UL48) and Epstein-barr virus (EBV, BPLF1) as representatives of the β and γ-subfamilies, respectively. In order to assess the degree of homology between UL36 from HSV-1 and its mCMV and EBV counterparts, a sequence alignment was generated covering the first 336 AA (numbering refers to HSV-1) of UL36 (FIG. 10). Overall, the homology to HSV-1 is rather low, with only 10% and 15% sequence identity for mCMV and EBV, respectively. Nevertheless, the putative catalytic triad Cys-His-Asp is strictly conserved, along with a putative oxyanion hole-forming Gln residue (FIG. 10). The conserved Cys65 is the active site cysteine in HSV-1 UL36USP. We therefore propose that the two homologues under investigation likewise represent a cysteine protease-type DUB. In support of this notion, a secondary structure prediction shows a high degree of structural similarity despite limited sequence identity, suggesting that all members might adopt a similar tertiary structure (FIG. 10). Based on the observations that (i) the putative catalytic triad is contained within a stretch of ˜200 AA and (ii) the secondary structure prediction diverges beyond position ˜280, we reasoned that a fragment of 280 AA or less should include the minimal domain necessary and sufficient for catalysis.

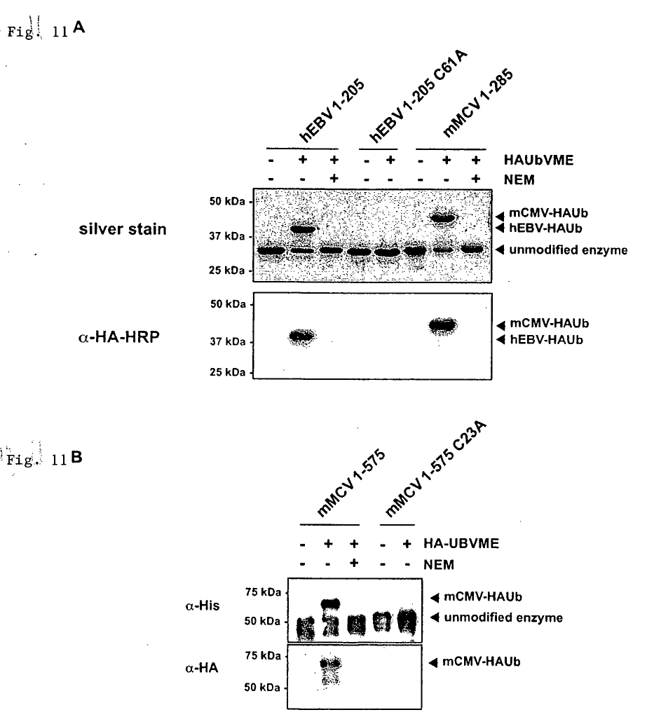

To investigate functionality of this minimal DUB consensus domain we cloned the relevant genomic fragments for heterologous expression and subsequent biochemical characterization. The corresponding DNA fragments, encoding for AA 1-205 of EBV (EBV205) and AA 1-285 of mCMV (mCMV285), were PCR-cloned into pET28 according to standard procedures. Following expression in E. coli BL21-DE3, the fragments bearing a N-terminal His-tag were purified using a Ni-NTA resin (Qiagen) and subsequently subjected to a gel filtration column (S75 HiLoad, Amersham Pharmacia Biotech) equilibrated in storage buffer (50 mM Tris, 50 mM KCl, 50 mM NaCl, 0.5 mM EDTA, 2 mM DTT, 10% (v/v) glycerol, pH 7.5) to achieve apparent homogeneity. To address whether the purified constructs display DUB activity, we used HA-tagged Ub-vinylmethyl ester (HAUbVME), a probe that acts as suicide substrate for DUBs by forming a thioether bond to the active site cysteine. Reactions were performed using final concentrations of 1 μM enzyme (EBV205 or mCMV285) in the absence and presence of 2 μM HAUbVME in reaction buffer (50 mM Tris, 100 mM NaCl, 1 mM DTT pH 7.5) for 30 min at 37° C. Following incubation, samples were boiled in reducing sample buffer and subjected to SDS-PAGE. A shift in electrophoretic mobility, indicative of a covalent modification by HAUb, was observed for both the mCMV and EBV constructs after silver staining (FIG. 11 A, upper panel). The identity of the covalent enzyme-HAUb adducts was confirmed by immunoblotting (FIG. 11 A, lower panel), using a HRP-conjugated anti-HA antibody (3F10, Roche, 1:4000 dilution) in conjunction with the Western Lightning chemiluminescence reagent kit (Perkin Elmer).

Pretreatment of EBV205 or mCMV285 with 10 mM N-ethylmaleimide (NEM) for 10 min completely blocked labeling with the probe (FIG. 11 A). Replacement of the putative active site cysteine by alanine (C61A) abrogated labeling with the probe for EBV205 (FIG. 11 A). In the case of mCMV, we also constructed a longer variant that terminates at position 575 (mCMV575). A polypeptide of the expected molecular mass (62 kDa) was clearly detectable by immuno blotting using a penta-His™Qiagen) antibody (FIG. 11 B). This variant likewise reacted with HAUbVME in NEM-sensitive fashion, and a non-conservative mutation of the putative active site (C23A) resulted in complete loss of labeling with the probe (FIG. 11 B). Taken together with the established specificity of the electrophilic derivatives of Ub, we conclude that both the mCMV and EBV-derived fragments are Ub specific cysteine proteases.

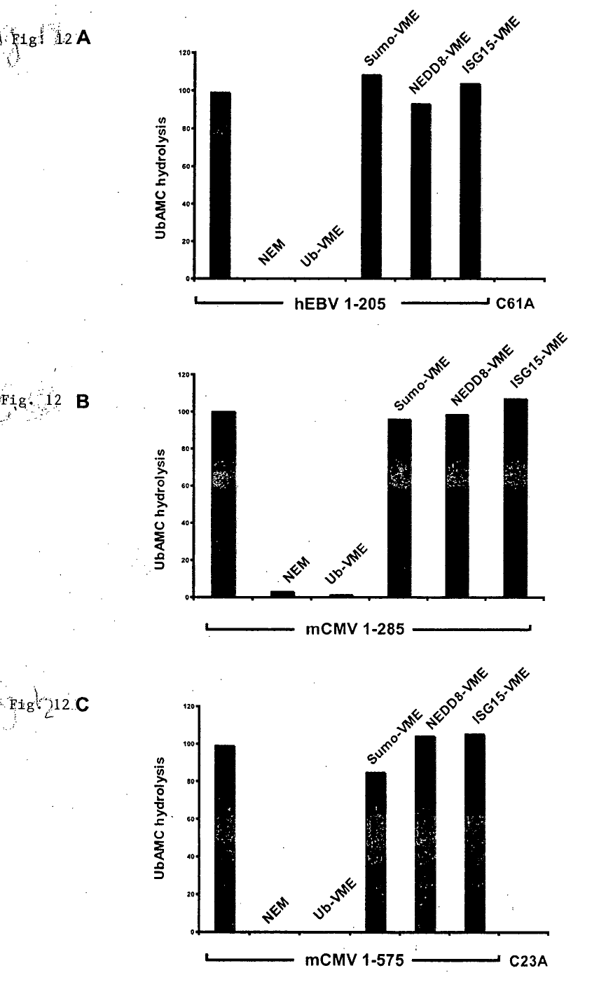

We next tested the ability of the enzymes to cleave Ub-AMC (Ub C-terminal 7-amido-4-methylcoumarin, Boston Biochem). UbAMC hydrolysis assays were performed in reaction buffer supplemented with BSA (50 μg/ml) by incubating the enzymes (100 pM) with a large excess of Ub-AMC (100 nM). EBV205, mCMV285 and mCMV575 efficiently hydrolyzed UbAMC, as had been shown for UL36USP. In accordance with HA-UbVME labeling experiments, UbAMC hydrolysis was sensitive to NEM and absent in the case of active site mutants, corroborating the identity of the proposed catalytic cysteine residue (FIGS. 12 A-C). While a minimal DUB domain extending little beyond the active site residues is active, we sought to determine whether the shorter constructs are sufficient also to confer substrate specificity. To address this issue, we tested the potential of several inhibitors to block DUB activity in Ub-AMC hydrolysis assays. Enzymes were preincubated for 1 h with a 100-fold molar excess of electrophilic derivatives of Ub, SUMO, NEDD8, and ISG15, all carrying a VME moiety as the electrophile at their C-terminus. While UbVME completely inhibited hydrolytic activity, none of the Ub-related inhibitors impaired DUB activity (FIG. 3). We conclude that a domain consisting of little more than 200 AA, as exemplified by EBV205, mediates both catalytic activity and specificity to Ub.

The data described herein indicate that the DUB activity previously identified in HSV-1 is conserved across all subfamilies of herpesviridae. Unexpectedly, a comparatively small conserved module of ˜200 AA is sufficient for both hydrolytic activity and Ub-specificity. Sequence elements adjacent to the 200 AA core domain may be required to confer additional specificity for ubiquitinylated substrates in vivo or may be required to mediate other interactions required for e.g. subcellular localization. Having demonstrated the enzymatic activity embedded within the tegument proteins of the α, β and γ herpesviruses, we postulate an important and conserved function for this activity, the identification and further analysis of which will may be facilitated by the creation of suitable mutants in the context of the intact genomes of the corresponding viruses.

### EXAMPLE 3

### Construction and Testing of BAC Strains Carrying Mutations in the USP Region of the MCMV M48 Gene

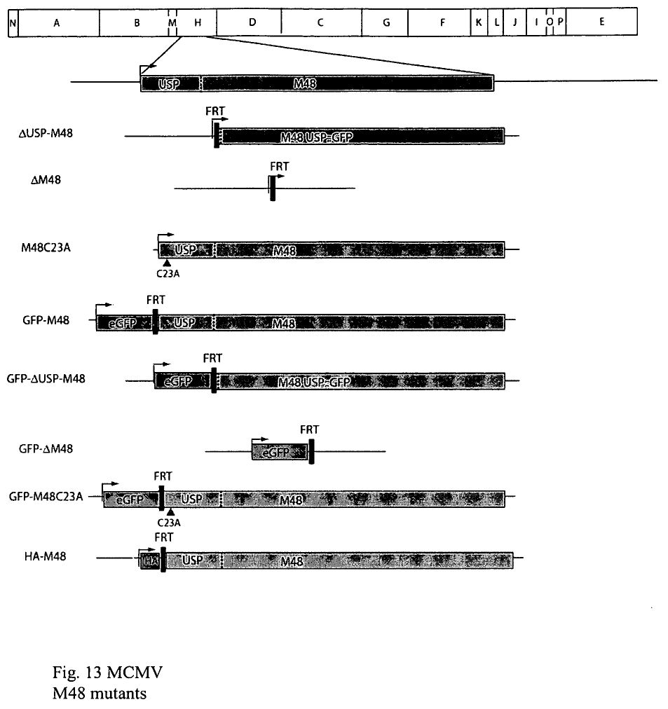

We constructed mutations in the MCMV M48 gene, which is associated with DUB activity, using FRT-mediated mutagenesis and “en passant” mutagenesis (Wagner et al., Trends Microbiol. 10:318-24,2002; Tischer et al., Biotechniques 40:191-97, 2006). The M48 mutants are shown in FIG. 13. All mutants were reconstituted and are viable in vivo except for the ΔM48 and GFP-ΔM8. The lethality of the full deletion mutants is consistent with the literature suggesting M48 and its homologs are essential genes.

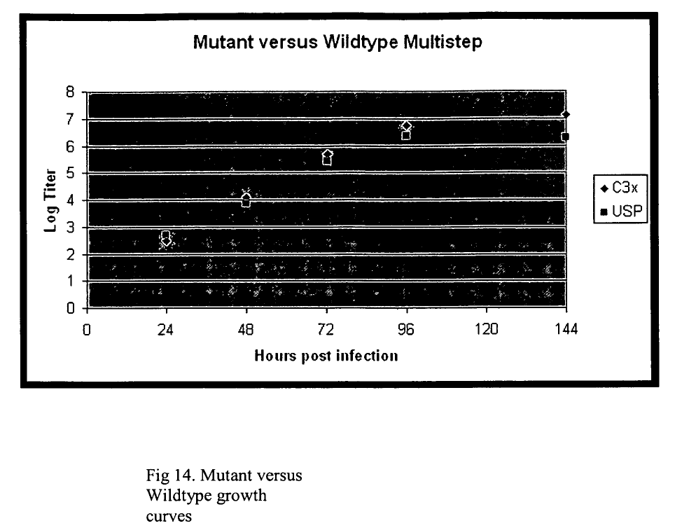

We characterized the mutant phenotypes in vitro and in vivo. When assayed in vitro, mutations in the USP region of M48 did not appear to significantly compromise growth at low or high MOIs in cell culture. An example of a multistep growth curve carried out at an MOI of 1 is seen in FIG. 14.

One experiment was performed characterizing mutant growth in vivo. The results suggest that the ΔUSP-M48 mutant is replication competent, but unable to establish an efficient infection of the host. Titers from Day 7 organs are shown in FIG. 14. These results indicate that that the herpesvirus proteases are useful drug targets in fighting infection in vivo. It appears that the herpesvirus proteases, although not essential in tissue culture, may be performing an essential role in establishment of infection in the host.

### EXAMPLE 4

### Targets of M48 Deubiquitinating Activity

We examined the cellular targets of the M48 deubiquitinating activity. HA-Ub MEFs were lysed in NP40 lysis buffer in the absence of protease inhibitors. Recombinantly expressed M48^{USP} or an active site Cysteine/Alanine M48 mutant was added at the indicated concentrations. Lysates were then run on an SDS-Page gel and immunoblotted with anti-HA (12CA5) antibody.

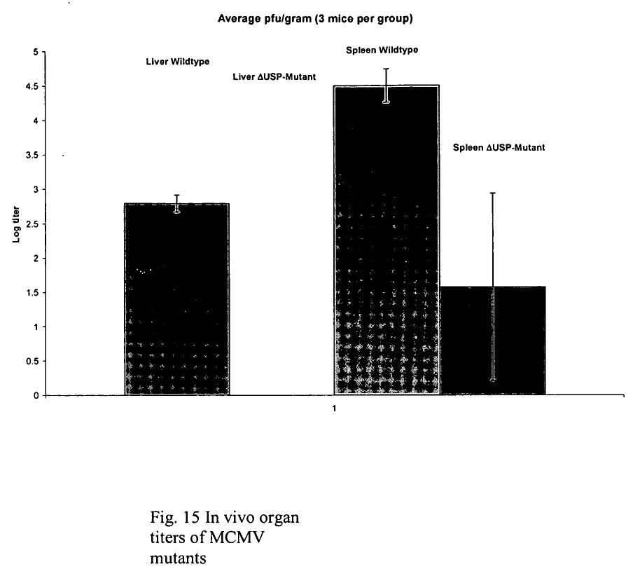

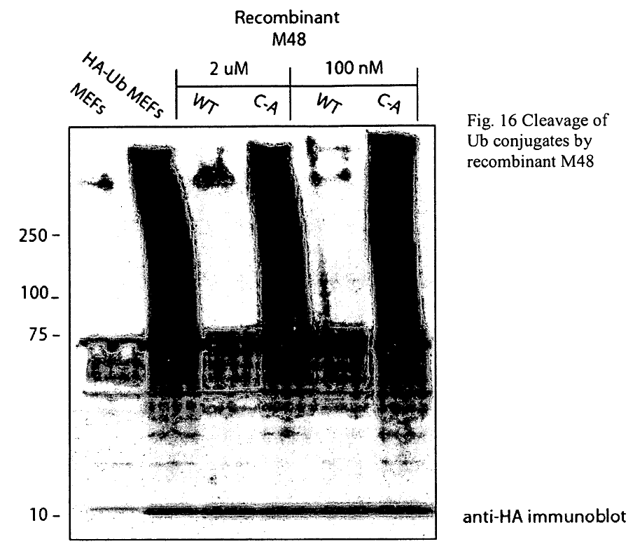

The results are shown in FIG. 15. M48 is not specific when added recombinantly to cell lysate. It is clear that recombinant M48 can cleave Ub from the majority of proteins within a cell lysate. This results demonstrates that an HSV-derived DUB protease can be use as an in vitro tool in assays requiring universal ubiquitin removal, for example, in removing ubiquitin from beads during an immunoprecipitation. The universal specificity of these proteases for all ubiquitin conjugates suggests they are useful in vitro tools for manipulating ubiquitinated proteins in vitro.

Those skilled in the art will recognize, or be able to ascertain using no more than routine experimentation, many equivalents to the specific embodiments of the invention described herein. The scope of the present invention is therefore not intended to be limited to the above Description.

What is claimed is:

1. A ubiquitin specific protease comprising a Cys box, said Cys box being selected from the group consisting of SEQ ID NO:1 or SEQ ID NO:2 and a His box being selected from SEQ ID NO:3 and SEQ ID NO:4, wherein said protease is encoded by a naturally-occurring Herpes virus gene sequence.

2. The protease of claim 1, wherein said protease is not encoded by a naturally-occurring eucaryotic cell gene sequence

3. The protease of claim 1, wherein said the location of said Cys box is N-terminal to the location of said His box.

4. The protease of claim 1, wherein said Cys box and said His box are separated by 75 to 90 amino acids.

5. The protease of claim 1, wherein said Cys box and said His box are separated by 75-85 amino acids.

6. The protease of claim 1, wherein said Cys box and said His box are separated by 78, 79, 80, 81, 82, 83, 84, or 85 amino acids.

7. The protease of claim 1, wherein said Cys box is selected from the group consisting of SEQ ID NO:5, 6, 7, 8, 9, 10, 11, and 12.

8. The protease of claim 1, wherein said His box is selected from the group consisting of SEQ ID NO: 13, 14, 15, 16, 17, 18, 19, and 20.

9. An inhibitor of a deubiquitinating enzyme, wherein said inhibitor preferentially inhibits a Herpes virus enzyme compared to a cellular enzyme.

10. The inhibitor of claim 7, wherein said inhibitor is a methyl ketone compound.

11. The inhibitor of claim 7, wherein said inhibitor is an alpha-halogenated methyl ketone compound.

12. The inhibitor of claim 7, wherein said inhibitor is selected from the group consisting of Z-Z-VA-L-A-cmk, Z-VA-L-A-fmk, Z-VAG-cmk, and Z-AA-L-A-fmk.

13. A method of inhibiting Herpes virus replication, comprising contacting a virally-infected cell with the inhibitor of claim 9.

14. A method of inhibiting a Herpes virus infection in a subject, comprising administering to said subject the inhibitor of claim 9.

15. A method of deubiquitinating a ubiquitinated protein, the method comprising
providing a ubiquitinated protein and an HSV DUB protease; and
contacting said ubiquitinated protein and HSV DUB protease under conditions sufficient to remove ubiquitin from said protein.

16. The method of claim 15, wherein said ubiquitinated protein is provided on a bead.

17. The method of claim 15, wherein said HSV DUB protease is a fragment of a UL36 HSV-1 protein, or counterpart of a UL36 HSV-1 protein from another herpesviridae member, and wherein said fragment has ubiquitin specific protease activity.

18. A kit for deubiquitinating a ubiquitinated protein comprising an effective amount of a preparation of an HSB DUB protease and a container.


SEQUENCE LISTING (56 sequences)

SEQ ID NO: 1 - protein, 47 aa, Homo sapiens
  MISC_FEATURE (1)..(1): x is N or H
  MISC_FEATURE (3)..(7): x is any amino acid
  MISC_FEATURE (8)..(8): x is e or g
  MISC_FEATURE (9)..(11): x is any amino acid
  MISC_FEATURE (12)..(12): x is s or g
  MISC_FEATURE (13)..(14): x is any amino acid
  MISC_FEATURE (16)..(16): x is m, l or v
  MISC_FEATURE (17)..(21): x is any amino acid
  MISC_FEATURE (22)..(22): x is f or y
  MISC_FEATURE (24)..(26): x is any amino acid
  MISC_FEATURE (27)..(27): x is f, y or h
  MISC_FEATURE (28)..(29): x is any amino acid
  MISC_FEATURE (30)..(30): x is g or s
  MISC_FEATURE (31)..(34): x is any amino acid
  MISC_FEATURE (35)..(35): x is l, t or v
  MISC_FEATURE (36)..(40): x is any amino acid
  MISC_FEATURE (41)..(41): x is e or d
  MISC_FEATURE (42)..(43): x is any amino acid
  MISC_FEATURE (44)..(44): x is l or m
  MISC_FEATURE (45)..(45): x is v, q, d or e
  MISC_FEATURE (46)..(46): x is e, n or q
XQXXXXXXXXXXXXCXXXXXXXLXXXXXXXXXXXXXXXXXXXXXXXG

SEQ ID NO: 2 - protein, 48 aa, Homo sapiens
  MISC_FEATURE (1)..(1): x is n or h
  MISC_FEATURE (3)..(8): x is any amino acid
  MISC_FEATURE (9)..(9): x is e or g
  MISC_FEATURE (10)..(12): x is any amino acid
  MISC_FEATURE (13)..(13): x is s or g
  MISC_FEATURE (13)..(23): x is f or y
  MISC_FEATURE (14)..(15): x is any amino acid
  MISC_FEATURE (17)..(17): x is m, l or v
  MISC_FEATURE (18)..(22): x is any amino acid
  MISC_FEATURE (25)..(27): x is any amino acid
  MISC_FEATURE (28)..(28): x is f, y or h
  MISC_FEATURE (29)..(30): x is any amino acid
  MISC_FEATURE (31)..(31): x is g or s
  MISC_FEATURE (32)..(35): x is any amino acid
  MISC_FEATURE (36)..(36): x is l, t or v
  MISC_FEATURE (37)..(41): x is any amino acid
  MISC_FEATURE (42)..(42): x is e or d
  MISC_FEATURE (43)..(44): x is any amino acid
  MISC_FEATURE (45)..(45): x is l or m
  MISC_FEATURE (46)..(46): x is v, q, d or e
  MISC_FEATURE (47)..(47): x is e, n or q
XQXXXXXXXXXXXXXCXXXXXXXLXXXXXXXXXXXXXXXXXXXXXXXG

SEQ ID NO: 3 - protein, 30 aa, Homo sapiens
  MISC_FEATURE (1)..(1): x is t or c
  MISC_FEATURE (2)..(2): x is i, v, d or q
  MISC_FEATURE (3)..(13): x is any amino acid
  MISC_FEATURE (14)..(14): x is d, n, g, e or k
  MISC_FEATURE (15)..(16): x is any amino acid
  MISC_FEATURE (17)..(17): x is y, f or l
  MISC_FEATURE (18)..(18): x is l, i, v or m
  MISC_FEATURE (21)..(21): x is p or s
  MISC_FEATURE (23)..(29): x is any amino acid
  MISC_FEATURE (30)..(30): x is a, g or s
XXXXXXXXXXXXXXXXXXFDXHXXXXXXXX

SEQ ID NO: 4 - protein, 29 aa, Homo sapiens
  MISC_FEATURE (1)..(1): x is t or c
  MISC_FEATURE (2)..(2): x is i, v, d or q
  MISC_FEATURE (3)..(12): x is any amino acid
  MISC_FEATURE (13)..(13): x is d, n, g, e or k
  MISC_FEATURE (14)..(15): x is any amino acid
  MISC_FEATURE (16)..(16): x is y, f or l
  MISC_FEATURE (17)..(17): x is l, i, v or m
  MISC_FEATURE (20)..(20): x is p or s
  MISC_FEATURE (22)..(28): x is any amino acid
  MISC_FEATURE (29)..(29): x is a, g or s
XXXXXXXXXXXXXXXXXFDXHXXXXXXXX

SEQ ID NO: 5 - protein, 47 aa, human herpesvirus 1
NQFAPDLEPGGSVSCMRSSLSFLSLIFDVGPRDVLSAEAIEGCLVEG

SEQ ID NO: 6 - protein, 47 aa, human herpesvirus 2
NQFATDLEPGGSVSCMRSSLSFLSLLFDVGPRDVLSAEAIEGCLVEG

SEQ ID NO: 7 - protein, 47 aa, Human herpesvirus 3
NQFDGALGPASGLSCLRTSLSFLHMTYAHGINATLSSDMIDGCLQEG

SEQ ID NO: 8 - protein, 46 aa, Human herpesvirus 4
NQAHCKFGRFAGIQCVSNCVLYLVKSFLAGRPLTSRPELDEVLDEG

SEQ ID NO: 9 - protein, 48 aa, Human herpesvirus 5
HQGDIARFGARAGNQCVCNGIMFLHALHLGGTSAVLQTEALDAIMEEG

SEQ ID NO: 10 - protein, 47 aa, Human herpesvirus 6
NQNDSKYGPRAGKQCMSNSFSFLHTVYLNGINNSLNAGTIDAIMEEG

SEQ ID NO: 11 - protein, 47 aa, Human herpesvirus 7
NQNDPKYGPRAGKQCMSNCFSFLHTVYLNGINNVLNKESIDIIMENG

SEQ ID NO: 12 - protein, 47 aa, Human herpesvirus 8
HQANCIFGEHAGSQCLSNCVMYLASSYYNSETPLVDRASLDDVLEQG

SEQ ID NO: 13 - protein, 30 aa, human herpesvirus 1
TIGPLGMGLFRDGDTAYLFDPHGLPEGTPA

SEQ ID NO: 14 - protein, 30 aa, human herpesvirus 2
TIGPLGMGLYRDGDTAYLFDPHGLPAGTPA

SEQ ID NO: 15 - protein, 30 aa, Human herpesvirus 4
TVDSTGMAIYRQGNISFIFDPHGHGTIGQA

SEQ ID NO: 16 - protein, 30 aa, Human herpesvirus 5
CDIYAGAIIIETDGSFYLFDPHCQKDAAPG

SEQ ID NO: 17 - protein, 30 aa, Human herpesvirus 5
TVNSLARAVVFTEDHMLVFDPHSSAECHNA

SEQ ID NO: 18 - protein, 30 aa, Human herpesvirus 6
TIGVLSRALFFLNNTLYLFDSHPTEREATA

SEQ ID NO: 19 - protein, 30 aa, Human herpesvirus 7
TIGVMARAIFIVDELFYLFDSHASDTENSA

SEQ ID NO: 20 - protein, 29 aa, Human herpesvirus 8
CQSKAGAIVVKDKTYYNFDPHCIPNIPNS

SEQ ID NO: 21 - protein, 23 aa, Homo sapiens
NQFAPDLEPGGSVSCMRSSLSFL

SEQ ID NO: 22 - protein, 26 aa, Homo sapiens
VYFMKQTIGNSCGTIGLIHAVANNQD

SEQ ID NO: 23 - protein, 21 aa, Homo sapiens
VGLKNQGATCYMNSLLQTLFF

SEQ ID NO: 24 - protein, 24 aa, Homo sapiens
LLPLATTGDGNCLLHAASLGMWGF

SEQ ID NO: 25 - protein, 24 aa, Homo sapiens
IMIGKKKGIQGHYNSCYLDSTLFC

SEQ ID NO: 26 - protein, 25 aa, Homo sapiens
ESIFHEKQEGSLCAQHCLNNLLQGE

SEQ ID NO: 27 - protein, 22 aa, Homo sapiens
FDPHGLPEGTPAFIAKVRAGDM

SEQ ID NO: 28 - protein, 29 aa, Homo sapiens
FHFILFNNVDGHLYELDGRMPFPVNHGAS

SEQ ID NO: 29 - protein, 41 aa, Homo sapiens
PANYILHAVLVHSGDNHGGHYVVYLNPKGDGKWCKFDDDVV

SEQ ID NO: 30 - protein, 16 aa, Homo sapiens
SPLVLAYDQAHFSALV

SEQ ID NO: 31 - protein, 21 aa, Homo sapiens
ETSHYVAFVKYGKDDSAWLFF

SEQ ID NO: 32 - protein, 21 aa, Homo sapiens
NERSFICNYKEHWFTVRKLGK

SEQ ID NO: 33 - protein, 340 aa, herpes simplex virus 1
MGGGNNTNPGGPVHKQAGSLASRAHMIAGTPPHSTMERGGDRIVVTGARNQFAPDLEPGGSVSCMRSSLSFLSLIFDVGPRDVLSAEAIEGCLVEGGEWTRATAGPGPPRMCSIVELPNFLEYPGARGGLRCVFSRVYGEVGFFGEPAAGLLETQCPAHTFFAGPWALRPLSYTLLTIGPLGMGLFRDGDTAYLFDPHGLPEGTPAFIAKVRAGDMYPYLTYYTRDRPDVRWAGAMVFFVPSGPEPAAPADLTAAALHLYGASETYLQDEAFSERRVAITHPLRGEIAGLGEPCVGVGPREGVGGPGPHPPTAAQSPPPTRARRDDRASETSRGTAGPSA

SEQ ID NO: 34 - protein, 311 aa, Mouse cytomegalovirus 1
MKIVRASRDQSAPVYGPRAGSQCMSNCFTFLHTCYLMGIDPVLDTTSLDAVLDSGARLDAIADEKVKRQALTDHPYRLGTEIPTVIETPAGITGHALSRPFNGTAETQDLGGYKCLGILDFLTYARGKPLPVYIIVTVGVHTRGVIVARGATYVFDPHTTDLSAEAAVYVCDDFTEAISALSFFGAMIGDFYYDAVLVYFTRCRTTLISPEELLVQIMDQYKDPDIDASVMSGSGGGGGGPSISSSAASASASVSPLPSGAASDGVTNGAGAQTSSKSGKKRRAPDVPPQQKGTTAKTLRRSRRPIRLPER

SEQ ID NO: 35 - protein, 342 aa, Epstein-barr virus
MSNGDWGQSQRTRGTGPVRGIRTMDVNAPGGGSGGSALRILGTASCNQAHCKFGRFAGIQCVSNCVLYLVKSFLAGRPLTSRPELDEVLDEGARLDALMRQSGILKGHEMAQLTDVPSSVVLRGGGRVHIYRSAEIFGLVLEPAQIANSAVVQSLAEVLHGSYNGVAQFILYICDIYAGAIIIETDGSFYLFDPHCQKDAAPGTPAHVRVSTYAHDILQYVGAPGAQYTCVHLYFLPEAFETEDPRIFMLEHYGVYDFYEANGSGFDLVGPELVSSDGEAAGTPGADSSPPVMLPFERRIIPYNLRPLPSRSFTSDSFPAARYSPAKTNSPPSSPASAAPAS

SEQ ID NO: 36 - protein, 3164 aa, human herpesvirus 1
MGGGNNTNPGGPVHKQAGSLASRAHMIAGTPPHSTMERGGDRDIVVTGARNQFAPDLEPGGSVSCMRSSLSFLSLIFDVGPRDVLSAEAIEGCLVEGGEWTRATAGPGPPRMCSIVELPNFLEYPGARGGLRCVFSRVYGEVGFFGEPAAGLLETQCPAHTFFAGPWALRPLSYTLLTIGPLGMGLFRDGDTAYLFDPHGLPEGTPAFIAKVRAGDMYPYLTYYTRDRPDVRWAGAMVFFVPSGPEPAAPADLTAAALHLYGASETYLQDEAFSERRVAITHPLRGEIAGLGEPCVGVGPREGVGGPGPHPPTAAQSPPPTRARRDDRASETSRGTAGPSAKPEAKRPNRAPDDVWAVALKGTPPTDPPSADPPSADPPSAIPPPPPSAPKTPAAEAAEEDDDDMRVLEMGVVPVGRHRARYSAGLPKRRRPTWTPPSSVEDLTSGEKTKRSAPPAKTKKKSTPKGKTPVGAAVPASVPEPVLASAPPDPAGPPVAEAGEDDGPTVPASSQALEALKTRRSPEPPGADLAQLFEAHPNVAATAVKFTACSAALAREVAACSRLTISALRSPYPASPGLLELCVIFFFERVLAFLIENGARTHTQAGVAGPAAALLEFTLNMLPWKTAVGDFLASTRLSLADVAAHLPLVQHVLDENSLIGRLALAKLILVARDVIRETDAFYGELADLELQLRAAPPANLYTRLGEWLLERSQAHPDTLFAPATPTHPEPLLYRVQALAKFARGEEIRVEAEDRQMREALDALARGVDAVSQHAGPLGVMPAPAGAAPQGAPRPPPLGPEAVQVRLEEVRTQARRAIEGAVKEYFYRGAVYSAKALQASDNNDRRFHVASAAVVPVVQLLESLPVFDQHTRDIAQRAAIPAPPPIATSPTAILLRDLIQRGQTLDAPEDLAAWLSVLTDAANQGLIERKPLDELARSIRDINDQQARRSSGLAELRRFDALDAALGQQLDSDAAFVPAPGASPYPDDGGLSPEATRMAEEALRQARAMDAAKLTAELAPDARARLRERARSLEAMLEGARERAKVARDAREKFLHKLQGVLRPLPDFVGLKACPAVLATLRASLPAGWSDLPEAVRGAPPEVTAALRADMWGLLGQYRDALEHPTPDTATALSGLHPSFVVVLKNLFADAPETPFLLQFFADHAPIIAHAVSNAINAGSAAVATADPASTVDAAVRAHRVLVDAVTALGAAASDPASPLAFLAAMADSAAGYVKATRLALDARVAIAQLTTLGSAAADLVVQVRRAANQPEGEHASLIQAATRATTGARESLAGHEGRFGGLLHAEGTAGDHSPSGRALQELGKVIGATRRRADELEAATADLREKMAAQRARSSHERWAADVEAVLDRVESGAEFDVVELRRLQALAGTHGYNPRDFRKRAEQALGTNAKAVTLALETALAFNPYTPENQRHPMLPPLAAIHRIDWSAAFGAAADTYADMFRVDTEPLARLLRLAGGLLERAQANDGFIDYHEAVLHLSEDLGGVPALRQYVPFFQKGYAEYVDIRDRLDALRADARRAIGSVALDLAAAAEEISAVRNDPAAAAELVRAGVTLPCPSEDALVACVAALERVDQSPVKDTAYAHYVAFVTRQDLADTKDAVVRAKQQRAEATERVTAGLREVLAARERRAQLEAEGLANLKTLLKVVAVPATVAKTLDQARSAEEIADQVEILVDQTEKARELDVQAVAWLEHAQRTFETHPLSAASGDGPGLLTRQGARLQALFDTRRRVEALRRSLEEAEAEWDEVWGRFGRVRGGAWKSPEGFRAACEQLRALQDTTNTVSGLRAQRDYERLPAKYQGVLGAKSAERAGAVEELGGRVAQHADLSARLRDEVVPRVAWEMNFDTLGGLLAEFDAVAGDLAPWAVEEFRGARELIQRRMGLYSAYAKATGQTGAGAAAAPAPLLVDLRALDARARASAPPGQEADPQMLRRRGEAYLRVSGGPGPLVLREATSTLDRPFAPSFLVPDGTPLQYALCFPAVTDKLGALLMCPEAACIRPPLPTDTLESASTVTAMYVLTVINRLQLALSDAQAANFQLFGRFVRHRQARWGASMDAAAELYVALVATTLTREFGCRWAQLEWGGDAAAPGPPLGPQSSTRHRVSFNENDVLVALVASSPEHIYTFWRLDLVRQHEYMHLTLPRAFQNAADSMLFVQRLTPHPDARIRVLPAFSAGGPPTRGLMFGTRLADWRRGKLSETDPLAPWRSVPELGTERGAALGKLSPAQALAAVSVLGRMCLPSTALVALWTCMFPDDYTEYDSFDALLTARLESGQTLSPSGGREASPPAPPNALYRPTGQHVAVPAAATHRTPAARVTAMDLVLAAVLLGAPVVVALRNTTAFSRESELELCLTLFDSRARGPDAALRDAVSSDIETWAVRLLHADLNPIENACLAAQLPRLSALIAERPLARGPPCLVLVDISMTPVAVLWENPDPPGPPDVRFVGSEATEELPFVAGGEDVLAASATDEDPFLARAILGRPFDASLLSGELFPGHPVYQRAPDDQSPSVPNPTPGPVDLVGAEGSLGPGSLAPTLFTDATPGEPVPPRMWAWIHGLEELASDDSGGPAPLLAPDPLSPTADQSVPTSQCAPRPPGPAVTAREARPGVPAESTRPAPVGPRDDFRRLPSPQSSPAPPDATAPRPPASSRASAASSSGSRARRHRRARSLARATQASATTQGWRPPALPDTVAPVTDFARPPAPPKPPEPAPHALVSGVPLPLGPQAAGQASPALPIDPVPPPVATGTVLPGGENRRPPLTSGPAPTPPRVPVGGPQRRLTRPAVASLSESRESLPSPWDPADPTAPVLGRNPAEPTSSSPAGPSPPPPAVQPVAPPPTSGPPPTYLTLEGGVAPGGPVSRRPTTRQPVATPTTSARPRGHLTVSRLSAPQPQPQPQPQPQPQPQPQPQPQPQPQPQPQPQPQPQPQPQPQPQPQPQPQPQPQPQPQPQPQPQPQPQPQNGHVAPGEYPAVRFRAPQNRPSVPASASSTNPRTGSSLSGVSSWASSLALHIDATPPPVSLLQTLYVSDDEDSDATSLFLSDSEAEALDPLPGEPHSPITNEPFSALSADDSQEVTRLQFGPPPVSANAVLSRRYVQRTGRSALAVLIRACYRLQQQLQRTRRALLHHSDAVLTSLHHVRMLLG

SEQ ID NO: 37 - protein, 3122 aa, human herpesvirus 2
MIPAALPHPTMKRQGDRDIVVTGVRNQFATDLEPGGSVSCMRSSLSFLSLLFDVGPRDVLSAEAIEGCLVEGGEWTRAAAGSGPPRMCSIIELPNFLEYPAARGGLRCVFSRVYGEVGFFGEPTAGLLETQCPAHTFFAGPWAMRPLSYTLLTIGPLGMGLYRDGDTAYLFDPHGLPAGTPAFIAKVRAGDVYPYLTYYAHDRPKVRWAGAMVFFVPSGPGAVAPADLTAAALHLYGASETYLQDEPFVERRVAITHPLRGEIGGLGALFVGVVPRGDGEGSGPVVPALPAPTHVQTPGADRPPEAPRGASGPPDTPQAGHPNRPPDDVWAAALEGTPPAKPSAPDAAASGPPHAAPPPQTPAGDAAEEAEDLRVLEVGAVPVGRHRARYSTGLPKRRRPTWTPPSSVEDLTSGERPAPKAPPAKAKKKSAPKKKAPVAAEVPASSPTPIAATVPPAPDTPPQSGQGGGDDGPASPSSPSVLETLGARRPPEPPGADLAQLFEVHPNVAATAVRLAARDAALAREVAACSQLTINALRSPYPAHPGLLELCVIFFFERVLAFLIENGARTHTQAGVAGPAAALLDFTLRMLPRKTAVGDFLASTRMSLADVAAHRPLIQHVLDENSQIGRLALAKLVLVARDVIRETDAFYGDLADLDLQLRAAPPANLYARLGEWLLERSRAHPNTLFAPATPTHPEPLLHRIQALAQFARGEEMRVEAEAREMREALDALARGVDSVSQRAGPLTVMPVPAAPGAGGRAPCPPALGPEAIQARLEDVRIQARRAIESAVKEYFHRGAVYSAKALQASDSHDCRFHVASAAVVPMVQLLESLPAFDQHTRDVAQRAALPPPPPLATSPQAILLRDLLQRGQPLDAPEDLAAWLSVLTDAATQGLIERKPLEELARSIHGINDQQARRSSGLAELQRFDALDAALAQQLDSDAAFVPATGPAPYVDGGGLSPEATRMAEDALRQARAMEAAKMTAELAPEARSRLRERAHALEAMLNDARERAKVAHDAREKFLHKLQGVLRPLPDFVGLKACPAVLATLRASLPAGWTDLADAVRGPPPEVTAALRADLWGLLGQYREALEHPTPDTATALAGLHPAFVVVLKTLFADAPETPVLVQFFSDHAPTIAKAVSNAINAGSAAVATASPAATVDAAVRAHGALADAVSALGAAARDPASPLSFLAVLADSAAGYVKATRLALEARGAIDELTTLGSAAADLVVQARRACAQPEGDHAALIDAAARATTAARESLAGHEAGFGGLLHAEGTAGDHSPSGRALQELGKVIGATRRRADELEAAVADLTAKMAAQRARGSSERWAAGVEAALDRVENRAEFDVVELRRLQALAGTHGYNPRDFRKRAEQALAANAEAVTLALDTAFAFNPYTPENQRHPMLPPLAAIHRLGWSAAFHAAAETYADMFRVDAEPLARLLRIAEGLLEMAQAGDGFIDYHEAVGRLADDMTSVPGLRRYVPFFQHGYADYVELRDRLDAIRADVHRALGGVPLDLAAAAEQISAARNDPEATAELVRTGVTLPCPSEDALVACAAALERVDQSPVKNTAYAEYVAFVTRQDTAETKDAVVRAKQQRAEATERVMAGLREALAARERRAQIEAEGLANLKTMLKVVAVPATVAKTLDQARSVAEIADQVEVLLDQTEKTRELDVPAVIWLEHAQRTFETHPLSAARGDGPGPLARHAGRLGALFDTRRRVDALRRSLEEAEAEWDEVWGRFGRVRGGAWKSPEGFRAMHEQLRALQDTTNTVSGLRAQPAYERLSARYQGVLGAKGAERAEAVEELGARVTKHTALCARLRDEVVRRVPWEMNFDALGGLLAEFDAAAADLAPWAVEEFRGARELIQYRMGLYSAYARAGGQTGAGAESAPAPLLVDLRALDARARASSSPEGHEVDPQLLRRRGEAYLRAGGDPGPLVLREAVSALDLPFATSFLAPDGTPLQYALCFPAVTDKLGALLMRPEAACVRPPLPTDVLESAPTVTAMYVLTVVNRLQLALSDAQAANFQLFGRFVRHRQATWGASMDAAAELYVALVATTLTREFGCRWAQLGWASGAAAPRPPPGPRGSQRHCVAFNENDVLVALVAGVPEHIYNFWRLDLVRQHEYMHLTLERAFEDAAESMLFVQRLTPHPDARIRVLPTFLDGGPPTRGLLFGTRLADWRRGKLSETDPLAPWRSALELGTQRRDVPALGKLSPAQALAAVSVLGRMCLPSAALAALWTCMFPDDYTEYDSFDALLAARLESGQTLGPAGGREASLPEAPHALYRPTGQHVAVLAAATHRTPAARVTAMDLVLAAVLLGAPVVVALRNTTAFSRESELELCLTLFDSRPGGPDAALRDVVSSDIETWAVGLLHTDLNPIENACLAAQLPRLSALIAERPLADGPPCLVLVDISMTPVAVLWEAPEPPGPPDVRFVGSEATEELPFVATAGDVLAASAADADPFFARAILGRPFDASLLTGELFPGHPVYQRPLADEAGPSAPTAARDPRDLAGGDGGSGPEDPAAPPARQADPGVLAPTLLTDATTGEPVPPRMWAWIHGLEELASDDAGGPTPNPAPALLPPPATDQSVPTSQYAPRPIGPAATARETRPSVPPQQNTGRVPVAPRDDPRPSPPTPSPPADAALPPPAFSGSAAAFSAAVPRVRRSRRTRAKSRAPRASAPPEGWRPPALPAPVAPVAASARPPDQPPTPESAPPAWVSALPLPPGPASARGAFPAPTLAPIPPPPAEGAVVPGGDRRRGRRQTTAGPSPTPPRGPAAGPPRRLTRPAVASLSASLNSLPSPRDPADHAAAVSAAAAAVPPSPGLAPPTSAVQTSPPPLAPGPVAPSEPLCGWVVPGGPVARRPPPQSPATKPAARTRIRARSVPQPPLPQPPLPQPPLPQPPLPQPPLPQPPLPQPPLPQPPLPQPPLPQPPLPQPPLPPVTRTLTPQSRDSVPTPESPTHTNTHLPVSAVTSWASSLALHVDSAPPPASLLQTLHISSDDEHSDADSLRFSDSDDTEALDPLPPEPHLPPADEPPGPLAADHLQSPHSQFGPLPVQANAVLSRRYVRSTGRSALAVLIRACRRIQQQLQRTRRALFQRSNAVLTSLHHVRMLLG

SEQ ID NO: 38 - protein, 3288 aa, Cercopithecine herpesvirus 1
MAQRGDRGIVVTGARNQFAPDLEPGGAVSCMRSSLSFLSLVFDAGLRDALSAEAVDGCLVEGGAWTRASAGSDPPRMCSAVELPTFLEYPSGRGLRCVFSRLYGEVAFFGKPAPGLLETQCPAHDFFAGPWARRPLSYTLVTIGALGMGLYRDGDEAYLFDPHGLREGSPAFVAKIRAGEVYTYLTYYTQEHPEARWAGAMVFFVPAGPGPPATAALTAAVLQLYGASETYLQDEPFVERRVVVSHPLRGDDVAPAGAVAVGEEAGRAPAAARKAPPQTPPPKAAAPEPAGAADAGVWGAALAGAPLALPAPAPSDSAGGDDAEDDEDGAMEVVSPLPRPNQHYPLGFSKRRRPTWTPPSSLEDLSAGRHHPKRASLPTRTRRSARHAATPFSRGSGGDEQTRPAAGPRPPTPASRPPTPGAPPTPGAPPTPGAPPTPGAPPTPAGPTTASSEPPTPAGPTTASSEPPTPAGPTTASSEPPTPAGRPPTPAGRPPTPANPTASSEPPTPAGRPPTPAGRPPTPANPTASSEPPTPNPEGAPAPSSNEQPPAAASTDEATQKALDALRDRQPPEPPCGSLTELLGRHPDTDGGVSRLAAHEAGIAREVTECSRLTINALRSPFPGSPGLLQHCIVFLFERVLAFLIENGARTHAGAGAEGPASGLLDLTVSLLPRRTAVGDFLASTRMTLADVAAHLPLIQPVLDEGSIVGRLALAKLVLVARDVIRTTDDFHGELAELERRLRATPPTEVYARLSEWLLERSKAGPDTLFAPATPTHPEPLLQRIQALAGFARREEVRAAAEDREVRGALDALARGVDAVARRSGPLTVAAVSPEEPGEGGGRPHPLSPEAIRVRLEQLRADGQKAVEGATREYFHRGAVYSAKALLAGDARDRRYHVASAPVVPVVQLLESLPAFDAHVQEVARRARVPAPPPLATSPPAELLRELVQRGRDLEAPADLAAWLASLGDAAGQGLVVRKELDELAQAIYKINERTVRRSSGLAELERFEALDAALRGELESEAAFEPGGGDGAAAGGLPAETRRLAEDALHQAKAMAAAKLTDELSPEARERLAARVRAIEAMLEEARARAEAAKAALARFFQKLQGVLRPLPDFGGLRVAPAVLATLRADIPGGWTCLPDAAQAAPPEVRAALRADLWGLLGQYRGALEHPTADTAAALSGLHPNFAEVLRDLFPAAPETPLLVSFFSDHAPRVAQAVSEAIAAGSAAVATASPESTVEAAVRAQGVLADTVAALSPAVRDPACPLAFLVALADSAAGYVKATRLALGARRAIARLGALGAAAADLAVAVRRENPQADGDRAALLEAAARAVEAARAGLAACEGEFGGLLHAEGSAGDPSPSGRALQELGKVVAATRRRADELEAAAADLAEKLADRDARAGRERWAADVEAALDRVENRAEFDAVELRRLQALAAQNKYNPRDFRKRAEQALAANAKTATLALEAAFAFNPYTPENQHHPALPPLAAARRIDWGPAFGAAAETYAEMFRVDTEPLARLLRITGGLLDLAQAGGGFIDYHEAVSRLAEDLNGVPSLRHYVPFFRRGHAEYLELCDRLDALRADVHRALGGVPLDLAAAAEQTVRLRGDPAAAAELVRTGVTLACPSEDALAACVGALERVDQAPVKDTAYAEHVAFVARRDLGEAKDALVRAKQQRAEATDRVTAALREALAAHERQARSEAESLANLKTLLRVAAIPAAAAKTLEQARSVAEIVDQIELLLEQTEKAAELDQAAVDWLEHARRVFEAHPLTAARDGSPDPLARLHARLDALGETRRRTAALRRSLEAAEAEWDEVWARFGRARGGAWKSPEALGAAREQLRALQTATNTVLGLVADAHYPRLPAKYQGAIGAKSAERAGAVEELGVAVERHDGLLARLREDVVARVPWEMNADALGRLLAEFDALAEDLTPWAVDEFRGARALVQHRLGLYSAYAKARAQTGAGGTPPPAPAPLLVDVRALEARARSPGERHEPDPRTVRGRGEAYLRARGDPGPLELREATSDLDLPFATSYLTPDGTPLQYVVCFPAVTDKLGALLMRAEAARARPPLPPEGLDSTQTLAAMCTVPLITQLQLALSDAQGDGFRLFGRFVRHRQPGWRDSAAAAAELYAALTATTLTREFGCRWDELGWERGAPAPAPLAEPAGTRRPRVTFNMNDVMVALVAGTPEHIYNFWRLDLVLQHEYMHLTLPAAWETGAGAILFVQRLTPHPSPEVRVLPAVAAGPPPATGLLFGTRLADWRHGKLSSSDPLAPWRAAPELAAGGAAAALGGLGGPRALVAVSVLGRMCLPSAALAALWSCMFPEGYSEYDSLDALLAARLGAGRPPDPQGGREDVPAPPAHALYRPSGQRVLVRGGAPDPAARVTVMDLVLAATLLGAPVVVALRSDPAFSKGSELELCVTLFDSRERGADAALREVVSSDVETWATDLLHADLNAIENACLAAQLPALSALIAARPLAGSPPCLVLVDISMAPLFVLWEQPDPPGPPDVRFVGSDEIEELPFVSPGADVLAGLAAEGDPFFARTILGAPFSLSRLLHETFPGAPVHRRPPEARFPFAAGAGPDPGGGAPPDPEAPPPPSHQTPAILPDEPVGETVHPRMLTWIRGLEELASDDAGDPPPPLPPAARPAAPDRSVPPPRPAPRPSEPAVQSRARRPDAPPQSARPRTPRDDPGPPAAPSTLPPSPPPPAPHPPDPPPPDPSPRPNELGGGETAAAPSAPDPPPPQTVPAPERPRDDRAPAPGRVSLPRRASRQGRPAPSSPLQRPRRRAARPAVGSLTNLADPHPPGPETPTPTSPTANPHATTASATPAPPPVAATGPTRPTSPATPTPTVAAAGRASAPPAPAAPAAPAAPAAPAAPAAPAAPAAPAAPAAPAAPAAPAAPAAPAAPAAPAAPAAPAAPAAPAAPAAPAPAAVPVVVPAPTATLTATTTTSPAPAATPASPVPTPTSSLPTPPSKPPAFFQPSLATGGSVAPGGDFRRRAPSRPTAAVPAAPSRPPARRLARPAVSRSTESFALPPDELARPRTPEAPAPPTETEEAPVAERPAPPEPPQGRPPSPAAPDAGPAAASGPSGGVPAPRLGALVPGRVAVPRRQIPPPAPPREIPAPSPPPPRSHAPRVSSWASSLALHEEPDAGPVSLKQTLWPPDELDDASDDSSLDSDPERLDLGSLDPETDPETDPFAHPPDPRAPEAGDRASPSSYFGPPPLSANAALSRRYVRSTGRSALAVLIEACWRIRRQLRMTRHALLNRSGAVLTGLYHVRMLLG

SEQ ID NO: 39 - protein, 3247 aa, Bovine herpesvirus 1
MAFAPVARPQPGEARAGGAAGGLRINGAARAVPPSTPVEQRSGTMSGVAGARAPATPADVAVAAVGFRNQYDAALGPGSAVACLRSSLSFLRLAFAGGVDAALGADAIDGALAEGAAWARESGARPEMCSIVHLPNRITDQAGAGSGLCCVFSRVYGECGFYTPPAERALSTQVPAREFVDAVWQPRRTSLALVIVGAMGVGVFRAGDAVYLFDPHGSGDVPQAFVARMPPRALYAYLALRACGQPESRWAGALVHFVSAGPEPARPEELRAAVSALYGASETYLDDEPYVERRVVSVHPRAPANTACLTAVAVGGAGAECEDPTFARPGSPALLPAEPVARMPPQITAAAAARAAGKRTSLPRRRRAPWTPPSSREDLRAPAARARAGKPPRKVRGAETAGAGEDWGEGGNAAAARPPPALGAAAPAHAATACAAELGARAAEVTAALDAVQAHAASIAADADACVARAVRVARPRDAAAERDPLEHAIAGLFERVLAFLVQNGARTRHDAPSAVQPLFGAVAQALPQHTPASAFITSSGMVLEGLAAYTRLSEAMRAQPSALGDLADAKLRLVAAAVAADTERLHDAVDELEGEVDGAGGAGPLELCGAVSARFMERLSASGRRLFSRAALRDGRPLDARVRALFERARAREARVAAETAALAREILALEGTVRAARERLDAVPPDDPRAVPADSAVADLAAELAAGRAAVVARSAAALSAELDALAAEALRQYFLRGAQYSARAILADRGGGERFRVASAAVAPLQRLAGSAPDLAARVARLGAAPGAAAPPLPRSARKGELLRDLLAAGATAHTEDGLAAWVALLHEAQAEGAAAKKELDAVLSDVAAINGRSASRASLEASMARFEAMSAAAARAAAGAAAEAPGAGAEEAADAAEAAVRGADDVLRQLDALATGAAADREARARVAAARAGLEAQALAARRRVAELGERRAELYQRLDALLLPLQGFAGLRAAPGALERLCADAGAQSAEDCARFLQEAPPRVAAGVQDRLWQLFGRYREALENPSALAPGALAGLGPAFAAVLGTALGQAMGPAARAFFEGHAERVAAAVARAAAEPAAPAAVAAAASALREAAEALRAGGAHVPPEFAFLDALQAQYAARREAQTGARRLGAALAAVSEASGALASAASALKNAMTAGADAAETTAAAAAADAALGAAEEVLRAADEAAVAAGGEDADGGEDASEADADGAGDAPPAGRLGPQAAKLHADLRKARALARRRAEEVRRLRAEAERRAEGAAAQRQEERWREDLRAALERVEANSAFDAAELARLRAAAAARGYDPRPLARQADRALEASAQRATAAIEAVLAFNPHAPENAKIDVPPPLAQLRGIAWWDAFSLAAPALSALFPAADVGQLTRLMHIATGLLTFAASGGGEPRYYDAVTYLEGDLAAVPRLAKYVAFYRRGHAAFEEERARLGALRADVLQAAGGRAAEISRARRGNVLRSPAERRALEAGVRLELPSEALIASALAQLERFDHARFAGSAYEAQMLSTVRRDAAAAREALEAAKAARAEATARAERILGEVVAAEAARDRDEGAGLANLKNLLRITPPPAALGPALDRAASAADVVTQAALLLAAVEEAPELDVAAVEWLQQARSIIDSHPLTARIDDRGPMEPFAERIDALRELRRHLDALRRQLESAAAAWDGAWELFVAARRRAAASREDHEGAKARAAALQAAAGVVLGLRADEHYARLPAAFTGALDARLAERGDALGAFHEAARAADAGLQRLQATLAAVRGEAAYEGLRALLAAFDAQVAELPAWAAQAHAPFRDLLVLRMRLYEAYFKLVPRAAARARGAAAARAARAAATVHSRADPATRAVAARAARLCERDLHLRGRVAALLGDEAAVCTLREADSEIDALAPRAYLDADGTPLCYRVCYRAVGDKLAVALCSQVGASMRPPLDESGVVEAASVAAVNVLNEIVNLRLEIERARDADFGAFCRFVRHRRADWGLADVRSAAAELYAGLLATTLPRRHGVASLGALCFSLAERGLRPARAPASGPAPSGGGALELTPADLVVAALMGAFMHLVNFTRLDLVQKQEYMCKTLDGVLTEALTSRVAVNSLAPGEPGARRPVPLGGGADDPADGALFAIRAADWDAGRLSPSDVLAVWRHAPDAATRATVAELARLIPGGALTTISVLARMCIPGDLLAALWTTLAVDSLGAQTQSYDAFLARRLDAPSTVHATGGPSEDAAALAAAAAAAGGRPALFCPTGSSVTFTLVRQPPSEVQTVNALDLVTCALLLGAPVVVAMENPDVFSEGSRLIMCLRLFDTRPGGRDADAPAAVSSDLNSWGERLLALDENIIENACLTAQLEQLSALIASKPLRSAPPCLIMLDTHLQLARVLWARAAAPPLAMLKIAEDDVLRDLPSLTLDYEDALPPAASAADPLFTHIISENNVPDFAAASGDPLYAHPPVFQHATDNLFPHARAAAGGGSGNRVANARAVAAAARPPTPASAPAPAPALAPASAEPALAWGADEWLDDEIGDAYPPAPAHDAYPPAPAHDAYPPAPAHAQGKPALLAAPAAGAAEAPGVERARRLTRRTGPRKSMPAALPWRRPPPASLVSAAPPVPPGPRLPPAPPLPPPAPPLPPPAPPLPPPAPPLPPPAPPLPPPAPPLPPPAPSTAPVPAPPLPPPALTPALTPAPTPAPTPAPPLPLPAPITVLVPAPVPAPAPIPAPAPTPAPAPTPAPPLPPPAPDGAMGALSATRRPTRRAGARKSLPAAQPRQRLLRSRSPASVPAPGSELVPPPSGGALGSPPSFVPSRPPSLEPVPGLPLPPSRVQAPVDAPAPPPAPERPAPPPAPERPAPPPAPERPAPPPAPERPAPPPAPERPAPPPAPERPATAVQSGSKIPAPSNALVGQVAPALLRRGPSPRGKPGAPARPTGGANIARKNTRAVDPTVIGSGSALSGLGSAEEDADFGASGMYVAPPGPAPLAAPPAPSALPAPRLEAPAAASLPAPPIAPPIAPPIAPPIAPPIAPPIAPPIAPPIAPPIAPPAMPAPPSAAPATAGSSAPPIAPPATPAPLIAPLAAPVPRLVGAAARGRVIGHMRAAAPKPRGLRAAERPPMRSWASRGNLHTQDLLASAEARICELPAPPKVPLGADSDATDSSLDGSGPETEEETEAEAGGDGPGQDAPKARVVPANSLLKRQYLRGTGISVLALLLEACEKIARRLRATRDVLRQRAAEVTADIFALRLLLG

SEQ ID NO: 40 - protein, 3204 aa, Bovine herpesvirus 5
MSGVAGARAPATPADVAIAAVGFRNQYDAALGPGSAVACLRSSLSFLRLAFAGGVDAALGAEAIDGALAEGAAWARASGPRPEMCSIVHLPNRIADRADADSGLCCVFSRVYGECGFYTPPAERALSTQVPAREFVDAVWQPRQTSLALVIVGAMGVGVFRAGDAVYVFDPHGSGDVPQAFVARTRPEALYAYLALRACGQPESRWAGALVHFVSAGSGPARPEELRAAVSALYGASETYLDDEPYVERRVVSAHPRAPPVTACLTAVAVGGAGAECEDPALARPGSPALPPAEPVARMPPQITAAAAARAAGKRTSLPRRRRAPWTPPSSREDLRAPAARARAGKPPRKVRGAESADPAGEGWGGAGVGAGEDTALAPRPPPALGAGAGEDAVAACAAELGARAANITAALEGVRAHAAAIDADIDACVTRAVRVARPRDAAAERDPLEHAIAGLFERVLAFLVQNGARTRHDAPSAVQPLFGAVAQALPQHTPASAFVTSSGMVLEGLDAYARLAEAMRAQPSALGDLADAKLRLVAAAVAAATERLHDAVDELEGEVDGAAGGGDAGPLKLCGAVSARFMARLSASGRRLFSRAALRDGRPLDARVRALFERARACEARAAAETAALAREILALEGTVRAARERLDAVPPDDPRAAPADGAVADLAAELAANRAAAVARSAAALSAELDALAAEALRQYFLRGAQYSARAILADRGGGERFRVAAAAVAPLQRLADAAPDLAARVARLGAPPGGAAPPPPRLARKGELLRELLAAGATAHTEDGLAAWVALLHEAQAEGAAAKKELDAVLGDVAAINGRSASRASLEASMARFEAMAAAASRAAAAATAASDGAGGGGTGPLGPGAEEAADAADAAVRGADDVLRQLDALATGAAADREARARVAAARAELEAQAAAARRRAAELGERRAELYRRLDALLLPLQGFAGLRAAPGALERLCADAGARTAEDCARFLQEAPPRVTAGVQDRLWQLFGRYREALENPGALAPGALAGLGPAFAAVLGAVPGQALGPAARAFFESHAERVAAAVSRAAAEPAAPAAAAAAAAALREAAEALRARGAGLPPEFAFLDALLAQHVARREAQTGARRLGAALAAVGEASGALASAASALKNALAAGADAAETAAATAAADAALGAAEEVLRAADEAAAEAAGGEDAEGAGEAADADGEAVSAGRLGPQAAKLHADLRKAQALARRRAEEVRRLRAEAGRRAEEAAAQRQEERWREELRAALERVEARSAFDAAELARLRAAAAARGYDPRPLARQADRALEASAQRATAAIEAVLAFNPHAPENARIDVPPPLAQLRDIAWWDAFSLAAPALSALFPTADVGQLTRLMHIATGLLTFAASGGGELRHYDAVTYLEGDLAAVPRLAKYVAFYRRGHAAFEEERARLGALRADVLQAAGGRAAEISRALEEVTYVRSPAEAARALEAGVHLELPSEALIAGALAQLERFDHARFAGSAYEAQMLSTVRRDAAAAREALEAAKAARAEATARAERILGEVVAAEAARDRDEGAGLANLKNLLRITPPPAALGPALDRAASAADVVTQAALLLAAVEEAPELDVAAVEWLQQARSIIDSHPLTARIDDRGPMEPFAERIDALRELRRHLDALRRQLESAAAAWDGAWELFAAARRRAAASREDHEGAKARAAALQAAAGVVLGLRADEHYARLPAAFTGALDARLAERGDALGAFHETARAVDAGLQRLQATLAAVRGEAAYEGLRALLAAFDAQVAELPAWAAQAHAPFRDLLVLRTRLYEAYFKLVPRAAARARGAAAARAARAAAAVHSRANPATRAVAARAARLCERDLHLRGRVAALLGDEAAVCTLREADSEIDALAPRGYLDADGTPLCYRVCYRAAGDKLAVALYSQAGASMRPPLDNGGVVEAASVAAVNVLNEIVNLRLEIERARDADFGAFCRFVRHRRADWGLADVRPAVAELYAGLLAATLPRRHGVASLGALCFSLAERGLRPVRAPLGGGSGGGEGRGAPPGGGGALELTPADLVVAALMGAFMHLVNFTRLDLVQKHEYMCKTLDGVLAEALAGRVAVNALAPSGGGARRPVPLGGGADDPADGALFAIRAADWDPGRLSPSDVLAVWRHAPDAATRATVAELARLIPGSALTTVSVLARMCIPGDLLAALWTTLAVDSLGAQAQSYDAFLARRLDAPSTVHATGGPSEDAAALAAAASAAAGRPALFRPTGSSVTFTLVRQPPSEVQTVNALDLVTCALLLGAPVVVAMENPDVFSEGSRLIMCLRLFDTRPGGRDADAPAAVSSDLNSWGERLLALDENIIENACLTAQLEQLSALIASKPLRAAPPCLIMLDTQLQLARVLWARAAAPPLATLKIAEDDVLRDLPSLTLDYEDALPPAADPADPLFTRVISENNVPDFAAASGDPLYAHPPVFRHAAGDPFPHARPAAGGGSGNRAANARAAACPQGRTPAPAPAAPPAPAEPALAWGAEEWLDDEIGDAYPPDFAGAAYSPEPAEDGRPPDFAGAAYRPDLAPARTPAPYPPEPAEDGRPSDFAGAAYRPGSAGDGYPPESAPAPTPVLRPPEPAPTPAPASTRGAPPLAAAPAPEAADAPGVERARRLTRRTGPRKSMPAALPWRRPLPAAPPAAPRTHPPPAALPPTAAGAAASPASPPAAPPDSPRTHPPPAVPPPAAASTAASPASPPAPPPPPTSLSPGTAPPPPPSLPPELAASTAGTPGAQTVRRPGARKSMPAALPRRRPPPPFLPPSAGAPADGPPARGSGVDAGRAAEPAPPLPAVSPPGPPAVSPPPRQPRPSPADAGPPGAAPAPAPPAPPAPPAPPAPPAPPAPPSQPRADGPAPPGPPIAASRNVPAAPAPPRAPPTVLAIPAPPKPRPAPKGRPGAPAKPAAGAKAARRHARAVDPTVIGSGFALSGPGPADGEDADFGVSGMYVPPPDPATQPAGPAPAPPSVAAALPAAARPAPATQPAGPAPAPPSVAAAPPAAARLAPPAQPAPPTRIVPAAPRPVDVGGRARPTARPMARLAAPRPRESRPAAAPPMRPWASRVDLHTRDLLADAEARIRTLPAPPEPPVDPGLDTADSSSDWSEPETETEAEETEAREGRGPGRDSRRARVVPANSLLGRQYLRGTGISVLALLLEACEKIARRLRATRDVLRQSAAGVTADIFALRMLLG

SEQ ID NO: 41 - protein, 3084 aa, Suid herpesvirus 1
MTADAVVVGYRNQYDPDLGPGSGVSCLRSSLSFLRLVFTNGVEAALTADAVDACLREGQAWTGPGGGMCAISELPNKIVYEGAGGAALHCVFSRVHGECNFFTPPAEGFQSTQITARAFFDGLWAQRAESFTLVVVGAVGLGLYRRGDVVYVFDPHGHGQITQAFVARLRPADVYAHVAGYAASELRWAGAFVFFLPHDQFAAVPALYGVGETYLVDEPYVERAVPSVHPMALPPDPAAAAPPTRGPGAIPVLAVAPVPAVPVSAAAAPAPAPGGAPLPPPPRVQKRPSMPKRRRPPWPASQEDLSSTHTVGGRPSRKFRPRTREPMGFAVRELELAPLDLGPLQAHADELEEHVVACAGRSLFPGRAGLLERFAELLFGHLQDFLVENGARTHGEAPCAVAALFEPILGALPERGPAGDFVAATHMVLTEARALLDVAGDDASAPVAKLALVAARVARPANEAMHRALDALESGGSYAQLSACFMDAAARDPGGVYDQTVVARARALFARARARESRESQRALERARDVSALEAALEAAHANFDAVDAGAPGAGGAAEPNTRDVKERAAKLPRKLALMVGDAIREYFLRGAQYSAKAMLMDRNGAQRFRVANAAVAGLRRLVDSLAAAEERLRALDAAPAAHDGALLAQLLEAGEDVSTDERLGAWFALLAEAQAHGHLERRELDTLTRDVTQINERAAKRASADADLARFDAMDAALAAAPDEETRVRAMEDLLRHIRAMGGHVLSQETRARLARRDAELRTLVQAAQQRAAEQNDLYMRLRALLGDFATLGPAVDLLAHITATPVPGTVLAALWRLVARYRELLTSDPGALVGAGQPLGALVNALADNPPITRFFLAHADAVAAALADADTAEKAARAAGVLRAAVEHPELGFLEPLARQCEERAAALRESERVAALQAALSAAVAAAVEMLGRLRAQPDEGTLVEARHLALEVARAAAVEEPQLQKMALAARQRVGELEAAVAAVEARRAAEVERAQRQLWARAADAALRRAEQHAFDAAEFVRLRDVAADRGYDAAELRPAAERALGANAETVTTALDAVFAFNPHLPENAGAAAGPPLQALQRLTWLDDYAVAAPVIGSLFGADTEPLSRLARIAGGVLARAAAGMGRFSYYDAVGFVGADMAHVPRLAKYVDFYRRGFPEYEAELAALGARRADVLQAGGSRGAEICRALEEVTYVRAAADAARALDEGVHLHVPSTELLRGAVTYLERFDFARFERTAYAEAMRALVRRDLAAARDGLAAAEADQAAATERATRLLREVVDAAAARDRDGALELANLKNLLRVTPPPASLNIDRAASAEELVTQAALLLGAVEEAPELDVQAVEWLHQARLIIDTHPLTTRVDGRGPMDEFAERIEALQTLRQEADTLRQRLTAAEVAWDEAWEDFGRARGRAGASEDEAPLAEARARALQTATMEVLALRSDAVYPRLPAKFVGRIESKYSARAGALGDFFRDAGAVEEDVRELDRMAARIGPELRHDALRALAAAFDARAERLPRWIPPKYAAYRELLTLRLQFYAAYGGAGPTPARPRASTAPCARLKQRVAARLGDRDVICTVREARSDADAVFPTVFLDDNNVPVEYQVCYRAVGDKLAAMLCTDEGAELRPPLTEGLMETATVAGMRLMNEIIHLRLNYEKARDTGFATFARFVRHRRADWDSAAKAAAETYAAVLATTLSRTFGALDRVAHPPLEPAVRLALPDLMVAAVAGEPTHLFNFTRVDLAGQHEYMAKTLDAVLDRALDAVDTLDAGARPPRRPLRDGQALFTLRVADWTEGRFSETELWAPWAHTETGQRLARAVPGALATFTVLARMCIPPRLLSALWSALQPEANAAYDAAVTERLDAAAHVREAAALGAPGAGDLYAPVGTGMTFTVTGSPPSSATRVSAMDLAAAAVLVGAPLVIAMENLDVYSPGSGLTLAVTLCDLRDPALHRGVSTDLSSWGERLLALDDNPIENACLAPQLERLSALIAARPLAGEPACLVLVDSQLAVSRVLWAPPEPPRRPGFTLEDDPLLREIPFLETTEDQLPEMDPADPLFTSIIMGDARVDPATSEELYGRPSFRNLGDPFPYAVSATPTRPAAPIPGLPPELAGEDDGFVPDDDFGANPWLDLLGDDLVASALPLPPSPPVDPPTLPPLAPAAPPAPPPAKPAELTPAAKLAPPAPPPAKPVETTPLTKPQPQPQQGPPAAHKKPAAGTKAAAAPKQQPREPAPKPHSSPRKIQPSLKARIEPPPPVINPPYPATAPAPETAPPEAPQAQPPAAAKPTPQPQGPPPPPQPPSAQAPPAQKPPAQPATAAATTAPKATPQTQPPTRAQTQTAPPPPSAATAAAQVPPQPPSSQPAAKPRGAPPAPPAPPPPSAQTTLPRPAAPPAPPPPSAQTTLPRPAPPPPSAPAATPTPPAPGPAPSAKKSDGDRIVEPKAGAPPDVRDAKFGGKVAGAASPGRPLDDERYKAGRADKCEHTVGPDQHRTRSPPGTSDSEPASASSSAASTPSREAASSSSSPAGTPSDGARGGPPSAAGAAARAPPGAPGSGGQRRGDGGPAADASRGDQPAGHLPAAPVAQPLPSAVLRTKARTSVGIRDHSRRKSMALVPRRPVSAPRQTGGLPPAAQPEGRPPGGLEPRPPQAQAQAPATAPTGPPAAAAAAATAAVPAPGVGPGGPVRRAAGPRPPAGHLRLHIPPPQPVQLEGVVVPLPASPETPAPAQTQPPRSPLAAPTSLAAPSEIERPAASAAAAAAATTTTTSSSSATTSSAPAAPPPAPPPSRPAAPPPAPPPSRPAPTPERHGEPPRLMFTPPPLPPQPSQQRPPEAPWTWPEPRDSAGPPALFTPPPSDPPVQFPGREEPPLPPPPPPQPLWASEGEAETPATPLRSPLERMPWPPPLEAPGTTPELPPEFAPPAPRSPLADPHQPPAWFDDLTALVPPAHPPPRRQQQRPPPPASPLGGLGRVWPTREDLTREPAEEAPELDPWGLEDEEGDEDGDEDVSLAQELLPPRVVESDTLINRRYMRATGLGALALLIAACRLIARRLRETRTTLKGSARRFNVDLFQVRLILG

SEQ ID NO: 42 - protein, 3421 aa, Equine herpesvirus 1
MAQTLVPANKAGGAQADVVVIGYRNQYDSQLGEGSHVSCLRSSLSFLRLIFTHGIDFALTADSIDGVLVEGRAWTVAGSKSGEAPCMVSIVELPNKITYANSANALCCVFSRLYGDSGFYMHPGDGFQSTQIPARQFFDGVWKSRSESFALITIGAIGLAVYRHGDVAYVFDPHGHGSVTEAFVVRVLARDVYAYLTGYAATDPESDWAGALVFFVTCGPTESEPGFLISATSLLYGISETYLSDEQYVERSVATSHPGISTPPPLTDVAVGAVSEAWQYQELENGAATLDADMEGVAPAAAQVRASVIRQPTEKRVSLPKRRRPPWTPPTSSENLTTSGNTHTVAGRPSQKVRNATANVQNPTTGNGSAWAEALNDGGVDNASRPGQAVGAAGTLQNPAPGDALAMETTQASEEALRTRRVFRLSGEDEAPYDLGDAVGVLSAEINELATRAEELDVLSSTCVDSTVWVTRPHNSPDMDILEQFITMIFNRLLSFLVENGARTRTDSPSVIAGLFPGVLAAIPTQSAVVNLLQATGMALSDVASYKSILNMVSNEDSPVGELAVIKLELVASEVIKSTQKLVARVEELERDVTSGSVNPLGLYTYLTERLVAEMTKHGGDLFAREPKPGAVSLTERIGSLFRKARTREARATRTNASLARDLNAIEAAVHAAHDKFDAIEIKPADPSDTTNMDELAKSLDLSAVPTRVAKVIKKVESMVSDSIREYFLRGVQYSARAIAMDKTSGARFQVASAAVSNLERMLDSLPNFEKSLNSVVASAGIQGPPPAQISGSRKATLLGNLLRAGQNLTTDNALGAWAALLSEAHTEGHIERRELEAVIKEITSINDHAAKKASVEADMERFRVLSAAVDQATSDMYNSNPHALDTIIRGAEEMIRQAKVVEAHFDSGRISREAASRVGVRKREVETLANSARQRAAEISAARDEIYSRLQSLLLPLAGFVGLRAAPGVLEQLAKDAQRSTSEELRNLMHEAPKQVVSTVHSHLWSLFGQFREALEHPNSTTSSALAGVGPAFAIVVRSLLDPNKQRESVEFFITHADALADTVGAVEANPNSELAVAHAVNSIAAAIQTVSVGGRTITEFAFLVPMLERYQSRLTIVRETQRLATAQRAVAASVSAAAEVTTKLRAVAVPGVQEDVLKAAIAAAKHVSSEVTAAATAAERELARLDSKALSVAQVARAHQDLQKQTAVAKQRVGEIEEVLANLNKQQRELQDRAVHDRWKSDLLAALDKIETKSSFDVSELSRLRDLGAARGYDSREFAKRAEQALAANARAVIAVLDNVFKFNPYAPVNSKKETNPTISMLYNISWWDDFTLAAPILNTLFAGVDVEELMSLMRISTGMITFASTNGGRPKYNEAVNSLSSDMLKVPQLAKYVDFYGKWYTEFNAEMDVLSKLRADVLQAVGVRSGEISRALEEVTYVRNAEVAEKVLADGVKLYIPSDALIAKAVKYLEEFNQKRFAGSAFEEAIATTIRQDLSTAREAATQAEAARSEAMHRATHILREVVEAAKAADRDASANLANLKNLLRLTPPPQSVAAALDKATSSDDIVTQAALLLGTVESTPELDIKAVEWLQQARSIIDSHPLTTKIDGKGPMDPYAERIEKLHTLRGELDELRRQLTATEVSWDEAWGNFSRAVPRADVSMDGFVDAHQRARTLQASMGVISEMRADNKYGRLPPKVIGAIESKFAERHKNLETFNDTSTVLQTAITQFDSLVQQIPPEMEYDVLRSLLASFDQLAAVLPKWVGAEYAAYRSLLLMRIGLYDEYQKIAGIAAAGSRPHLEAVEYRSAVEDANLRRASRVSSLMGDKDVILSLREAKSSIDTAFPQVLLDAKGVPVEYRVCYRAVGDKLAAMLCGKLGVSMRPAMPSDPIVESSSVSGINVTHDILQLRFGLEKAYHSGFSTFARFVRHKRADWSPTEPAQAAAEIYAAVLATTLTREYGATWHRIRFMASSGLFVASPDSVCDTQGGRGKKSNNIVHLTLSDVVLSAMLRNSMHLVNFMRLDLTRQHEYMARTITPVLTKSLLSDILINTLVPTDTSTQWRSLPLAGDLEDLAQGMLFSIRMSDWKQNSFSTTSLLDVWMRSPGESGRAAAAKIASAIPGNPLATFTVLARMCIPPNALASLWEALQPEAFSQQNLSYDDVVTSRLDIASTVQTSVAVDPEMKSVDTKSRKQLYTTTGTSTTFTLAGSAPSAVKEVSALDVATCALMFGAPVVIAMETPEMFSEASGMSFCLKIFDSRPGATDHEIIQAVSSDLSSWGTSLLALDPNAIENACLTTQLEILSGLVASKLLAPAPPCLIVLDPSMRVIKVLWESESPPNDLVITLAEDEIIAELPYLNADDDLLPPMNPDDPIYTRVISGTNIPTATTEGSLFADQQLEFLRPESNPFPFASHDSSQSLDVPSSPSSGSDKYEEDPTGIVYDAPVDDMSDMAMNKAKAWQEWLEDGFAEDDYRELSNAMPAPPKTTPVVESKQKSDSVDRAPTLPPKAAPLPPSDASAIMSGKPVFKYTPGNKSAVPPSVPAPPTLPPAPPLPQSTSKAASGPPPTLPPAPPLPQSTSKAASGPPPTLPPAPPLPQSTSKAASGPPPTLPPAPPLPQSTSKAASGATQSDSGKTLTLDVPKTQSKDKVVPVPPTDKPSTTTPAALKQSDASKPPTAAIQHQQKLGTPVTPKDSGDKPTDNASAPVGVSPVTPDGTPGAKPPPKDAPPVDDTKQPVRKSLPSQVRGGRPYIRPSLGPFKFTGPPGYTIPVHGLPPSDSNVTQSTKEPPKPAVETPAAAPAKSAAAPAAAPAKSAAAPAAAPAKSAAAPAAAPAKSAAAPAAAPAKSAAAPAAAPAKDQTKSAAEVPKPAKDQAKDQAKDQAKDQAKDQAKDQAKSTTGQKLAKDPKSDGLTDDVALEIVPEKTPLPDDSPIGAVPENTPLPDDSPIGSPDLSASKNSHTTDAVSSDRFSVACKVPLPDSPEDDFYSYAVDVPLPDSPTDDPSSGRSDARAPTVGGVASIHRKSDSRNNRQSDAWRRAFADTLHGRPRNRSATKPCKSAPYKVPHAISYTKIPSVPNDQSGLAGKPCSEEPKRPTGRDTPVGSWNVSPSQAPADIPTAIPQNQNTSESPRTTSLKSPTRTVQSSMPADDIDELAEYDLQIARAVPVTKHPQPPPANQTPPPQEPPAPIDDRKNIRPPLSEEEIIAFLINMDDDDAGNASGPVDLHSVQAPKLPKQSKPTTNQFVPLDWWTETEPVVDADSLDLSPKQQRLFSWESTRDLLNINVRDRVYEEESDDEYTVSWDQHLVPAVSPTSVSSYSSDTVTDSYTDINDPRSVVCPLDGNAQNNVREFLDTHSSRVRVVPADELLSRRYFRSTSLSAMALLIAACRTIVRRLRATRRVLTDINRSLLLDLKQIRVLLG

SEQ ID NO: 43 - protein, 3534 aa, Equine herpesvirus 4
MAQTLPPVPTAGGAQADVVVIGYRNQYDSKLGVGSHVSCLRSSLSFLRLIFTHGIDFALTADSVDGALVEGRAWTVAGSKSREACMVSIVELPNKITYANSTNSLCCVFSRLYGDSGFYMHPGEGFQSTQIPARQFFDGVWKSRSESFALVTIGATGLAVYRHGDVAYVFDPHGHGNVTEAFVVRVPSRDVYAYLTGYASTDPESDWAGALVFFVTCGPTESEPNFLISATSLLYGISETYLSDENYVERQVETSHPEITTPPPITDVGMGSVSEAWQYQELDNGAAAQDTDMDASTPTATPVRASVIRQPTEKRVSLPKRRRPPWTPPTSSENLTTADNTHTAAGRPSQKIRTSTAKVSDVTASNNGDVWAEVLDDGGVTNAGISDQTLSNNVPDTPAHGDALAMETTRAADDVLKTRRIFRISGEDEAPYDLGDAVGVLGVEIEDLITRADELDVLSSACVDSTVWITLPNNNPDMDLIEQFITMIFNRLLAFLVENGARTRSDSPSVVATLFSDVLAAVPDQSAVVNLLRVTGMALSDVASYKSILNMVANNDSHVGELAVIKLELVALEVTKLTRSLVAKVKELERDVTSCTVNPLGLYTYLTEKLVDEMTKHGGDLFAREPKPGEATLTEQIGSLFRKARTREARATRTNAFLARDLNAIEAAVHAAHDKFDAIEIKPADPSDTSNMDELARSLDLASVPNRIAKVAKKVESLVADSIREYFLRGVQYSVRAISMDKTSGARFQVASAAVSNLERMLDSLPNFYKSLSSIVTSAGIQGPPPTQISSSRKAALLGNLLRAGQNLTTDNALGAWVALLSEAHTEGHIERRELEAVIKEITSINDYAAKKASVEADMERFRVLSAAVDQATSDMYNSNPHALDTIIHGADEMIRQAKVMESHFDAGRISREAVSRVSVRKREVETLANSARQRAAEISAARDEIYSRLQTLLLPLAGFVGLRAAPGALEQLAKDAQSSTSEELRNLMHDAPKQVVSTVHSHLWSLFSQFREALEHPNSTTASSLAGVGPAFAIVVRSLLDPNKQRESLEFFIKHADTLAEAIGAVEANSNSELAVGHAVNAISASIQTVTVGGSTITEFAFLVPMLERYRSRLTIVRETQRLATAQRAVAASVSAAAEVTAKLRTVAVSVISQDVITAAIASAKHVSSEVTAAVTTAERELAGLDAKALSVAQVARAHQDLQKQTAAAKQRVVEIEEVLANLNKQQRELQDRAMYDRWKADLLAALDKIETKSLFDVSELSRLRDMGAARSYNSREFAKRAEQALAANARAVINVLDNVFKFNPYAPENSKKETNPTISMLYNISWWDDFTLAAPILNTLFAGVDVEELMSLMRISTGMIMFASTNGGRPKYHEAVNSLSGDMLKIQQLNKYVDFYGKWYSEFNAEMEVLSKLRADVLQAVGVRSGEISRALEEVTYVRNAEIAEKVLAEGVKLFIPSDALITKAVKYLEEFNQKRFAGSAFEEAIAATIRQDLLVARDAATQAAAARSEPSTEATHILREVVEAAKSADRDASANLANLKNLLRLTPPPQSVAAALDKATSSEDIVTQAALLLGTVEATPELDVKAVEWLQQARSIIDSHPLTTKIDGKGPMEPYAQRIEQLHTLRGELDELKRHLAATEVSWDEAWGNFSRAIPRADVTMDGFVTAYHRARTLQASMGVISEMRSDSKYGRLPPKVIGSIESKFAERNKTLETFNDTATVLQASIAQFDSLVKKIPPEMEYDVLRSLLVSFDQLAAVLPKWVGAGFSAFRNLLLMRIGLYDEYQKIAGIAAAGSRPHLEAVEYRSATEEDNLRRASRVAALMGDRDVILSLREAKSTIDVAFPKVLLDAKGVPVEYRVCYRAVGDKLAAMICGKLGATMRPAMTREPIVESSSVAGINVTHDILQLRFGLEKAHQSGFSTFARFVRHKRADWSPTEPAYAAAEIYSAVLATTLTREYGATWHRIRFMSSVGQFTTDSHSGSESHVGKAKKNRNIVHLTLSDVVISAMLRNSMHLVNFMRLDLTRQHEYMARTMTPVLTKALLSDILINTLVQTDASVNWRPLPLTGTPEDLAHGMLFSIRMSDWKQTSFSTTSLLDLWMRSPGENGRAAAAKVASAIPGNALTTFTVLARMCIPPDALASLWEALQPESLSQQNLSYDDVVTSRLDIASTVQTSVAVDPEMPSVDNTAPKQLYIPTGASTTFTLAGSAQSAVKEVSALDVATCALILGAPVVIAMETPEIFSEASEMLFCLKIFDSRRGATDHEIIQAVSSDLSSWGASLLALDPNAIENACLTTQLERLSGLVASKLLSASPPCLILLDTSMRVMKVLWEPESQPQELIITLAEDEIIAELPYLNTDDDMLPPLNTSDPIYTRVISGTNIPTAMVEGSLYAGQQLEFLRPDSNPFPFALLNQQPLDVPSSPSSCSDKYDDDHTGILYDTNGDDMSNTAMNKAKAWQEWLEDGFAEDDYQELSNAVPIPTKTAPESKRSLGLPDKIPPLLPPKKAPLPPSTASDILAGKPVFRQPHNNKSVVKPLVTSSSTVSPTPPLPAATEKLSSINTQSPSDKNIPPSNTKTQPPDNRLPVPSENNLPHFVPQTPAPPTDTSKPCTVIQSQQNLGTPAPQKEPEKKPTNNASTAVGSTNKTTDEPQVVQPPSKNASEANNIKQLNEKSLSKPWRPSIRPSLGPFKFTAPPGYSIPMDGLPPPDPNEALLTAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPAAAPAPSKPQNTLVAIVAKDQAKDQAKDQAKDQAKDQAKDQAKDQAKDQAKDQAKDQAKDQAKDQAKDQDLTKQKSNPAFKTGFETTPLPNTSPSGAVPENTPLLDDFPIDAVPENTPLPDDDSPIGAVPENTPLPDDDSPIGAVPENTPLPDDDSPLGSPPHQPVSKTLHNTNLVSSDRSAAAANVPLPDSPSDGFYSYAVNIPLPDSPTDDEPFSNQSRAQASAAGSVSGSSYKINTGTGRIPTAWQRAFAHTSHGRSRNRSTSKPSQSAPYKVPPALSYTKIPAVPNAQSHHAGKPSNEKPKCDTGPTVLFGSRNISPSQTSTTANISSTLPQNQSTAKSSHKVAKKTPLRVVPSSMPADDIDELEYDLQINRAVSNTKPLPKSPLQQPQPEYSSVTTDYKQNVRPPMSEDEIIALLINMNDNTENDAEPIDIKSIRAQNLPKQIKQAANKFVPLDWWTETESAADADGLELSPKQPKLFSWESKRDLSNINLKDKIYEAESDDEYTISWDQHLVPAVSPRSVSSSSSDTATDSDTDTNNSSSVLNSLADNTQNDASELVDTHSSRARVVPADNLLSRRYFRNTSLSAMALLISACRTIIRRLRATRRVLTDINRSLIMDLKQIRVLLG

SEQ ID NO: 44 - protein, 2653 aa, Cercopithecine herpesvirus 7
MDIIPRIPTNVAGIGSYNQFDEQLGPASGVSCLRTSLSFLHLVYAQGIDVSVTSDAINGCLKEGAMWTSTTSTREGISPNMCAIVNIPNRISYESLHYNTSTCCVLSRIYGDSFFFTAPTDGFLCTQVPARVFFNNIWSKREESFTIMTVDSTGIAIYRRGDKSFIFDPHGHGAISQAVVTSVSTSDVYSYIAAEYTCRPDGTESQWAAGLLFFITANEGNVTEQSLVSAVTLIYGVCETYFTDETYNERLVVSKHPLLTAPPEPTSIVLNQTEPITEVSNDLSLEAVLHPTLTNAVVNDPRCEYTEVDPWHSIVETTKSSKLQNRRRRRPAWTPPSSDENLAGNKCDTSTCSTEVTIETPDKPARKIVKPVNGDDNETSLVLVTPDVPRVLDDHENTSLHITSTDTENIEPPSTVSPSVNVDTLKNRQPFFYKTNITQTVNNLVEICKDNTDVTFQETVDILDIVISEHTDMPIWITRPLSITEQDPLELFIRACIIRLTGFVVENGTRTHTDAQSVVSALLEPLLGILTSFPSAVELLKSTGLVLRETSGLSYAITNAINKKCPLGLLIVSKLTLVATSIQNATDLLHAELDVLEADLGACIPNELYTKFSTGLISIFKAATANKPDLFANTTPSRPGALLDRLRIICESLQVRENHCMRQRAAVVKEIEALEHAISLIRNKLDTIQLPDLSSEIQKLNYDDTDGLIKRLYNIRTEAANLVTKAISEYFQRGVFYSVRALAVERDSSTKFKVATASIAHIKRFIQSLDKLDAKVQSLFLAINSELSVESLTTLPGAVLLNNLVKEGEDVSDDTGLGTWLALVGEAQTAGHITRRDVEELIRSIKSINCRATQRASADAEILRFDTLSAAVDQAIGDYTDGKSSKDSNIENLISTLLKASDDLVRQAKVLNEPVLQSVLTPSTKKRIEIRVKEVQTFTADARTAHEVLKSRRNAAFVRLRNILRPVADFVGLRSSVDIFPEICVELNISDDVNDLRNVVLSAPVDVRAQLLNDFWAIIEQYRDTLESPGNARTSLLAGLGSCFVTVIQIIPIPNEYREPMLRFFGEISDYVSSALAKIANSIDSEEDIKSVVSILERATSLVSSVPALTFISTLYNKYSNILKHSANIRRLSHLQKQLNDEFAALTASCEYLKTQASLSDINQEHFNNAVLTVKKTMSALMDITICVEQELTNMPSESISNGSTILRVTKDLYKMSVTAKSRMQEVDAYIIQCEKTFNERTNQSTKEHWLRDITACLEDIETKFIFNSTELARLRDVAAEGGFDVHGVYSQARQSISACENCALVALNAVLGHNPYAPENEQIPPPLSLLRDLTWFDDFSVTSPVFATLFPGINIEPLMLLMRVRAAILLSADVALNGIPRYRDVVLQISGDLLRIPQLSKYFDFYTRSYDHFEAEISALSELRADVRQASGARFAEVTRALEEVTHVKDYNTAKTVLDKGVYIVLPSEVLVVKAIQYLTAFDHKRFIGTAYEKAMKLIVDRDLTTANLELDQFKMMRQSATERAKHLLQEIIQNKDVITQQHMVDFTNLKSLLRLTPPPPTFVAAIEKANSPDELVTQFALLLSALEQSKESLDVKAVDWLYHARNILDSHPLTVLIDETGPLSKYADRIDALYSLRVKLDVLEKRIEAAEISWDTAWQSFKQNTGVNSMSVNAHNEALDNIAALEASTNVINLLCSEMEYNYLPLPLKSRITQKQQERLNSLHEAVIMENHFKKNFNELNAVTERILTEGDHESLKRLLESFDTIIKLLPVWVKTSYVVYRKLVLLRLGLYDTYAEIITPQSIYGCFNTFSFTPSNFKDEYHIQNIRSRVAAFMHGSTVVQTLNECKSDIDLLFGPHHTFYVDSDNIPLQYRICYKSVGMKLGAMMCHPQVISMRPKLASGGVVEQTTLSALKVINEIIEIRLDYDSVVMTGFNTFSTFVRHKHPDWATSQQRRAFVEIYAGLIVNTLTSKFGVTLNRMVYSFNTNQLTSISGTKAVPVKQNVRSIKITLSDVINILVATYPAHLLNFARLDLIQQHEYMAKTLIPTLESAFRGRLLVQSLKPSDKKYVISNPPKHLLPLAFASSHDPTGGRLFAFNYTDWKTVKISAKFSDPLIPWNTSPYTSVKTAVVNITNIITTDVLITVTVLGRMCLPPKALVSMWNTLQPEDFIYQTQDDVDNVVTARLNYASTLTANFNAHLSNNFKHPNMSAEPYVPTGTSAVFTVTGTISTPLLSVTAFDIATAAILFGAPLVIAMETAPVFSPDSGLTLCVKLFDSRQDATDFGISLAISPDVISWGLPLLNMDPHPIENACLTAQLEKISALLASKPLANTPPCMLILDEHMSPVKILWEPVDTITPINDITFYDSDHFYDLPYITLDSDSFPGYIPDDPFFSQLIGGYAPYGPPNLDKINFYPRAPVFKHHHQLVLKSQPPSQPPSQPPSQPPSQPPSQPSQPSQPSQPSQPPSQPSQASQPSQPSQASQPPSQPPSQPSQPSQPSQPSQPSQPSQPSQPKAITKVPLYVQAQEEHLNGYNLISYSPTTISTVPDINKMVHNKAISRENISSSVFITPTREETRPDNYWVDMQGLSESHAAPPNYTSTQVQTENRSVSTTYVPPWLEHESNCSDMSNVLPKGMTQMLNADVTPSIQLF

SEQ ID NO: 45 - protein, 2763 aa, Human herpesvirus 3
MDIIPPIAVTVAGVGSRNQFDGALGPASGLSCLRTSLSFLHMTYAHGINATLSSDMIDGCLQEGAAWTTDLSNMGRGVPDMCALVDLPNRISYIKLGDTTSTCCVLSRIYGDSHFFTVPDEGFMCTQIPARAFFDDVWMGREESYTIITVDSTGMAIYRQGNISFIFDPHGHGTIGQAVVVRVNTTDVYSYIASEYTHRPDNVESQWAAALVFFVTANDGPVSEEALSSAVTLIYGSCDTYFTDEQYCEKLVTAQHPLLLSPPNSTTIVLNKSSIVPLHQNVGESVSLEATLHSTLTNTVALDPRCSYSEVDPWHAVLETTSTGSGVLDCRRRRRPSWTPPSSEENLACIDDGLVNNTHSTDNLHKPAKKVLKFKPTVDVPDKTQVAHVLPRLREVANTPDVVLNVSNVDTPESSPTFSRNMNVGSSLKDRKPFLFEQSGDVNMVVEKLLQHGHEISNGYVQNAVGTLDTVITGHTNVPIWVTRPLVMPDEKDPLELFINLTILRLTGFVVENGTRTHHGATSVVSDFIGPLGEILTGFPSAAELIRVTSLILTNMPGAEYAIKTVLRKKCTIGMLIIAKFGLVAMRVQDTTGALHAELDVLEADLGGSSPIDLYSRLSTGLISILNSPIISHPGLFAELIPTRTGSLSERIRLLCELVSARETRYMREHTALVSSVKALENALRSTRNKIDAIQIPEVPQEPPEETDIPPEELIRRVYEIRSEVTMLLTSAVTEYFTRGVLYSTRALIAEQSPRRFRVATASTAPIQRLLDSLPEFDAKLTAIISSLSIHPPPETIQNLPVVSLLKELIKEGEDLNTDTALVSWLSVVGEAQTAGYLSRREFDELSRTIKTINTRATQRASAEAELSCFNTLSAAVDQAVKDYETYNNGEVKYPEITRDDLLATIVRATDDLVRQIKILSDPMIQSGLQPSIKRRLETRLKEVQTYANEARTTQDTIKSRKQAAYNKLGGLLRPVTGFVGLRAAVDLLPELASELDVQGALVNLRTKVLEAPVEIRSQLTGDFWALFNQYRDILEHPGNARTSVLGGLGACFTAIIEIVPIPTEYRPSLLAFFGDVADVLASDIATVSTNPESESAINAVVATLSKATLVSSTVPALSFVLSLYKKYQALQQEITNTHKLTELQKQLGDDFSTLAVSSGHLKFISSSNVDDYEINDAILSIQTNVHALMDTVKLVEVELQKLPPHCIAGTSTLSRVVKDLHKLVTMAHEKKEQAKVLITDCERAHKQQTTRVLYERWTRDIIACLEAMETRHIFNGTELARLRDMAAAGGFDIHAVYPQARQVVAACETTAVTALDTVFRHNPYTPENTNIPPPLALLRGLTWFDDFSITAPVFTVMFPGVSIEGLLLLMRIRAVVLLSADTSINGIPNYRDMILRTSGDLLQIPALAGYVDFYTRSYDQFITESVTLSELRADIRQAAGAKLTEANKALEEVTHVRAHETAKLALKEGVFITLPSEGLLIRAIEYFTTFDHKRFIGTAYERVLQTMVDRDLKEANAELAQFRMVCQATKNRAIQILQNIVDTANATEQQEDVDFTNLKTLLKLTPPPKTIALAIDRSTSVQDIVTQFALLLGRLEEETGTLDIQAVDWMYQARNIIDSHPLSVRIDGTGPLHTYKDRVDKLYALRTKLDLLRRRIETGEVTWDDAWTTFKRETGDMLASGDTYATSVDSIKALQASASVVDMLCSEPEFFLLPVETKNRLQKKQQERKTALDVVLQKQRQFEETASRLRALIERIPTESDHDVLRMLLRDFDQFTHLPIWIKTQYMTFRNLLMVRLGLYASYAEIFPPASPNGVFAPIPAMSGVCLEDQSRCIRARVAAFMGEASVVQTFREARSSIDALFGKNLTFYLDTDGVPLRYRVCYKSVGVKLGTMLCSQGGLSLRPALPDEGIVEETTLSALRVANEVNELRIEYESAIKSGFSAFSTFVRHRHAEWGKTNARRAIAEIYAGLITTTLTRQYGVHWDKLIYSFEKHHLTSVMGNGLTKPIQRRGDVRVLELTLSDIVTILVATTPVHLLNFARLDLIKQHEYMARTLRPVIEAAFRGRLLVRSLDGDPKGNARAFFNAAPSKHKLPLALGSNQDPTGGRIFAFRMADWKLVKMPQKITDPFAPWQLSPPPGVKANVDAVTRIMATDRLATITVLGRMCLPPISLVSMWNTLQPEEFAYRTQDDVDIIVDARLDLSSTLNARFDTAPSNTTLEWNTDRKVITDAYIQTGATTVFTVTGAAPTHVSNVTAFDIATTAILFGAPLVIAMELTSVFSQNSGLTLGLKLFDSRHMATDSGISSAVSPDIVSWGLRLLHMDPHPIENACLIVQLEKLSALIANKPLTNNPPCLLLLDEHMNPSYVLWERKDSIPAPDYVVFWGPESLIDLPYIDSDEDSFPSCPDDPFYSQIIAGYAPQGPPNLDTTDFYPTEPLFKSPVQVVRSSKCKKMPVRPAQPAQPAQPAQPAQTVQPAQPIEPGTQIVVQNFKKPQSVKTTLSQKDIPLYVETESETAVLIPKQLTTSIKTTVCKSITPPNNQLSDWKNNPQQNQTLNQAFSKPILEITSIPTDDSISYRTWIEKSNQTQKRHQNDPRMYNSKTVFHPVNNQLPSWVDTAADAPQTDLLTNYKTRQPSPNFPRDVHTWGVSSNPFNSPNRDLYQSDFSEPSDGYSSESENSIVLSLDEHRSCRVPRHVRVVNADVVTGRRYVRGTALGALALLSQACRRMIDNVRYTRKLLMDHTEDIFQGLGYVKLLLDGTYI

SEQ ID NO: 46 - protein, 3342 aa, Gallid herpesvirus 2
MTDSTDSRQATTNCRKYSRTTSNAPMLAANVLRDKSTSGLCSLDRKHDPYFGQIMDNPEVILDEWAKMVIDTTDVTVVAVGIRNQFAPDLSPASSVSCLRSSLAFLRIVFAYGLDTVISSDAIDRLLLQGKAWTIATSEDGTYTTCVPHDLPNRIISKDAGGNLCVAFSSSYGEFEFYLEENTPTILDTQISARTFIEQIWKKKRGDVYCLIVVGVLGIGVYRSGDGIYIFDPHGHGHIGQACIVRVSEGYFYQYLTSYADPSATPDWSATFVYFVSTVSICPPRDEIISTVSRIYGTSDIVLDLGRAREEDNRKVVSADFDPPSRPQPRLTKLVIGSTDTTIQGSDYPCIQAEDLNGEDSRPLDHNLSYNDAPTNTESVAPPSTKDCRDCINIQNPGETLDDTHSTIVDPSTKDSISVTAWQGVFSDVIEDPPPKTNFQFSFGTFAKVAENKIGTTSVEGCIRNYARHKRRRPLWTPQSSSENISLDGSSSSLSRKHSRKSKRTLESRIVEAVTSTESSDVTENVDTYPPVISNIPDEPTLGSSPTTSNRGEDTTVEHLLKNRPLFNFKSLTEDEDGLVQDRLWSDEYLSHYPLADIRDKIEDVACSIDSGLRIIVHVGSPYDSDGGLLYVCMMDIFARLFNYIIENGARTTSDRESVVGYEMAALLKAFTIPVYFTTFIASTGMVLSEASESIDLIERVLAENSKIGNLALSKMILVALEVEEVTDELHKSLDAIEKEVGTADPYGIYERMAAILVDTLYHNSGKLYSEKTSSNSNQTLTDRVISLCTLIRDIEAVAIRKAELILAEMEALEAGVRWMNTTLDAFIMGGSGSSPMIDAADIVAKTSSAVVTQRLGDIGKTVIDVVGHSLREYYLKVALYSVKALTASSSDVSRFKIVVTDQYEKINRFASSLSVIDDVMVLIASRSNARVPSPVSQAFESELLGNLLEIGSDLDVPEKLTTWKNLMTSMQVGGWISRRELDMLMKEIDIVNEKATRHETVLTELERLNELETRFGSYTDLDTTVELQKLDEAIKIGEDIVKLAIVLEDKKNATSLSSDVREKLRDTRRKNETFITHLRERYQEVKSTIEDLYSSIRKILRPLPKFVGLRALDSKVKVITESIPRGMGSFENFLASAPSDIIGSLQSDLWVLFIQYKTILSRPTTEVAAELSGLGVPFALAIRLVFGPQGSYPAASVFFGKHADVLSATIAAAAVEPMSVEKTMAVVSTLKAAISDIDRANAIAPPQGIMSISSTERSDAFLFLKALLSTAEIAADVANRGQHLESLIQSVRTILDNLITSNHKIRSLNPREVISENDTSVVASAKVEFSNAIQTVGNVTATLSTFEGLTYTSNPHIQRKIAELSKLIRSANQRAGELDIAIQTYEHNRISAERSRSEDLWISSINSLLLNAEVKSEFDAMEINRLEDAARTGGYDTIRYKSRAEKIVIAHARVLESSIESVLKFNPYSTQNMVHGLSPPIAALKSITWGDSFMTAAPYYTKLFGVNCDTVMDLLHISIAILRHANANSGNVDYYLLMGELESALKSYPNLVKYVNFYRSGYVKFMSFLAHLEQRRVEAHHASGRVALEISAALEDLARTHSPEGARRALEYGVSIIIPSVNTIMSIAEELKKDHVEELEGTAYSEYGAHLLRRDTDAMTSLIHRVTTAIEDAKTRGEAILKNLAEASYAADRESAELLANLKNLLRLVSMPSHIAKAIDRSETVNDIVTQAALLLTKVEETKELDSQTVEWLRHAESVIDSHDLTVRIDESGPMSIYADRIDALVNLSKRLEELKSELALAEVAWDDTWATFVHDKDRIDKSSEGFSSARESAARTKVSVNMINALRSNAEYPRLPAKIIGLIDTKYRDRVVVLDAFLTTVKEIEATQKQMEGLCEKIPSTFAINDLKKIYTQFEDIAKRLPKWYTKRVARYSRLLTLRLALYAGYSNTFEGNVGDPMLLPFDAGDANNGANHTSNVGVVNRYLKHRVASWIRPKVVATLQEAFSEIDSPGCLTYLDSTDKPLRYSLCFRTVGEKLAACLCEPAAIGIKPQIPIQPITTEETEAAAGMLSDIMTFRLGFVHDMANHLYSFTKYVRTKRHNWMQSDYIKALGTIYCALIAITLTRKNRSNLSDIYFIPGRRTPIVDKKELKKNAANGRGKQVVRLDPADVMVTIMANIPGHMLTFSKLDLIDQYDFMDKTIYEVMTDSISTVAFVNCLSVQLSKDNIPDPNCRPLSLTGSVWDPAGGSLFSVRYSDWRQGKLSDTDPLKLWEDLDGDAADGLAKIRAAIPSSLLTTTTVLARMCIPPTALAVIWSSLLPDGLEQNCKSYDDVVTARGDLASSLDVTTSLLSCSENKNISSITDSPNLYDLTGNVTTFTVVSTPPSRVLRVNAMDIATTATLFGARIVIAAECPEAYSSESGLSLCIRLFDSRHGSRGCFLEPTAVSSDMTSWGTKLLITDNNPIENACLGQQLEHLSRIVASKPLASAPPCLLIVDSGMAPIKVLWSKEILDPIPIIRLISEDDALISELPYVDAGIRKEPELANEHMVIADVQEASKFFSDESRIYPCPIYNKCVSPDLSRDGDADISSNRIDSEDDTYADSMGSGYLSIDPESMWDRVVENDMEGAQVQLLLPGDIIHHNDRHVSENMNYPQIGHLDISPNYRDPIDETSSNPPPFPKHSNLFPSDHDPTSESSSKPTPAPKPTPAPKPTPAPKPTPAPKPTPAPKPTPAPKPTPAPKPKPPPDPDFKPSPAPKPSPASKPTPAPKPSPAPKPKPPPDPDFKPTPAPKPSPASKPKPPPDPDFKPTPAPKPKPPPDPDFKPSPAPKPSPAPKPKPPPDPDFKPTPAPKPSPASKPSPASKPKPPPAPDSKPSPAPKPKPPPTPDSKPSPAPKPKSPSASKPLPVLFPNSDSKTSPVPNPNTFSASKIPPTSSIAEETKPCQSNLPAIPLITKPDSVKNGYTTLKTDDKRKGSQSRYRNEKSRKHMHNTHTAVYNTTFNWTGTAAASSRHDMELNQYSPGIVTFDNLIDKEGYRHESETIPRESYSEHRKDLDWMSTPTVIANASSSLITNNDYGGIGGIDLKKYRFKNGTGLLRQGSPTTRLHCRKNNSSRSITDILVGTASPTNEPSPLLQRLKAHTISGDKKANMDAGTIRGRLYDYSSFWPPCIQGACSTSPKTIDLKYLSSEQATVAYPKHDDTHHQTPTGLAPNDKHSDMHISSIITETDTKENSIPGYNNIPMRHSSESESLTSLDSDSDDSHLHVSGSTDTTTDGSSTSRVIPADALLTRRDFRNASRGALYALTKACKKVARQIVYVREQLRTKVATLAIELFKIKMILTG

SEQ ID NO: 47 - protein, 3064 aa, Gallid herpesvirus 3
MAASESRGSTTRPSKKSTRLQPSPSTRRLETSDNSRDSSVSNMQNRAMRSGTNMDSEVILDEWARMVVDATDFTLVAVGNRNQFAPDLSPGSSVSCLRSSLAFLRVVFAYGLDAVLSTEAIDRLLLQGKEWTIETSRDGRYTTCVPHDLPNRILSNEPDGNLCVAFSSSYGELDFYVDENTSTILETQISARTFIEKVWKQKRGDIYCLIVIGVIGIGVYRSDNGIYIFDPHGHGHIGQACIVRVSEGYFYQYLTSYADPSTAPEWSATFVYFVSMISSTPPKEEIISAVSRLYGTADIVLDLGRVNEEENIKVVLANLDPPYRPQPTTTRIIIGTSTSRSEDIKVAVDSRSDRSPVAAGDVDVRREMGYAEEDAYVLPPEEAPVDAICDITNHESIVNAWTTAFPDIGDDLSVLTRPDLPKTRENVVRLETDTKSSSPPKQSFERHKRRRPVWTPPSSSENLSVDGQGQSSSRKPSRKTKRCLTYTDNPVAPSISLSEQADSGSSTTHNIPGQEEATRPDNVSFNEFISSQARAEHFVHGQSLRDRPPFNPHTVAGEGDESLRDGLWTSEHVPSSILSDINANIDDLEQSIALSMGMTIHLSRSGDYDGLLDICGLDTFTRLFNYVIENGARTTTEIGSVVEAEMASLFKAFTRPAHFSAFIDSTGMAVSRAGESADLIESVLSENSKIGKLALSKLVLVALEVEELTDALHKTLDSIEREASTADADRIYERMATSLVSAFNSNPGKIYSENTSLHAEQSLTDRVASLCRFIRDRETVARRKAELILAELEALDAGVRLMHASFDAFIMGGSGGTSGSRDDESIAKVSPSVVTQRLTAIERAAVDAVGNALHEYYLKGALYSANALRANVASTSRFKMVVVEQHEKMARLFASLSVLDDVIALVAARSGAQAPPPVTTAPESKLLGDLLENDSNLENPENLAVWKTAMAAVQAGGWISRQEFETLIKEVDAVNNNAARRETALADLDRLHELEARVASYTDLETTADLQKLEEVMKDVVELVKLSNALGGDKFANLLSSDDRAAIARRRLDAETLIAALQYRYEEIRTLYSSIKLMLRPLQRFAGLRALDSTVKTITQSIPGDTGPLETFLALAPPDVIGTLRTDLWLLFSQYRTILSHPSTSTAAELSGLGIPFSLAIRVILGPIGGYPAASVFFGKHADTLSAAVAAARLEPMSVEKTTAAVTALQNAVSDVNRATADTPSTGIMADLPAGQDDAFSFLTVLLSDAEVAAGVAQHGDRLDGLHRAVRAALNSVIASITKVKTSDPKTAAADSDASVIMSARTEIAHALQSARDANTAIAALESHAHVTDPHIQRKIADLAKSVGLVNQRTVELEAAVTTYERNLISAQRSHSEDVWVSSIHSLLSNAELKSEFDAMEMSDLEKRAVVAGYDTIRFRARAEKIVMSHGRAIHDAIETVLKFNPFSTPNRILPPIAFLKNMTWLDAFIAAAPYYTKLFGVSCDALLQLIKIVLAILRHADARGGNLDYYTLLGEIEPDVRAVPTLAKYVDFYRNGHEEFTGFLARHEQMRVEALHAAGRVDIEIREALENLARMYSPEGARRALEYGVSIVIPSANAILTITNDLEKEDITAFNGTAYAEYSAHTLQRDIEAVSSIKLRVTTAIEGAKSRAEVILKSLAEVSYAADRETAEQLANLKNLLRLVAMPPHIAKAIEKAETANDIVTQAAILLSKVEETKELDTQTVEWLKHARSVIDSHELTSRIDESGPMTIYAERIDALADLASRLDELKSELALAEVAWDDTWSTFMHDKDRMDKSSEGFAAARDSAARAKVSSNTINALRDNAEYARLPAKIIGLIDAKYRDRAGVMDAFLAALKETESIQKQMDTLCAKIPPTFVLNDLSTIASQFDILSKRLPKWYVRHVTRYSRLVKLRLALYAAYSAILPGVAGCPSLLPSDTGLNADANNRPAAGFVDRYLKHRVAAWIRPSVVVTLQEAFSEIDIPGIHTYLDASDAPLRYSVCYRTVGEKMAACLCEPSGLSLRPAIPSKVMSTRDMVVAAGMLGDIMALRLGFVQAAADNFRAFTKYVRTQNYDWLADGTVKAIGSIYCALMAITLTRKTGASLSDIFFLPGQVAPAADKKGLKKIAANGRPVRLELADVMVAIMTCMPGHVLTFSQLDLISQYDFMDKTLYGAINESLSTTAFINCLSTQLSKDAPSDSNCRPMSLTGGAWDPSGGSLFALRYSDWKQGSLSETDPLKPWEGVGGATSTGLAKIRAVVPSNLLTTTTVLARMCIPPTALAVMWSSLLPAEIEQSCRSYDDVVTARGDAASTLDVTTSSVLRPADKTIPKGSDGPNLYEPTGVAMTFTVTSTPPSGVLKVNAMDLAAAATLFGARIVIAAECPEAYSSGSGLNLCVRLFDSRSGSEGCFLEPAAVSSDTTSWGGKLLADDSNPIENACLGQQLEHLSHLIASKPLSSAPPCLVIVDTGMVPVKVLWPKTVFDPIPVIRLTSSDDAVLSELPYIDAGIRVETGASYDDIAVLDRQDAANFFSDESRQYPIPVYNKSRDGDVEDVLSIGTASEDEELPGNDFSRDRESMWDGMAAEKDVYDNAETGFLFHKELNDRSDRVVPENVECPSTVSTDASDGASSKLAFHYKLAPPNNETLSPSLLSPATQKLVVTKKTHNEQSTYAAVGIEERQKGRPVKHIDVSERSHDMPKPVYRGRLKFTDTAAACNAPGCALDQKSPAVVISDNSVRDDRKKHGTPNIPHRSDSDSTSTPFVVASSTKSLIPDDEDGGIGIRALKPSRAGTRIDSIGRISDKGAAVVRPNNASRPRCDTLGGLASVRRDSSPLLQRASPAVITADLNLEPVDRSVDISTAPGGKKQAAVGSPAHDGLYDHFAFAPPDVILADLRRRVVRFWETFCRDPRTSSSQSKPINTCRRSPCKATTAKHDHHNMYRGFVVTEAEAGQNKQQDSNIDPERYLFALESLSSVDSNTDGESQSSVPDSDRSSTDESSASRVISADRLLTRRDFRQVGRSALYALTRACEKMARQIAHARQQLRARIDTLTIEIFKIKMLLTG

SEQ ID NO: 48 - protein, 3112 aa, Meleagrid herpesvirus 1
MEAVRANMYKRNSLLESTAGDSELMLDRWTKRVVDTTDFSIVAVGIRNQFAAELSPGSSVSCLRSSLAFLRIVFTYGLDTALSADAIDGFLLQGKDWTIHTSDRGVYTTCVPHDLPNRILSKDPGGNLCVAFSSSYGESEFYLDDSTPIILDTQMSARTFVERVWKQKRGDIYCLIVIGVLGIGVFKTEDSVYIFDPHGHGHIGQACIVRVSDMYFYQYLTSYADPSGMPDWSATFVYFVSTAVSAPPKEEIISAVSRLYGTADIVLDIGRADENDNLKIVSADFDPSSRPRPRQTKIVVGTPKLVSEPEPADSHADSSPKLHSESTHLTPHEHGEYDPSTLVGGDSTNINISDPPARTDCRRYSEGSVIHESVNSHIEDVSEATSVPAVWSDASEISEEYSHLGAVAHDPSKNGHDHKPDRRSRGSNSRQKRRRPSWTPPSSSENVSSELPTFHQARRPPRKSKRALDLDYGHLSNEPPDVDGHNSDSPAGAISNISDSIEHLSLRNRAVFNPHTVTGNLDNTLRDSFWNDDYSGSYPLSTISDMIDSITESIADGMRLVVPTSHRDGGVLDVCIMDVFTKLFNYIIENGAQTVTDRASIVEPEMLALTRAFATSSHFSTFLSSTRLSLAEASESANLIESVITEGSRIGKLALSKLTMVALEVEEATITLNKSLDAIEREASTMEPDRIYERMATVLVDTFYRSGKLYSETTSYNSDQTLTNRVVSLCEFIRDRENMATRKAELILAEIEALDEGIRWMNTKFDAFVMGGGGGMSPAKGLSAIDMTATDAVAKKLESLGKAAIDIVGNALREYYSKCALYSAKVMRADMKETSKFKIVHTEQLERMTRLLSSLSIIDDIILLISSRCDARVHVSVSKALESQLLKDLMEIGPHLDVADNLVTWKGLMVSVQTNGWISRRELDLLLEEVDAVNDNAARRETAFTELERLHEIESKIASYTDIETVVDPTKLDEALKLANSIVSITKGLDGAKLASSLSADIREKIRQKRSETELLIARLSAKYAEVKAAVDGIYAAMRKLLRPLQNFAGLRVISSTIKTISDSIIPGMGSFESLLASAPPDVMGALRSDLWVLITQYKTLLARPSTGTASQLSCLGGAFALAIKVILGHESEYHSASVFFGKHADVLSAALTSAASEPTSIEKTSAAVSVLKEAIADINTVDETISQQDIVLSTERADMFSFLHALLTDAEGAADLASRGAYLSTLIRTTRDSSEAVIDAITKIKSLDPKTYTYNTDAEIIVNAKAAVSAATKQAGDCKDALTALDNEPLTYNSHIQRRITELNKLIESVNNRVGDFDIALQSYQRNRVDVERSRGEELWTSSVTSVLVNAEIKSEFDAVRINSLAETARDAEYDTVRFKSRAEKIVSAHARVVENAIVTVLQFNPYSTTNIIHGLKPPIVALKNITWGDAFFAAAPYYTMLFGVNCDVLISLLKVLFAILRYASAHPENLDYYFLVGTIESDLRAIPSLAKYVDFYRRGHDSFEGLLTRLEHMRVNALHASGRVSIEISDSLETLARTHRSEGARRALEYGISIVIPSATTIMSIADALQQEKITELEGTAYAEYSAYILRRDNDAIKAITQRVEAAIEAAKSRGESILKDLAEASYAVDSETAEQLANLKNLLRLVAMPAHIAKAIDKAETAQDIVTQAALLLTKVEESKELDTQTVDWLKHAESVIDSHDLTVRIDGSGPMTMYAERIEALVRLENRLAELKSELALAEAAWDETWSAFIHNRDRIDKSSEGFSAAQESATRAKASTNAINSIRNSMEYNRLPSKIIGSIDSKYKDRTVMLDNFLDSMKEIEDTIRQVEIICSKVPLTFSLNDLRAMSSMYDDIAKRLPKWYVKQVGRYVKLIKIRLALYTAYANASTESIGDPPLLPFDTGRADIAPDTAPSVRLADKYLKHRVAAWMKPKVVATLREAFSEIDMPGLTTFLDSTDTPLRYSICYRTACDKLAACLCETSAVNIKPRAPTNLISTVEMESAAGILNDLMILRLGYVQACADRFQLFRKYVYTGIHDWSPDYCIKAHGTIYCALIATTLTRMTGSNLSDIYFIPGQYGATIEKGEVKKFMANDRRRQVVRLDPADILVTVMACTPGHVVAFSKLDLRNQYGFMAQTLINVLTDAISNVLFVNCLSTRVVDGGKVDPNCRPLSLAGGEKDPSGGALFAIRYSDWKRGRLSDHDPLAPWGDISGDEGAGFAKIRAVVPTDSLLLTTTVLARMCLPPTALAAMWSSMLPDGIEPTCKTYDDVVMARGDGAATLCVTASTIDHTGVINGQTNLDCPSLYEYTGTATTFTVVSTPPSRKLKVNAMDIAACATIFGARIVVAAECPEAFSPDSGLSLCIRLFDSRSGSKGCFVEPGAVSSDITSWGAKLLAADSNPIENACLGQQLELLSHLIASKPLSEAPPCLVIVDAGMVPVKVLWAKEELEPAPIIRMTCADDALIAELPYIDSDVYGEDFQHMHVLDAQEASRFFTDDSRIYPTPIYHTGVDQHNTTDGGESINFFKINPGVCPLTGSEELPVYSDGEQDGYIGGGIEGNAAPDSVEYSALDIDDEGEMTPVNTNMTADVYARDIEPGVLVESHTLKPDYDPANPTSKTMTADSDLATQGDQDGFVSDVGSAMHYKPIVGMEFKPEPTPPIVYEPPAPETSRMESAPLKKLGALDKNVTAMAQSIVQPDNSSIKKNTDIRLAGRRTMPADNNGLRHKPKTGMSETTIKNVESYPSPPRWVRTAASQKRHGEIAGCVSPPIITLQNATPVNMKEGMGIRLEKHSELYDRLDEHQTAPEALEMPPIISAPLISPTPIYKELSGNWHKNIERMGRKQINSRSKERMASKASNSPLKSKISSSTADILVGMASTTSSHDHSERLSPPPIITPQLRRADDMYVNIKNIPVPEVKEEGNWKECPERNNHVPEVETRTLENESPPPPPNVIASWKQILPHTPRGNAHRKAVSQTTHKPPNLSSYRGPVLTEAESERKFNNVRVDPIHKRSESESVSFTDSDSDDSRSTIYNPNSTDTDMSSTSRIVIADTLMTRRDFRKASRGALYALTKACEKIARQITQTRDQLRSRVIGLAADIFKIKMLLMD

SEQ ID NO: 49 - protein, 2059 aa, Human herpesvirus 7
MRIIAGSTNQNDPKYGPRAGKQCMSNCFSFLHTVYLNGINNVLNKESIDIIMENGALLDNISTTTLKLETGNIPEYRFFTEIPKKISSNFGETIHELSRPFNGTLESQHIDNEVYLGLLDFLLYGKNKKPAFIVITIGVMARAIFIVDELFYLFDSHASDTENSAAIYICEDIDELYALLAIENVAEFYYDAVFSYFIETTDLSLEDGDATILILKTYKDPDIALSLNDFLTMYSSTSSTKTAETNTLISKQSPSKRKQEKTSLNSNSLEKKRKQGSSLKYYNNEVDLVPSFYELRPQFNNILFELSNFPIVKENVNWTLYIQKFATKSTQPFTKPFIWNRVFHLFSQVVDALIMIKNDHWDETQQQKQFFTHFLPFKEFSEEFENAIEACRENNLDLILLYKNYLSKTTAFKNLERILLTKFSAIVSPVHEKHYTLVNTWLTNLIQKLVKHPEDTNAFINDYVLKNPLNHFICLNKKEKQSIALLLNKKRMSMLKDVEIEKNGFVQLQAFIENIGEAPANYLDPENARKVNVEEVSEKDIPTLSTDKVSIPNESMFTSNKKHSIEKLIHAKLKAILSTMGQRLTRIIQENYNNIAAGFLPVNDLNNLFAYLVKLYFDVYSITINGFVVENELIKNIEQIYDNTQYLRFGLTRFNMQNLTPFTISVRKMFLDFFLSQKTLIDRAEEIIENLEFKSVTPEGKQKLATKNMLREQLEQLNAMDVDDTINLKTDTLTHQVLFSDQELRMIQDFILQLSIHNIPSINFVKSLKLHIILEKRPDILLALQEKVQNILYFYFQDLVNEIPAQENVLSTMLFIIELFPADSRIHLLETGYISRHIVKKWLNMKSLQDAEDLIRFININKEQLGKFEHQPFGKEIQKLIEKIHLFYKQKVIEYQEDVWSEMAKNIILTSPSELSQFLASAPTQRIIQKHKNNLDQKLLIHMENQAKQAMEDDKKRVACSKINLERHLNDLLLLLKDRQFASIQASVLIVCENIFKTIPDDNLIIQFSHALLSVLLDIEKDLKSYSSEILEKILINRPLETSRLLVFKDAYGNLKEFLNALKQSLFATADVQNKADFLIQILDFTYKFRHKTNKGKLLHSIYNEDFKLYEETLTELRKKATDAKESLTKLFKASEQKIELSRTIPLKEIYLNIETVNFQGYGNVVFRESAFKRAIEVEIKNYEMKLNDLIKHFNSHLKTKIDHIQILNLSFDNKWKDFVSKSKISFPPELTISSQELIKDPIKVITETLNKASNDLAYVISEKILKWLIVFVKELNTFFVATMSEFGEVIPFDYKHFRALEYEINSKYIEIENKIICNEIIENTDNIEKLSTLIKQIDPNRIAGGKQKFQDYLSKILTAETNQQQTRYKEQLKKQYFDLLDNIAHFRFAFDFNHQQNLILKLKDKFKTLRTDTVFERFPNLDDTFVSSMNVENFLQALEALSHFVQAAQNFLQNVLTEQADLFPQTNFIPVELSTVKTIPKSDINLRMKIHTPQTFFQVDSVFNTQLIVDEKGIPVQFYNVFHNIVFKFFALNYKKIIVPDKVLNLVSTKYKILTTLKSILSVVKSFWKEIINFDLTSYFQGKAEFTFQNVFPIINLKIFIYIITQAWSVTSDETQHSFELPLEKFSLLIIANNPEFLFGSLQCPVDLAINSLIPLLEKKKYFTAFTISDNPPKLSMDELKIVCLDLNTWSEITLEKYTFKKNSLMQLCMGKEKFFIYLLSALVLPQNFLNYIWIQYKPSCCAQDSFQQLIQDLCFEYTHQNHIKPISLNLQEPNALKHGERILSKFVLEKNANTSLFSIFLGKQFLLDYLLFSYLTATEMTFSYYVDSIKNFLLTIRHLENVQQNVDFRTILQSRNFDLKYLLTQSWTQNVLEQSIFHVQLDKIIADIKQPQLSLKKIPLVLFNGDNEVVSTYVPPEQASQTEQSFRIKNIFPNPVQEYSSKNVILFTNYPKNTKFLFNSPPPKTAAKSYKLPDTTDDINTETLSSPTIQRIPIKGLVPKENEIVFLPEKNTAHTDSKETKTHLIDTFNILSQTKGEIKTFSTDFDQTISKLKHLYF

SEQ ID NO: 50 - protein, 3195 aa, Psittacid herpesvirus 1
MWPPGLGTVVAAASRSQFDAMYEEMCYAMCVETSAAFLRACELAGPQAVQSQNVLDTILDQGAEITRTSVACETLPGGVKRYNMIDCQELPRLWRGSGQEPDMLLLHTTTFGNIPYDSTFDYEEKVLMLNGQQLGEKAAAMAREGAAAVLVIGNRGLGVAFTGGRHDPVFVFDPHGWMGGAAYMSRLPNPTSLTGFIADYVERRSGIVVTLTFLLWLYPDGWNIEQESPELRVEVISAALRAISQPPDFVFLDQFSETVVSPALPVPMGALRADRFAAKSIRAILARGTAPSPKKKPDSGEHGLPAGRPKPPKRHTPRSQNPALLDSTEKLSNLSPVKVKKPNKGKKMSVIAEPPSAAARTEQRGPGDSLGALAERPPWRLQGVRAAADVIAVPAASALAGLPEKPSDKLRGVLKYLPEAPTGGLSLGSGNALWAAILGTRVASLTDRLLVFLVENGITIRKAESEVGFLLDPVLAALAHRPDRGAVASLIGDTRLNLFALVARKAALLRLTELRDDRVSAVLVHKVQQVSSAIVSDTKTISAKLGALVDEISSEHPADAYGTLEKELLAFSLEKTAVVERPDETTAPILELVERAAEIVDAIEKETRAKAEREDRARRADEKLLAFAHSVWDEIDDIAGDVTRGVDVGSSQASAAASLAKRERIDIPEPSSMPPREDYSELKILDDKAKANAQKITDSAGKMLKVYANVIDYSISAFTSGTQSAIGRFALASPALDHMKAWLDRIAYADTLVDSLAGLTGRRESASPDWGRLSSLEPVRILEGLIETGADLSSDENLNHWTFQLFGAHAAGFMPSPSKWISAIHGINTRAHEVGLSATALAELETEIKAAEAMVADPGVRLECAKHALESAKAAVAGKSDPEQKARLAAAQARAAKLVDSCQRDLDEAKRLVDEATKRDAEIKKAAAALLRPVEKYQGLRGLGRSMAAAGLSDDAVAGIVAADTQVARVLRADAEAILANYERDFAELRGAQLLGTSAPLLRAVNFIDPRSGLGLLEPGSRIFLSEANDDLMDAVEDARTTRNTDSCSRAISALERIKWVIVEAEGAGQWPKFAVAAARALEGLRAKALIDDRAGLVRTTLAGMLKRAAIVSETVAKDTDDPEAAAERALKFVEMARRELRELELTDAEELSSQDYVALERALVELAEASRQKSIGLKKHAWRSRLRTLLERDRDDGEFSLDAWDQARDEGECYGGRDAVNDDLIKLARAVIDGRIQLGLRLVRAFFANNPYAAQASQALPGDARGPVELLESIRVAKWIFAFPGVADTYEYLFGISVVKLKALCEIGEEIVEAFDAAGSSDKNIDMHAFVQTVAGKLFQVSELTEFFDFYVRSYELFLDIRAALAETAGRVGALERTALEQLGAEERAAESIRDPEAAKERLERGDRDPDALTAMRELHGGLKLESKKQFEKTAYLEPLEYGYAEARRELERAIANVDAAKKLSQARVKDFLSSLLREREANDQELSRNLKTLKSVLAARSPKDVVAALNGAETLDAVVKIYADRLAEAEAENEAAIVGAETMEWLKFAAKTIDGSKMARETGGVGPTAAYAERLEKLCRARADADAKLKRLKDLYESFELALASAKEAGGKQKDESEDGWRRYEAAANNLLTSAEALGAQLRDEGNEQAGIKLLLLREPAAAAYEKGLENAAAVIKDVRTTLDDTSAGMRRLLTLYEAAKTEFAKPGLERLQKEISYAIAKYPVPKWFLALHAAVGKLVELRLGLYHAYEGLKISTIPYAPVAPESEYVMPDAALTAARVTAYMARSGKSVMTVTTHSLGIVGRAVVDEANQILEYKLCYATVSEKVAALWAAGSRLGAGQFGGLVLRDARDERGVEKFLGRGHAAVSLAATAAWLSGADTMITAELGSYVTFCATGHWPAMRDRKQLSMTVAACTTYCALAYATLTSTYGSAADTAVDSHGQFVPPEKFEAANTSGAVAAGAARGAKRKFALSIQDVLILLAACEPAHLTYFCRLDLLRQVEYMHKTLEAVLSRAVRDRVGVSCLEPPKADDTRKYMPVVMPAARGRFDKSYGACFAIDRHDWDSVKAPHYVGKMLEPWKTLPGTRENAERLERICGGTADAADGFTATLMMLAATAIPANLLEAMWALLGPRDEDLGENWQLGEGEVEGQWSGGAASAAAAAMLRFMLRRAAAVDNYTVATSGGTAGLALDSLSAKLLGPAGGSIMFLLKEDAVNLRRLLAFDVALLSILFGAKVVIAYETSALSRESGLLLCSSVFDARRGNRFADILCADHRACASDSAAAAADALKKIALADPNRIENACLLQQVEELASALHSKPLSYAQPFLFLANTSNQITQVLIPAKARPSEFFVTLRRDAAYEEVPLRRADRQAFPDEIDAKDIEGGDLFFSATVGGEVPVLDNPAHIAPAPPEYDRGEGNLFPFAGSRRSAPSEEEALSKAAGSKRNQIDAHGQNISQPARAGNTKIERASGKNRKTENNVTEHQTAARGRAAAPPTETKTTEKRQKCPPRESPLSRDERAPHDGLSAGAAEPRPPRGDSDDDRHKHETPHGVSDKAAEPPAVPSAEPRGPSTELIGGNWKSLPKTKPRRTSSGLRRKHQASASVHKHRTHGSSDDDSEDGEATYGFGSCRGRQRRSTLGGKKRSGTDRTAEFLKKATCVDKLEKFSRSGESPKAQNGTADVACDRLGERGNELSPPRAPASSPPPPGKQADHGIDQREIVPPNAQYGITTVVDPRQVRLPSSDDGDPAEEEDARDVEEGEEDVAGQWDSNYDVCLPTYDTDHAAQEEKDFDLASNNGTGGALPAADHAISAINDWVIADTDASAGVGTDWSEEDEDAPAADDGRSTNVEVATHGYTSDDSAADDESKRARATRDSSPPQHYPASPLAPSTPSSLPTPADTDNDTAILELDRNSGGDTDSNDDHAPPTDTGDAPPLCSEGELTPSTDEECAVVQDDARKKQENSSHERKDDGVRWEIDLDSDQGDYSDASDDCKIPDGPRVAPEKDIKNKQLEKSESDSCGGQGDPSTEPQQPLWEVYSPYDNSDSDDKAGNRKDPKLDGAALDMRSSRLRADAKSITSYVNDINEAVRDGGSAAAEFFARSEQSCIDSEDDARHLDKSRATLASDLDDHQSDQPRESLAPLDPETRSKMYTSLAVTCRLILRGMRHAQDAASAGVAELLTETNRIKMMLN

SEQ ID NO: 51 - protein, 2241 aa, Human herpesvirus 5
MKVTQASCHQGDIARFGARAGNQCVCNGIMFLHALHLGGTSAVLQTEALDAIMEEGARLDARLERELQKKLPAGGRLPVYRLGDEVPRRLESRFGRTVHALSRPFNGTTETCDLDGYMCPGIFDFLRYAHAKPRPTYVLVTVNSLARAVVFTEDHMLVFDPHSSAECHNAAVYHCEGLHQVLMVLTGFGVQLSPAFYYEALFLYMLDVATVPEAEIAARLVSTYRDRDIDLTGVVRESADTAATTTTAAPSLPPLPDPIVDPGCPPGVAPSIPVYDPSSSPKKTPEKRRKDLSGSKHGGKKKPPSTTSKTLATASSSPSAIAAASSSSAVPPSYSCGEGALPALGRYQQLVDEVEQELKALTLPPLPANTSAWTLHAAGTESGANAATATAPSFDEAFLTDRLQQLIIHAVNQRSCLRRPCGPQSAAQQAVRAYLGLSKKLDAFLLNWLHHGLDLQRMHDYLSHKTTKGTYSTLDRALLEKMQVVFDPYGRQHGPALIAWVEEMLRYVESKPTNELSQRLQRFVTKRPMPVSDSFVCLRPVDFQRLTQVIEQRRRVLQRQREEYHGVYEHLAGLITSIDIHDLDASDLNRREILKALQPLDDNAKQELFRLGNAKMLELQMDLDRLSTQLLTRVHNHILNGFLPVEDLKQMERVVEQVLRLFYDLRDLKLCDGSYEEGFVVIREQLSYLMTGTVRDNVPLLQEILQLRHAYQQATQQNEGRLTQIHDLLHVIETLVRDPGSRGSALTLALVQEQLAQLEALGGLQLPEVQQRLQNAQLALSRLYEEEEETQRFLDGLSYDDPPNEQTIKRHPQLREMLRRDEQTRLRLINAVLSMFHTLVMRLARDESPRPTFFDAVSLLLQQLPPDSHEREDLRAANATYAQMVKKLEQIEKAGTGASEKRFQALRELVYFFRNHEYFFQHMVGRLGVGPQVTELYERYQHEMEEQHLERLEREWQEEAGKLTVTSVEDVQRVLARAPSHRVMHQMQQTLTTKMQDFLDKEKRKQEEQQRQLLDGYQKKVQQDLQRVVDAVKGEMLSTIPHQPLEATLELLLGLDQRAQPLLDKFNQDLLSALQQLSKKLDGRINECLHGVLTGDVERRCHPHREAAMQTQASLNHLDQILGPQLLIHETQQALQHAVHQAQFIEKCQQGDPTTAITGSEFEGDFARYRSSQQKMEEQLQETRQQMTETSERLDRSLRQDPGSSSVTRVPEKPFKGQELAGRITPPPADFQQPVFKTLLDQQADAARKALSDEADLLNQKVQTQLRQRDEQLSTAQNLWTDLVTRHKMSGGLDVTTPDAKALMEKPLETLRELLGKATQQLPYLSAERTVRWMLAFLEEALAQITADPTHPHHGSRTHYRNLQQQAVESAVTLAHQIEQNAACENFIAQHQEATANGASTPRVDMVQAVEAVWQRLEPGRVAGGAARHQKVQELLQRLGQTLGDLELQETLATEYFALLHGIQTFSYGLDFRSQLEKIRDLRTRFAELAKRRGTRLSNEGVLPNPRKPQATTSLGAFTRGLNALERHVQLGHQYLLNKLNGSSLVYRLEDIPSVLPATHETDPALIMRDRLRRLCFARHHDTFLEVVDVFGMRQIVTQAGEPIHLVTDYGNVAFKYLALRDDGRPLAWRRRCSGGGLKNVVTTRYKAITVAVAVCQTLRTFWPQISQYDLRPYLTQHQSHTHPAETHTLHNLKLFCYLVSTAWHQRIDTQQELTAADRVGSGEGGDVGEQRPGRGTVLRLSLQEFCVLIAALYPEYIYTVLKYPVQMSLPSLTAHLHQDVIHAVVNNTHKMPPDHLPEQVKAFCITPTQWPAMQLNKLFWENKLVQQLCQVGPQKSTPPLGKLWLYAMATLVFPQDMLQCLWLELKPQYAETYASVSELVQTLFQIFTQQCEMVTEGYTQPQLPTGEPVLQMIRVPRQDTTTTDTNTTTEPGLLDVFIQTETALDYALGSWLFGIPVCLGVHVADLLKGQRILVARHLEYTSRDRDFLRIQRSRDLNLSQLLQDTWTETPLEHCWLQAQIRRLRDYLRFPTRLEFIPLVIYNAQDHTVVRVLRPPSTFEQDHSRLVLDEAFPTFPLYDQDDNSSADNIAASGAAPTPPVPFNRVPVNIQFLRENPPPIARVQQPPRRHRHRAAAAADDDGQIDHVQDDTSRTADSALVSTAFGGSVFQENRLGETPLCRDELVAVAPGAASTSFASPPITVLTQNVLSALEILRLVRLDLRQLAQSVQDTIQHMRFLYLL

SEQ ID NO: 52 - protein, 3149 aa, Human herpesvirus 4
MSNGDWGQSQRTRGTGPVRGIRTMDVNAPGGGSGGSALRILGTASCNQAHCKFGRFAGIQCVSNCVLYLVKSFLAGRPLTSRPELDEVLDEGARLDALMRQSGILKGHEMAQLTDVPSSVVLRGGGRVHIYRSAEIFGLVLFPAQIANSAVVQSLAEVLHGSYNGVAQFILYICDIYAGAIIIETDGSFYLFDPHCQKDAAPGTPAHVRVSTYAHDILQYVGAPGAQYTCVHLYFLPEAFETEDPRIFMLEHYGVYDFYEANGSGFDLVGPELVSSDGEAAGTPGADSSPPVMLPFERRIIPYNLRPLPSRSFTSDSFPAARYSPAKTNSPPSSPASAAPASAAPASAAPASAAPASAAPASAAPASAAPASAAPASSPPLFIPIPGLGHTPGVPAPSTPPRASSGAAPQTPKRKKGLGKDSPHKKPTSGRRLPLSSTTDTEDDQLPRTHVPPHRPPSAARLPPPVIPIPHQSPPASPTPHPAPVSTIAPSVTPSPRLPLQIPIPLPQAAPSNPKIPLTTPSPSPTAAAAPTTTTLSPPPTQQQPPQSAAPAPSPLLPQQQPTPSAAPAPSPLLPQQQPPPSAARAPSPLPPQQQPLPSATPAPPPAQQLPPSATTLEPEKNHPPAADRAGTEISPSPPFGQQPSFGDDASGGSGLVRYLSDLEEPFLSMSDSEEAESDLASDIPTTEDEDMFEDEVFSNSLESGSSAPTSPITLDTARSQYYQTTFDIETPEMDFVPLESNIARIAGHTYQEQAIVYDPASNREVPEADALSMIDYLLVTVVLEQGLIRSRDRSSVLNLLEFLKDWSGHLQVPTLDLEQLLTSELNIQNLANMLSENKGRAGEFHKHLAAKLEACLPSLATKDAVRVDAGAKMLAEIPQLAESDDGKFDLEAARRRLTDLLSGGDQEAGEGGGEPEDNSIYRGPHVDVPLVLDDESWKRLLSLAEAARTAVARQQAGVDEEDVRFLALLTAIEYGAPPAASVPPFVHNVAVRSKNAALHVRRCTADIRDKVASAASDYLSYLEDPSLPTVMDFDDLLTHLRHTCQIIASLPLLNIRYTSIEWDYRELLYLGTALSDMSGIPWPLERVEEDDPSIAPLPEFETVAKKQKELETTRENEKRLRTILDDIEAMLGLAGVASAPGAPISPASPSATPANHDNPEATPPLADTAALTIPVIEKYIANAGSIVGAAKNPTYIRLRDTIQQIVRSKKYLMNILKSITFYTIDNYIASFEESIDHLYRDLPVLDPEVQDGIDRILDPMVSEALHTFEMGNRLTLEPARLVALQNFATHSTLKETAAAVNLLPGLLAVYDATITGQAPEDALRLLSGLQNQLSQTLIPGKLKKRFLSYLQKLKNNNNDQLRQKEVQAWRLEAEGFKPATEEQLEAFLDTAPNKELKRQYEKKLRQLMETGRKEKEKLREQEDKERQERRAREANEAWARIRKALGARPEPAPTSPDDWNTLLASLLPDNTDSAAAAAAAVARNTDILDSLTQILAAMLLGITRVRRERLRSLLVDDGGAAERMEAAEPGWFTDIETGPLARLDAWPATPAATAKEGGGGRGAEEAAGALFRARTAADAIRSALAQTRQALQSPDMKSAVVNTDLEAPYAEYERGLAGLLEKRRAAEAALTAIVSEYVDRTLPEATNDPGQANLPPPPTIPQATAPPRLASDSALWPKKPQLLTRRERDDLLQATGDFFSELLTEAEAAEVRALEEQVRESQTLMAKAHEMAASTRRGFHTALEAVLSRSRDEAPDDELRSLLPSPPKAPVQAPLEAALARAAAGNGSWPYRKSLAAAKWIRGICEAVRGLSEGALALAGGAGAWLNLAAAADGEIHELTRLLEVEGMAQNSMDGMEELRLALATLDPKRVAGGKETVADWKRRLSRLEAIIQEAQEESQLQGTLQDLVTQARGHTDPRQLKIVVEAARGLALGASAGSQYALLKDKLLRYASAKQSFLAFYETAQPTVFVKHPLTNNLPLLITISAPPTGWGNGAPTRRAQFLAAAGPAKYAGTLWLETESPCDPLNPAYVSADTQEPLNYIPVYHNFLEYVMPTVLENPEAFSLTPAGRPQAIGPPQDDQERRRRTLASVASARLSAAAADSYWDTWPDVESNAGELLREYVSAPKALMEDLADNPIVAMTLLAHASLIASRNHPPYPAPATDREVILLEQREMMALLVGTHPAYAAAFLGAPSFYAGLGLVSALARDGGLGDLLSDSVLTYRLVRSPASGRGGMPSTTRGSNDGEDARRLTRHRIAGPPTGFIFFQDAWEEMDTRAALWPHPEFLGLVHNQSTARARACMLLLARRCFAPEALQQLWHSLRPLEGPVAFQDYLRDFVKQAYTRGEELPRAEGLEVPRETPSSYGTVTGRALRNLMPYGTPITGPKRGSGDTIPVSVFEAAVAAAFLGRPLTLFVSSQYLFNLKTLGQVRVVAPLLYCDGHSEPFRSLVETISLNFLQDLDGYSESFEPEMSIFARQAVWLRELLTEARAAKPKEARPPTVAILANRKNIIWKCFTYRHNLPDVQFYFNAAGASRWPTDVLNPSFYEHEDPPLPVGYQLPPNPRNVQELFSGFPPRVGHGLVSGDGFQSADNTPASSDRLQQLGGGETDQGEKGSTTAESEASGPPSPQSPLLEKVAPGRPRDWLSPTSSPRDVTVTPGLAAPITLPGPRLMARPYFGAETRASESPDRSPGSSPRPWPKDSLELLPQPAPQQPPSSPWASEQGPIVYTLSPHSTPSTASGSQKKHTIQIPGLVPSQKPSYPPSAPYKPGQSTGGIAPTPSAASLTTFGLQPQDTQASSQDPPYGHSIMQREKKQQGGREEAAEIRPSATRLPTAVGLRPRAPVVAAGAAASATPAFDPGEAPSGFPIPQAPALGSGLAAPAHTPVGALAPRPQKTQAQRPQDAAALPTPTIKAVGARPVPKATGALAAGARPRGQPTAAPPSAASPPRVSLPVRSRQQQSPAIPLPPMHSGSEPGARPEVRLSQYRHAGPQTYTVRKEAPPSAASQLPKMPKCKDSMYYPPSGSARYPAPFQALSFSQSVASPAPSSDQTTLLWNTPSVVTQFLSIEDIIREVVTGGSTSGDLVVPSGSPSSLSTAAPEQDLRYSLTLSQASRVLSRFVSQLRRKLERSTHRLIADLERLKFLYL

SEQ ID NO: 53 - protein, 2149 aa, Murid herpesvirus
MKIVRASRDQSAPVYGPRAGSQCMSNCFTFLHTCYLMGIDPVLDTTSLDAVLDSGARLDAIADEKVKRQALTDHPYRLGTEIPTVIETPAGITGHALSRPFNGTAETQDLGGYKCLGILDFLTYARGKPLPVYIIVTVGVHTRGVIVARGATYVFDPHTTDLSAEAAVYVCDDFTEAISALSFFGAMIGDFYYDAVLVYFTRCRTTLISPSELLVQIMDQYKDPDIDASVMSGSGGGGGGPSISSSAASASASVSPLPSGAASDGVTNGAGAQTSSKSGKKRRAPDVPPQQKGTAKTLRRSRRPIRLPERLSEVIVEDVQTIEHFRTATQSLPVKPPQEWTMYCGEEPFYRQYFVDVGRQLLAHAIDTYVSLDKTTDESAVQRFESLMGLSDELDAVIAAARATELSAPPLYKTYLSQRVSASAVTRTDRLLASKILALFEEGSATDFETVSSWLRELLQQLPVENTTTTEAKIAAFVAQHPIPGERAFVCLRNSQVKSLGELLSVKRETLKVKYMENDATYRKILDAISKLGLASNAAAAIQSADTSHLDSEQLASLAQAAEAYAQSAYESCQAKMTELLSTNHNRILTGSLPDSDIAAVLAAFKAVSENVRALREVELLKSQVHVQLAQLTEDLLYLQTAEVKLEVTPSAEIKKLRAEYETARKQISDEEMRIKELIENMEDMITDSSSSPPPPEMLDMLRTQIEEVESMTVDEQDARRADKVLGKLSTLDEAEAKATEFAQSLSTVNIPSLNEIKGVKMLKSVLETNADLRRAYVQAVTGMLENALKQLAEGNLPSDDMMSRITALAEQLPTGRQVRADLLDSTDIVSQMSRRLRYAANQKNSTQSLEDVLNFFSENDDMIRKLLKTSWGKPVATVYRRVQVEYDRKMEELRESEWLKRVKETDIDSPQTLERLLKTAPNETILAKHAPDMHARLKKRMQSEAEKRTADMKRLYEEMRKKVDTDLKVVSDSFSSQTPSVFSSIDLKSCGTSLVRLTKEDRKAAIATFNANLTKSLNSLLASLVTVETATIAAILKGADPEATSSTGSGGGGADRQKHTSTLENNISTLLGWQQKVLLPETERDLLTIAHLLTALTHMRKNWRNPAAAFSSTPHTAAYRNFAELANEIESRRTETLARYKSKYAELNASIHDNTKANDVTIKPEDTFTAAFRDTVKAMAAPFSTELQARLQARENELKDELSDLNVKLKTKLERYLAKRSSQDARWRDLITQHRIRLPEGLDVDTNRLRTDTVVTLNGIIRSAATTLPYITAKRGLEWTTEFILAAIEEKKDADPGMFAQLATLLDNSQAIARKIEEKIVYNTKVEAEMLETAPEHTRESLSNLQLMLGQLEAKRVVGGEARYKILSDAILTKQNKLAFAEDLEKLSARYFELARDIRSSKYGLDFDAQLLKTSLLKSEIGQHKKDPPSTGEEVGLPEESTTAAKISISSLLLGIAALEKYIVAHRTLLDNFVSSQPLISQAENIPALVGGPGEDDKPGQEPFDWTRIDLSRLSACDVSTALYRGTDVFGERRVMTARGIQLYLYATHGNFVFEAFSHVRGTKGLLSSSSGGGNGSGASSRQKDQQNATVTRRYRSVTVLASIAATLQTFWSEISRYDIRELLVESGRDPESAADQRDRRLNTVMNLKLFVYVMTVAWSEATPPVEPGSPEATHALEVSLLDFSTLMAALHPEYVYAITTQPVDATLRGLIARLDRRTVDAAMNTQENPPPYDMRELKAFCLDTKQWTQEDIRPQMWMSDLVKQICTSHPRNRDASTSTKLFLYMLATKVLPRDILRCLWVQFRPAYASSIASLEELVSALCDSFFRIYGTTSETVSARLKTGEKVERQVVLRHKPTMSLLDEFSQQEAVLDYILGSYVFAIPMTVGIHVTNIVNGRYRLIVRHLENLPSDPDFVKVVRSRDLSFDRFGWSYTVQNPVERSWFSLQEDRLRHLLTNPPQQDRTPLVVYDSNTNYAVNAMMPPLKAPPAASRVHLTVKNPFSTMRMIPHEDEGDDAATSETPFTSLPINIDFLRRDPPRLKRASGDSSSSVPAEDRDPDARPQDSVPDQSLSEPLFSQTKVSSIVPKDAVTTLSNESISQTFQIHPFRALSSAIMAAIEILQETRLQLDTFESDMCEAIRRIKILYLH

SEQ ID NO: 54 - protein, 2635 aa, Human herpesvirus 8
MAAQPLYMEGMASTHQANCIFGEHAGSQCLSNCVMYLASSYYNSETPLVDRASLDDVLEQGMRLDLLLRKSGMLGFRQYAQLHHIPGFLRTDDWATKIFQSPEFYGLIGQDAAIREPFIESLRSVLSRNYAGTVQYLIIICQSKAGAIVVKDKTYYMFDPHCIPNIPNSPAHVIKTNDVGVLLPYIATHDTEYTGCFLYFIPHDYISPEHYIANHYRTIVFEELHGPRMDISRGVESCSITEITSPSVSPAPSEAPLRRDSTQSQDETRPRRPRVVIPPYDPTDRPRPPHQDRPPEQAAGYGGNKGRGGNKGRGGKTGRGGNEGRGGHQPPDEHQPPHITAEHMDQSDGQGADGDMDSTPANGETSVTETPGPEPNPPARPDREPPPTPPATPGATALLSDLTATRGQKRKFSSLKESYPIDSPPSDDDDVSQPSQQTAPDTEDIWIDDPLTPLYPLTDTPSFDITADVTPDNTHPEKAADGDFTNKTTSTDADRYASASQESLGTLVSPYDFTNLDTLLAELGRLGTAQPIPVIVDRLTSRPFREASALQAMDRILTHVVLEYGLVSGYSTAAPSKCTHVLQFFILWGEKLGIPTEDAKTLLESALEIPAMCEIVQQGRLKEPTFSRHIISKLNPCLESLHATSRQDFKSLIQAFNAEGIRIASRERETSMAELIETITARLKPNFNIVCARQDAQTIQDGVGLLRAEVNKRNAQIAQEAAYFENIITALSTFQPPPQSQQTFEVLPDLKLRTLVEHLTLVEAQVTTQTVESLQAYLQSAATAEHHLTNVPNVHSILSNISNTLKVIDYVIPKFIINTDTLAPYKQQFSYLGGELASMFSLDWPHAPAEAVEPLPVLTSLRGKIAEALTRQENKNAVDQILTDAEGLLKNITDPNGAHFHAQAVSIPVLENYVHNAGVLLKGEKSERFSRLKTAIQNLVSSESFITVTLHSTNLGNLVTNVPKLGEAFTGGPHLLTSPSVRQSLSTLCTTLLRDALDALEKKDPALLGEGTTLALETLLGYGSVQDYKETVQIISSLVGIQKLVRDQGADKWATAVTRLTDLKSTLATTAIETATKRKLYRLIQRDLKEAQKHETNRAMEEWKQKVLALDNASPERVATLLQQAPTAKAREFAEKHFKILLPVPADAPVQASPTPMEYSASPLPDPKDIDRATSIHGEQAWKKIQQAFKDFNFAVLRPADWDALAAEYQRRGSPLPAAVGPALSGFLETILGTLNDIYMDKLRSFLPDAQPFQAPPFDWLTPYQDQVSFFLRTIGLPLVRALADKISVQALRLSHALQSGDLQQATVGTPLELPATEYARIASNMKSVFNDHGLQVRSEVADYVEAQRADAHTPHVPRPKIQAPKTLIPHPDAIVADGLPAFLKTSLLQQEAKLLALQRADFESLESDMRAAEAQRKASREETQRKMAHAITQLLQQAPSAISGRPLSLQDPVGFLEGIIYDKVLERESYETGLEGLSWLEQTIKSITVYAPVEEKQRMHVLLDEVKKQRANTETALELEAAATHGDDARLLQRAVDELSPLRVKGGKAAVESWRQKIQTLKSLVQEAEQAGLLLATIDTVAGQAQETISPSTLQGLYQQGQEAMAAIKRFRDSPQLAGLQEKLAELQQYVKYKKQYLEHFEATQSVVFTAFPLTQEVTIPALHYAGPFDNLERLSRYLHIGQTQPAPGQWLLTLPTFDPTRPACVPAGGHEPPLHRQVVFSSFLEAQIRLALSVAGPVPGRGLPGTPQIRRGVEAAACFLHQWDEISRLLPEVLDTFFHNAPLPAESSSNAFLAMCVLTHLVYLAGRAVLGPREPEHAAPDAYPREVALAPRDLTYLLLAMWPSWISAILKQPSHAEAAHACLVTLPTMLKAVPYLTLEASAGPLPADMRHFATPEARLFFPARWHHVNVQEKLWLRNDFMSLCHRSPGRARIAVLVWAVTCLDPEVIRQLWSTLRPLTADESDTASGLLRVLVEMEFGPPPKTPRREAVAPGATLPPYPYGLATGERLVGQAQERSGGAGKMPVSGFEIVLGALLFRTPLRIFSTASTHRISDFEGGFQILTPLLDCCPDREPFASLAAAPRRTVPLGDPCANIHTPEEIQIFARQAAWLQYTFANYQIPSTDNPIPIVVLNANNNLENSYIPRDRKADPLRPFYVVPLKPQGRWPEIMTTATTPCRLPTSPEEAGSQFARLLQSQVSATWSDIFSRVPERLAPNAPQKSSQTMSEIHEVAATPPLTITPNKPTGTPHVSPEADPITERKRGQQPKIVADNMPSRILPSLPTPKPREPRITLPHALPVISPPAHRPSPIPHLPAPQVTEPKGVLQSKRGTLVLRPAAVIDPRKPVSAPITRYERTALQPPRTEGEGRRPPDTQPVTLTFRLPPTAPTPATAALETKTTPPSTPPHAIDISPPQTPPMSTSPHARDTSPPAEKRAAPVIRVMAPTQPSGEARVKRVEIEQGLSTRNEAPPLERSNHAVPAVTPRRTVAREIRIPPEIKAGWDTAPDIPLPHSSPESSPPTSPQPIRVDDKSPLPNLVERYARGFLDTPSVEVMSLENQDIAVDPGLLTRRIPSVVPMPHPIMWSPIVPISLQNTDIDTAKITLISFIRRIKQKVAALSASLAETVDRIKKWYL

SEQ ID NO: 55 - protein, 2077 aa, Human herpesvirus 6 U31
MKIITSSTNQNDSKYGPRAGKQCMSNSFSFLHTVYLNGINNSLNAGTIDAIMEEGYHLDTAGTLALMLNNSDSQDYRLPTEIPKRIHSRYGVTQHELSRPFNGTLDTQKIDNEVYLGLIDFILYGKSKNGPTFAVITIGVLSRALFFLNNTLYLFDSHPTEREATAAIYICQNIEEVYELLTTHGTEGFYYDASLIFFIETSNLSLSSHDAELLILKTYKDPDIAIALDKFSSTEIHEIKKTDDIGSQQDLVADKTTDLEHAPHKRKKNSHSLELELNDKKKKDTASLTYYATEVDLIPSFYELRSQFQSLFHDLKSFPIMKSNFNWTIYLQDSPINPNQPFATPFLWNRVFHLLCQIIDVFVGVGSTNDDSSKQQQQTIFINYLLPFKDFSEVFNEALAACQENNLDIIFIYNNYLCKTTTFRTLERILLSKFLAIVDNDHKKHYEWVKSWTTQMFQGMPKKLDDIENYLKAYVDHNPVKHFHEFICLNKAEKYKVAVLLNEKRKEIQEAIEREKNSFAQLSNFIDKLGETPALPIESENVHKVHTSDITEGIVPRFITESIELPNISTLNNTQQISLDKQLNEKLTNTIHTLTNKFTKIVQDNYNNIAAGFMPVTELNCLFAYLVNLYFNIEVLKHSGLNINTVLLQEVEKLYDNTQFLRFGTSHFNINNLSNFTLSIRKMFVDFYNSQKPSDRASEILAAIESILADPSKNKTVVNIEMIKSQLEELGKMEISTTENKQTAAITKQILGDQELTPIYDFLHHLSAYNLPNTTTVKNLHLHFILEQRPDIAMTLHDKIQSILDIYVDDMLNDITVPEQTFSTVLFLVDLFPNSTEKTALFESVLTLRQLAKKCANLKTLDEFDDLAQFITTNSEQLQNMMRQHFGKKIPTLMGHIKFLYSQKIITTEEKNWIQRAKTVVITSPEELTAFLATAPTKHALQTCKPDLDKALQRHMEEQMKQTAENDKKHILTIRSTLEKRLTDILLILKDGQFSSLETMHLNLLETFLKQLQDNNVIIHFTHALLPVLKDIETTISKIISDVIEKILIKTPLNPEQMSKEEQKYTPLLSFLSKFKKTTFCTEDVKTEIEQVQKSITFLKKIATSTNKHTRLSHSIYGQELNLYEERITELRKETNKMKEQLSKEYALAEKKILLSSQDAKTDKIYLVLNTHTLKEIKNTQFKETAFAKALTVEVNNKENQLQELLNHFNAHLKAKMDQNHITKLSFDTKWTAFVSDSRLYIPDFINIKLQDFISDPFKVISQLMNKATNEMPYIQAEITLKWLTKLIHDINKFCLSAISEFGKEAIPFNYAALRDLEYQINTKYVEIENKVICNETVENTKNIPKLTKLLKQLDPKRVAGGQEQYQTLMNKILTSETSMQQTYEKEQLKKEYFEIVNNVASFKLAFNFPQQLQNVERLIEKFKSLPKSQPFEKFPQENDLLSDSLNTENYINGLRALLNFITAAQNYIQNTLLKQWAVFQQQNFIPIDYSVANVKPISDLYARLRIERERQVFYQVNSVFGTHLIVDDTGVPLQFHNIFNNAIVKFFSLNYKQINVPEDTPRLVSSQYKLLSVCKSFIMILQQFWENIITLDLGPYLRDGTQNFKRELIPIVNLKLFIYCITQAWTASEDSTVSTAFELPIKQFTLLILCSHPEYLYGCLSHSTDLVINSLAKSIDKNSLYNTFVVSHNPPEKPMHLMRNICIDTQLWQPAKLMKDTFQQTFFTQLCPKNEKFFIYLTAFLILPYKFMNYIWIQYKPAIFTQRSYQNLIKDLCSEYVHQNKITTSSVTPHEPDTIKSGERITSKITVHKAQNTPTLTRLQAQEYVFDYILYSFLTGYEMTFAMYIDIIEKTYLLCMRHLENVLHDKDFQSVLRARTFDIDYILKQSWTKNIVEHSLFSVQLDKIVSYLNHTNRATPNIPLILFNYDNEVVNVYLPPMSTNPKKVAFYIKNPFHFPVQEYEATNLISFHLYPKTTDILNQLPPNNTESTRPGKQTSETLTNKNLSEPKFKKPAVTGLMPKSQSIILSTDTNVPETSPDVKANTASAAIKDVTLAREKINEFSESINTTISKLKSMYL

SEQ ID NO: 56 - protein, 308 aa, Tortoise herpesvirus
MADGRPALSGEWGELLAYSRVNQGDPVFGSNSGVQCVINSVVFLRSIYLYGLKSVLSREAITTILHQGSAIFTAVSTDGRYLLCDEIPLFISTGGITTIGAISKSYGNMPIIGDSGALGVEGLVMANGYEFVEIIKSRSAHPLDYIVAIIGKSAIALYRDNDFVFIFEPHGYKDSGAFVSRIYLNQLYSFLLSYGSESQYNAVFVYFALTKTDNPEAYPLHMLRNYITEKYGAVDAVPELQRNIQVRIALQLQQPKTKRTLDEYIQRAVRLQQGKRKTNLPLLVSNEALDDITRSSSRNRAKSSRREA